(19) United States
(12) Patent Application Publication
(10) Pub. No.: US 2026/0124192 A1
(43) Pub. Date: May 7, 2026

(54) COMPOUNDS, COMPOSITIONS, AND METHODS OF TREATING RNA VIRAL INFECTIONS

(71) Applicant: Model Medicines, Inc., San Diego, CA (US)

(72) Inventors: Daniel Haders, San Diego, CA (US); Tyler Umansky, San Diego, CA (US); David Garvey, San Diego, CA (US); Virgil Woods, San Diego, CA (US); George Nicola, San Diego, CA (US)

(21) Appl. No.: 19/434,767

(22) Filed: Dec. 29, 2025

Related U.S. Application Data
(63) Continuation of application No. PCT/US2025/021929, filed on Mar. 28, 2025.
(60) Provisional application No. 63/571,351, filed on Mar. 28, 2024.
(60) Provisional application No. 63/667,563, filed on Jul. 3, 2024.
(60) Provisional application No. 63/682,664, filed on Aug. 13, 2024.
(60) Provisional application No. 63/743,555, filed on Jan. 9, 2025.
(60) Provisional application No. 63/745,727, filed on Jan. 15, 2025.

(51) Int. Cl. A61K 31/454; A61P 31/14

(52) U.S. Cl. CPC A61K 31/454; A61P 31/14

(57) ABSTRACT

Disclosed herein are broad-spectrum antiviral compounds that can prevent or treat disorders related to RNA viral infections. Also disclosed are methods and composition for treating RNA viral infections, including symptoms of the RNA viral infections. The methods, for example, can include administering pharmaceutical compositions comprising one or more of the compounds disclosed herein to a patient in need.

### RELATED APPLICATIONS

The present application is a continuation application of PCT Application No. PCT/US2025/021929, filed on Mar. 28, 2025, which claims priority to U.S. Provisional Application No. 63/571,351, filed on Mar. 28, 2024; U.S. Provisional Application No. 63/667,563, filed on Jul. 3, 2024; U.S. Provisional Application No. 63/682,664, filed on Aug. 13, 2024; U.S. Provisional Application No. 63/743,555, filed on Jan. 9, 2025; and U.S. Provisional Application No. 63/745,727, filed on Jan. 15, 2025. The entire content of these related applications is hereby expressly incorporated by reference in its entirety.

### BACKGROUND

### Field

The present disclosure generally relates to the use of drugs for the treatment of RNA viral infections. More specifically, the disclosure describes methods for the treatment of an RNA viral infection and/or treatment or prevention of symptoms of an RNA viral infection by administering pharmaceutical compositions.

### Description of the Related Art

From the Justinian plague in the premodern times (500s A.D.), to the COVID-19 outbreak in 2019, the history of humankind can be charted along the timeline of pandemic outbreaks. Zoonotic viruses, particularly RNA viruses, or viruses with an RNA part of their lifecycle, remain the primary pandemic catalyzers of the modern era (e.g., Ebola virus; human immunodeficiency virus; multiple influenza A virus subtypes, and Coronaviruses). In the late 1890s, an undefined influenza A virus subtype triggered the first pandemic of the modern era (the so-called Russian pandemic). Multiple influenza A virus subtypes followed suit afterward, triggering several pandemics throughout the 1900s and early 2000s. The Great flu in 1918 (influenza A/H1N1 virus), brought worldwide death in epic proportions, only matched by the COVID-19 pandemic in 2019. Just shy of 25 years into the 21^{st} century, Coronaviruses have made a mark with three widespread outbreaks: the severe accurate respiratory syndrome (SARS) (2002-2004), Middle Eastern respiratory syndrome (MERS) (2012) and COVID-19 (2019-2023), mentioned above. In the aftermath of the COVID-19 pandemic, epidemiological surveillance programs have shown that zoonotic virus emergence is far from winding down.

When a viral pathogen is known, vaccines have been important in keeping outbreaks at bay, like contemporary influenza, in many, but not all, cases and more recently, managing the COVID-19 pandemic. Vaccine development is, however, a lengthy and sometimes an impossible task, usually initiated only after proof of a virus's pandemic potential. Plus, vaccine storage and distribution can be an obstacle. Small-molecule antiviral drugs, on the other hand, can offer a quicker therapeutic approach, but often have limited activity spectrum. Before COVID-19 vaccines were developed, available small-molecule antivirals did little to abate the COVID-19 pandemic's heavy death toll. This grim experience underlined the need to develop safe and effective, stockpiled, broad-spectrum antiviral drugs that can be implemented at the very onset of a viral outbreak. There is an urgent need for compositions and methods for preventing, delaying the onset of, or treating an inflammatory effect of an infection or a disease caused by a RNA virus.

### SUMMARY

Disclosed herein includes broad-spectrum antiviral compounds, and the use of the compounds and pharmaceutical compositions thereof for the treatment and prevention of disorders related to RNA viral infections.

Disclosed herein includes a compound of Formula I:

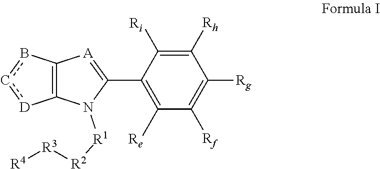

wherein A is independently:
   1) Nitrogen or
   2) —C(R_{X});
[symbol] D is:
   1) C(Rₐ)═C(R_{b})—C(R_{c})═C(R_{d}) or
   2) S—C(R_{c})═C(R_{d});
R¹ is:
   1) Lower alkyl;
   2) Haloalkyl;
   3) Cycloalkylalkyl; or
   4) Carbonyl;
R² is:
   1) Phenyl;
   2) Cycloalkyl; or
   3) Absent;
R³ is:
   1) Lower alkyl;
   2) Alkoxy; or
   3) Absent;
R⁴ is:
   1) Cyclalkyl;
   2) Cycloamine; or
   3) Absent;
R_{X} is:
   1) Hydrogen
   2) Deuterium
   3) Halogen
   4) Lower alkyl
   5) Haloalkyl;
   6) Cycloalkyl;
   7) Cycloalkylalkyl; or
   8) R¹—R²—R³—R⁴;
Rₐ, R_{b}, R_{c}, and R_{d} are independently selected from:
   1) Hydrogen;
   2) Deuterium;
   3) Alkoxy;
   4) Halogen;
   5) Hydroxy
   6) Nitro; and
   7) Amino; and
Rₑ, R_{f}, R_{g}, Rₕ and Rᵢ are independently selected from:
   1) Hydrogen;
   2) Deuterium
   3) Alkoxy;
   4) Halogen
   5) Nitro;
   6) Carboxyl; and
   7) Carboxylic ester.

In some embodiments, A is CR_{X} or nitrogen; [symbol] is S—C(R_{c})═C(R_{d}) or C(Rₐ)═C(R_{b})—C(R_{c})═C(R_{d}); R_{X} is lower alkyl, haloalkyl or cycloalkylalkyl; R¹ is lower alkyl or carbonyl; R² or phenyl or absent; R³ is lower alkyl alkoxy or absent; R⁴ is cycloalkyl, cycloamino or absent; Rₐ, R_{b}, R_{c} and R_{d} are independently selected from hydrogen, halogen, hydroxyl, alkoxy, nitro or amino; and/or Rₑ, R_{f}, R_{g}, Rₕ and Rᵢ are independently selected from hydrogen, halogen, hydroxyl, alkoxy or carboxyl.

In some embodiments, A C(R_{X}); R_{X} is hydrogen or lower alkyl, [symbol] is S—C(R_{c})═C(R_{d}), R¹ is lower alkyl, R² is phenyl, R³ is lower alkyl or alkoxy, R⁴ is cycloalkyl or cycloamino, R_{c} and R_{d} are hydrogen or deuterium, and/or Rₑ, R_{f}, R_{g}, Rₕ and Rᵢ are independently selected from hydrogen, halogen, alkoxy or carboxyl. In some embodiments, R_{X} is hydrogen; Rᵢ is lower alkyl; R² is phenyl, R³ is alkoxy, R⁴ is cycloalkyl or cycloamino, R_{c} and R_{d} are hydrogen, Rₑ, R_{f}, Rₕ and Rᵢ are independently selected from hydrogen or deuterium, and/or R_{g} is hydrogen, deuterium or halogen. In some embodiments, R_{X} is hydrogen; R¹ is methyl, R² is phenyl, R³ is ethoxy, R⁴ is cycloamino, R_{c} and R_{d} are hydrogen, Rₑ, R_{f}, Rₕ and Rᵢ are independently selected from hydrogen or deuterium, and/or R_{g} is halogen.

In some embodiments, A is nitrogen, [symbol] is C(Rₐ)═C(R_{b})—C(R_{c})═C(R_{d}), R¹ is lower alkyl or carbonyl, R² absent or phenyl, R³ is lower alkyl or alkoxy, R⁴ is cycloalkyl or cycloamino, Rₐ, R_{b}, R_{c} and R_{d} are independently selected from hydrogen, halogen, hydroxyl, alkoxy, nitro or amino, and/or Rₑ, R_{f}, R_{g}, Rₕ and Rᵢ are independently selected from hydrogen deuterium, alkoxy, halogen or carboxyl. In some embodiments, R¹ is lower alkyl, R² is phenyl, R³ is alkoxy, R⁴ is cycloalkyl or cycloamino, Rₐ and R_{d} are independently selected from hydrogen, nitro or amino, R_{b} and R_{c} are independently selected from hydrogen, halogen, hydroxyl or alkoxy, Rₑ, R_{f}, Rₕ and Rᵢ are independently selected from hydrogen or deuterium, and/or R_{g} is halogen, alkoxy or carboxyl. In some embodiments, R¹ is methyl, R² is phenyl, R³ is ethoxy, R⁴ is cycloamino or cycloalkyl, Rₐ and R_{d} are independently hydrogen, nitro or amino, R_{c} and R_{d} are hydrogen, Rₑ, R_{f}, Rₕ and Rᵢ are independently selected from hydrogen or deuterium, and/or R_{g} is halogen.

In some embodiments, A is C(R_{X}), [symbol] is C(Rₐ)═C(R_{b})—C(R_{c})═C(R_{d}), R_{X} is lower alkyl, cycloalkylalkyl or R¹—R²—R³—R⁴, R¹ is lower alkyl or carbonyl, R² absent or phenyl, R³ is lower alkyl, alkoxy or absent, R⁴ is cycloalkyl or cycloamino, Rₐ, R_{b}, R_{c} and R_{d} are independently selected from hydrogen, halogen, hydroxyl or alkoxy, and/or Rₑ, R_{f}, R_{g}, Rₕ and Rᵢ are independently selected from hydrogen deuterium, hydroxyl, alkoxy, halogen or carboxyl. In some embodiments, R¹ is lower alkyl, R² is phenyl, R³ is alkoxy, R⁴ is cycloalkyl or cycloamino, Rₐ and R_{d} are hydrogen R_{b} and R_{c} are independently selected from hydrogen, halogen, hydroxyl, or alkoxy, and/or Rₑ, R_{f}, R_{g}, Rₕ and Rᵢ are independently selected from hydrogen deuterium, hydroxyl, alkoxy or carboxyl. In some embodiments, R_{X} is cycloalkylalkyl, R¹ is methyl, R² is phenyl, R³ is ethoxy, R⁴ is cycloalkyl, Rₐ and R_{d} are hydrogen, R_{c} and R_{d} are independently hydrogen or halogen, Rₑ, R_{f}, Rₕ and Rᵢ are independently selected from hydrogen or deuterium, and/or R_{g} is halogen or carboxyl.

In some embodiments, A is C(R_{X}), [symbol] is C(Rₐ)═C(R_{b})—C(R_{c})═C(R_{d}), R_{X} is cycloalkylalkoxyphenylalkyl, R¹ is lower alkyl or carbonyl, R² absent or phenyl, R³ is lower alkyl, alkoxy or absent, R⁴ is cycloalkyl or cycloamino or absent, Rₐ, R_{b}, R_{c} and R_{d} are independently selected from hydrogen, halogen, hydroxyl or alkoxy, and/or Rₑ, R_{f}, R_{g}, Rₕ and Rᵢ are independently selected from hydrogen deuterium, hydroxyl alkoxy, halogen or carboxyl. In some embodiments, R¹ is lower alkyl, R² is absent, R³ is absent, R⁴ is absent, Rₐ and R_{d} are hydrogen R_{b} and R_{c} are independently selected from hydrogen, halogen, hydroxyl, or alkoxy, and Rₑ, R_{f}, R_{g}, Rₕ and Rᵢ are independently selected from hydrogen deuterium, hydroxyl, alkoxy or carboxyl. In some embodiments, R_{X} is cycloalkylalkoxyphenylalkyl, R¹ is methyl, R² absent, R³ is absent, R⁴ is absent, Rₐ and R_{d} are hydrogen, R_{b} and R_{c} are independently selected from hydrogen, halogen or alkoxy, Rₑ, R_{f}, Rₕ and Rᵢ are independently selected from hydrogen or deuterium, and R_{g} is halogen or alkoxy.

In some embodiments, the compound is a compound having a chemical structure listed in Table 1. Also disclosed herein are compositions comprising any one or more of the compounds disclosed herein, or a pharmaceutically acceptable salt, ester, solvate, stereoisomer, tautomer, or prodrug of thereof. The compound and the composition can be for use in preventing, delaying the onset of, or treating an infection or a disease caused by a RNA virus, or for use in preventing, delaying the onset of, or treating an inflammatory effect of an infection or a disease caused by a RNA virus. The inflammatory effect can comprise, e.g., respiratory failure, a sequela of respiratory failure, acute lung injury, or acute respiratory distress syndrome, optionally the sequela of respiratory failure comprises multi-organ failure. In some embodiments, inflammatory effect comprise acute hepatitis, chronic hepatitis, liver swelling, liver damage, joint inflammation, muscle pain and weakness, or blood vessel problem.

The composition can comprise, e.g., a therapeutically or prophylactically effective amount of the compound, or a pharmaceutically acceptable salt, ester, solvate, stereoisomer, tautomer, or prodrug of any one or more of the compounds disclosed herein (e.g., pipendoxifene and the compounds provided in Table 1 herein). The RNA virus can be, a double-stranded RNA virus, a negative-sense single-stranded RNA virus, or a positive-sense single-stranded RNA virus. The negative-sense single-stranded RNA virus can be, e.g., an influenza virus, Ebola virus, hantaviruses, Lassa fever virus, or rabies virus; and optionally the influenza virus is an influenza A virus, influenza B virus, influenza C virus, or influenza D virus. In some embodiments, the negative-sense single-stranded RNA virus is a virus in Orthornavirae. In some embodiments, the positive-sense single-stranded RNA virus is a virus in Caliciviridae. Flaviviridae or Coronaviridae. In some embodiments, the positive-sense single-stranded RNA virus is a coronavirus or norovirus, optionally the coronavirus is an alpha coronavirus, a beta coronavirus, a gamma coronavirus, a delta coronavirus, or an Omicron coronavirus. In some embodiments, the coronavirus is human coronavirus 229E (HCoV-229E), human coronavirus NL63 (HCoV-NL63), human coronavirus OC43 (HCoV—OC43), human coronavirus HKU1 (HCoV—HKU1), Middle East respiratory coronavirus (MERS-CoV), severe acute respiratory syndrome coronavirus (SARS-CoV), or SARS-CoV-2. The positive-sense single-stranded RNA virus can be a hepatitis C virus, e.g., a genotype 1, genotype 2, genotype 3, genotype 4, genotype 5, or genotype 6 hepatitis C virus.

In some embodiments, the composition is a pharmaceutical composition comprising the compound or a pharmaceutically acceptable salt, ester, solvate, stereoisomer, tautomer, or prodrug thereof, and one or more pharmaceutically acceptable excipients. The composition can comprise one or more additional therapeutic agents, optionally the one or more additional therapeutic agents comprise one or more antiviral agents. The one or more antiviral agents can be, e.g., a nucleoside, a non-nucleoside analogue reverse-transcriptase inhibitor, a nucleotide analogue reverse-transcriptase inhibitor, a NS3/4A serine protease inhibitor, a NS5B polymerase inhibitor, or interferon alpha.

The composition can be, e.g., in the form of powder, pill, tablet, microtablet, pellet, micropellet, capsule, capsule containing microtablets, liquid, aerosols, or nanoparticles. In some embodiments, the composition is in a formulation for administration to the lungs. In some embodiments, the composition is in a formulation for oral or intravenous administration.

Disclosed herein includes a method for preventing, delaying the onset of, or treating an infection or a disease caused by a RNA virus, comprising administering to a subject in need thereof any of the compounds disclosed herein or any of the compositions disclosed herein, thereby preventing, delaying the onset of, or treating the infection or the disease. Disclosed herein includes a method for preventing, delaying the onset of, or treating an inflammatory effect of an infection or a disease caused by a RNA virus, comprising administering to a subject in need thereof any of the compounds disclose herein or any of the compositions disclosed herein, thereby preventing, delaying the onset of, or treating the inflammatory effect. Disclosed herein includes a method for preventing, delaying the onset of, or treating an infection or a disease caused by a RNA virus, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease, and wherein the RNA virus is not a coronavirus. Also disclosed herein includes a method for preventing, delaying the onset of, or treating an inflammatory effect of an infection or a disease caused by a RNA virus, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect, and wherein the RNA virus is not a coronavirus.

In some embodiments, the inflammatory effect comprises respiratory failure, a sequela of respiratory failure, acute lung injury, or acute respiratory distress syndrome, optionally the sequela of respiratory failure comprises multi-organ failure. In some embodiments, the inflammatory effect comprise acute hepatitis, chronic hepatitis, liver swelling, liver damage, joint inflammation, muscle pain and weakness, or blood vessel problem.

The RNA virus can be, e.g., a double-stranded RNA virus, a negative-sense single-stranded RNA virus, or a positive-sense single-stranded RNA virus. In some embodiments, the negative-sense single-stranded RNA virus is an influenza virus, Ebola virus, hantaviruses, Lassa fever virus, and the rabies virus, and optionally the influenza virus is an influenza A virus, influenza B virus, influenza C virus, or influenza D virus. In some embodiments, the negative-sense single-stranded RNA virus is a virus in Orthornavirae. In some embodiments, the positive-sense single-stranded RNA virus is a virus in Caliciviridae. Flaviviridae or Coronaviridae. In some embodiments, the RNA virus is a coronavirus or norovirus, optionally the coronavirus is an alpha coronavirus, a beta coronavirus, a gamma coronavirus, or a delta coronavirus. In some embodiments, the coronavirus is human coronavirus 229E (HCoV-229E), human coronavirus NL63 (HCoV-NL63), human coronavirus OC43 (HCoV—OC43), human coronavirus HKU1 (HCoV—HKU1), Middle East respiratory coronavirus (MERS-CoV), severe acute respiratory syndrome coronavirus (SARS-CoV), or SARS-CoV-2. In some embodiments, the positive-sense single-stranded RNA virus is a hepatitis C virus. In some embodiments, the infection or disease caused by the RNA virus is common cold, flu, SARS, liver diseases, West Nile fever, Ebola virus disease, rabies, polio, measles, or a combination thereof.

The composition can comprise a therapeutically or prophylactically effective amount of the compound. In some embodiments, the subject in need thereof is a subject that is suffering from the infection or the disease, or a subject that is at a risk for the infection or the disease. In some embodiments, the infection or the disease is in the respiratory tract of the subject. In some embodiments, the infection or the disease is in the liver of the subject. In some embodiments, the subject is a subject that has been exposed to the RNA virus, is suspected to have been exposed to the RNA virus, or is at a risk of being exposed to the RNA virus. The subject can be, e.g., a mammal, optionally the subject is a human.

In some embodiments, the method can comprise administering to the subject one or more additional antiviral agents. At least one of the one or more additional antiviral agents can be co-administered to the subject with the composition. In some embodiments, at least one of the one or more additional antiviral agents is administered to the subject before the administration of the composition, after the administration of the composition, or both.

In some embodiments, the composition comprises one or more additional therapeutic agents, e.g., one or more antiviral agents (e.g., a nucleoside or a non-nucleoside analogue reverse-transcriptase inhibitor, a nucleotide analogue reverse-transcriptase inhibitor, a NS3/4A serine protease inhibitor, a NS5B polymerase inhibitor, or interferon alpha).

In some embodiments, the composition is administered to the subject by intravenous administration, nasal administration, pulmonary administration, oral administration, parenteral administration, or nebulization. In some embodiments, the composition is aspirated into at least one lung of the subject. In some embodiments, the composition is in the form of powder, pill, tablet, microtablet, pellet, micropellet, capsule (including capsule containing microtablets), liquid, aerosols, or nanoparticles. In some embodiments, the composition is in a formulation for administration to the lungs. In some embodiments, the composition is in a formulation for oral or intravenous administration.

The composition can be, e.g., administered to the subject once, twice, or three times a day. The composition can be, e.g., administered to the subject once every day, every two days, or every three days. In some embodiments, the composition is administered to the subject over the course of at least two weeks, at least three weeks, at least four weeks, or at least five weeks.

In some embodiments, the method further comprises measuring the viral titer of the RNA virus in the subject before administering the composition to the subject, after administering the composition to the subject, or both, optionally the viral titer is lung bulk virus titer. In some embodiments, the method comprises administrating the composition results in reduction of the viral titer of the RNA virus in the subject as compared to that in the subject before administration of the composition. In some embodiments, the method further comprises determining global virus distribution in an organ or a tissue of the subject; and optionally the method comprises determining global virus distribution in lungs of the subject.

In some embodiments, the method comprises measuring a neutrophil density within the lungs of the subject, optionally administering the composition results in reduction of the neutrophil density within the lungs of the subject as compared to that in the subject before administration of the composition. In some embodiments, the method comprises measuring a total necrotized cell count within the lungs of the subject, optionally administering the composition results in reduction of the total necrotized cell count in the subject as compared to that in the subject before administration of the composition. In some embodiments, the method comprises measuring a total protein level within the lungs of the subject, optionally administering the composition results in reduction of the total protein level within the lungs of the subject as compared to that in the subject before administration of the composition. In some embodiments, the method comprises measuring liver enzyme levels in the blood of the subject, optionally the liver enzymes comprise alanine aminotransferase (ALT) and aspartate aminotransferase (AST), and further optionally administering the composition results in reduction of the liver enzyme levels in the blood of the subject as compared to that in the subject before administration of the composition. In some embodiments, the method comprises measuring albumin level in the blood of the subject, optionally administering the composition results in increase of the albumin level in the blood of the subject as compared to that in the subject before administration of the composition. In some embodiments, the method comprises measuring HCV RNA and/or anti-HCV antibody in the blood of the subject, optionally administering the composition results in reduction of the HCV RNA and/or anti-HCV antibody in the subject as compared to that in the subject before administration of the composition.

Disclosed herein includes a kit. The kit can, in some embodiments, comprises one or more of the compounds disclosed herein (e.g., the compounds provided in Table 1), or a pharmaceutically acceptable salt, ester, solvate, stereoisomer, tautomer, or prodrug thereof; and a label indicating that the kit is for preventing, delaying the onset of, or treating an infection or a disease caused by a RNA virus; or a label indicating that the kit is for preventing, delaying the onset of, or treating an inflammatory effect of an infection or a disease caused by a RNA virus. In some embodiments, the kit comprises pipendoxifene or a pharmaceutically acceptable salt, ester, solvate, stereoisomer, tautomer, or prodrug thereof, and a label indicating that the kit is for preventing, delaying the onset of, or treating an infection or a disease caused by a RNA virus; or a label indicating that the kit is for preventing, delaying the onset of, or treating an inflammatory effect of an infection or a disease caused by a RNA virus; wherein the RNA virus is not a coronavirus. The RNA virus can be, e.g., a positive-sense single-stranded RNA virus. In some embodiments, the RNA virus is coronavirus, norovirus or hepatitis C virus. In some embodiments, the RNA virus is hepatitis C virus or norovirus. The RNA virus can be, e.g., a negative-sense single-stranded RNA virus. The RNA virus can be, e.g., a double-stranded RNA virus

### BRIEF DESCRIPTION OF THE DRAWINGS

FIG. 1 depicts a family tree diagram of 4 viral families, 13 viruses and variants for which pipendoxifene exhibited sub 10 μM antiviral activity. The best EC50 values obtained for each virus/strain/variant obtained across 29 assays are plotted. Values converted to account for differences in FBS concentrations are indicated in blue (the 11 values for WA1, Alpha, Beta, Delta, Omicron, MA, 229E α hCoV, OC443 β hCoV, Type 1b Hep C, NorwalkNoro, and WSN/33 H1N1). Complete results including SI50 calculations can be found in Table 3. Pipendoxifene Lung, Cₘₐₓ concentrations in mice after Multiple Dose (MD) and Single Dose (SD) of 250 mg/kg QD dosing as measured in PK studies are shown in the figure, respectively.

FIG. 2 depicts in vivo antiviral efficacy of pipendoxifene in mice infected with MA-SARS-CoV-2 (A) 129/S mice were intranasally infected with 2.5×10⁴ PFU of MA-SARS-CoV-2 and treated orally with pipendoxifene or subcutaneously with 100 mg/kg remdesivir twice daily for 3 days. Animal weights were monitored daily. N=9 per group. (B) SARS-CoV-2 titers in the lungs were determined on day 3 post-infection. N=9. (A-B) tested by two-way ANOVA (*P<0.05, **P<0.01, ***P<0.001, and ****P<0.0001).

FIG. 3 depicts a non-limiting exemplary experimental workflow used in the present disclosure.

FIG. 4 depicts non-limiting exemplary data related to chemical space analysis of exemplary compounds. A) Label names, color code, and symbols assigned to the data visualized with PCA and t-SNE. Dataset composition and label logic is explained in materials and methods. B) Molecular structures of MDL-001 (Pipendoxifene) and Beclabuvir; C) PCA where the first two principal components explain 24% of the total variance in the data and D) t-SNE of the chemical space for broad-spectrum antiviral ligands. The rectangular inset in panels C and D, show the chemical space covered by the new chemical entities (NCEs), pipendoxifene, Beclabuvir in comparison to the curated datasets. These plots highlight that the NCEs occupy a distinct chemical space bridging ‘explicit’ and ‘implicit’ RNA-dependent RNA polymerase (RdRp) inhibitors, demonstrating their novelty and alignment with the intended MoA.

FIG. 5 depicts poses of pipendoxifene (A) and beclabuvir (B) docked into the Thumb-1 pocket of HCV RdRp acquired from PDB ID: 4DRU [35]. (C-D) pipendoxifene (C) and beclabuvir (D) docked into the Thumb-1 pocket of SARS-CoV-2 RdRp acquired from PDB ID: 6M71 [23].

FIG. 6 depicts a graph showing tanimoto coefficients of exemplary new chemical entities (NCEs). The grouped bar plot in this figure shows the Tanimoto coefficients calculated for the 12 NCEs (Compounds 1-12 provided in Table 1), when compared to MDL-001 (pipendoxifene light blue bars on the left) and Beclanbuvir (orange bars on the right). Small molecules were embedded in the ECFP4 format for Tanimoto coefficient calculation. The dashed, horizontal line (colored red) indicates the chemical novelty threshold (Tanimoto coefficient≥0.5).

FIG. 7 depicts non-limiting exemplary data related to in vito antiviral assays. Shown in this figure are the EC₅₀ values obtained for 12 NCEs (Compounds 1-12 provided in Table 1) and MDL-001 (pipendoxifene), when tested against pseudovirus inhibitory assays specific to HCV 1b (blue circles) and CoV229E (orange squares). EC₅₀ values are plotted in M units along the y-axis.

FIG. 8 depicts non-limiting exemplary data related to MCF7 growth inhibition assays. The plot in this figure shows the half-maximal inhibitory concentration (IC₅₀) values for 12 NCEs (Compounds 1-12 provided in Table 1) and MDL-001 (pipendoxifene), when added to MCF7 cells. IC₅₀ values are plotted along the left y-axis. The relative NCE IC₅₀ with respect to MDL-001's is plotted along the right y-axis.

FIG. 9 depict non-limiting exemplary data related to AUROC and binary cross-entropy loss for t-SNE split training and validation sets (broad-spectrum model).

FIG. 10 depict non-limiting exemplary data related to AUROC and binary cross-entropy loss for t-SNE split training and validation sets (ER binding model).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

The methods, compounds, pharmaceutical compositions and articles of manufacture provided herein are characterized by a variety of component ingredients, steps of preparation, and steps of execution and associated biophysical, physical, biochemical or chemical parameters. As would be apparent to those of skill in the art, the methods provided herein can include any and all permutations and combinations of the compounds, compositions, articles of manufacture and associated ingredients, steps and/or parameters as described below.

Disclosed herein include broad-spectrum antiviral compounds (including compounds of Formula I) and pharmaceutically acceptable salts, ester, solvate, stereoisomer, tautomer, or prodrug thereof:

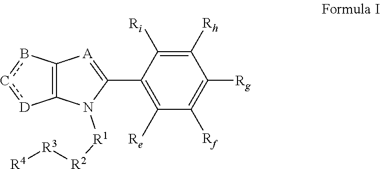

wherein A is independently:
   1) Nitrogen; or
   2) —C(R_{X})
[symbol] is:
   1) C(Rₐ)═C(R_{b})—C(R_{c})═C(R_{d}); or
   2) S—C(R_{c})═C(R_{d})
R¹ is:
   1) Lower alkyl;
   2) Haloalkyl;
   3) Cycloalkylalkyl; or
   4) Carbonyl
R² is:
   1) Phenyl;
   2) Cycloalkyl; or
   3) Absent
R³ is:
   1) Lower alkyl;
   2) Alkoxy; or
   3) Absent
R⁴ is:
   1) Cyclalkyl;
   2) Cycloamine; or
   3) Absent
R_{X} is:
   1) Hydrogen
   2) Deuterium
   3) Halogen
   4) Lower alkyl
   5) Haloalkyl;
   6) Cycloalkyl;
   7) Cycloalkylalkyl; or
   8) R¹—R²—R³—R⁴
Rₐ, R_{b}, R_{c}, and R_{d} are independently selected from:
   1) Hydrogen;
   2) Deuterium;
   3) Alkoxy;
   4) Halogen;
   5) Hydroxy
   6) Nitro; or
   7) Amino; and
Rₑ, R_{f}, R_{g}, Rₕ and Rᵢ are independently selected from:
   1) Hydrogen;
   2) Deuterium
   3) Alkoxy;
   4) Halogen
   5) Nitro;
   6) Carboxyl; or
   7) Carboxylic ester.

In some embodiments, the compound is a compound having a chemical structure provided in Table 1.

Disclosed herein also include compositions. In some embodiments, the composition comprises the compound from Table 1 or a pharmaceutically acceptable salt, ester, solvate, stereoisomer, tautomer, or prodrug thereof, for use in preventing, delaying the onset of, or treating an infection or a disease caused by a RNA virus. In some embodiments, the composition comprises the compound from Table 1 or a pharmaceutically acceptable salt, ester, solvate, stereoisomer, tautomer, or prodrug thereof, for use in preventing, delaying the onset of, or treating an inflammatory effect of an infection or a disease caused by a RNA virus.

Disclosed herein also include methods for preventing, delaying the onset of, or treating an infection or a disease caused by a RNA virus. In some embodiments, the method comprises administrating to a subject in need thereof a composition comprising a compound disclosed herein (e.g., a compound from Table 1) or a pharmaceutically acceptable salt, ester, solvate, stereoisomer, tautomer, or prodrug thereof, thereby preventing, delaying the onset of, or treating the infection or the disease. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease. In some embodiments, the method comprises to a subject in need thereof (1) a first compound from Table 1, or a pharmaceutically salt, ester, solvate, stereoisomer, tautomer, or prodrug thereof, and (2) a second compound from Table 1 or pipendoxifene or a pharmaceutically salt, ester, solvate, stereoisomer, tautomer, or prodrug thereof, thereby preventing, delaying the onset of, or treating the infection or the disease caused by a RNA virus, wherein the first compound and the second compound are different.

Disclosed herein also include methods for preventing, delaying the onset of, or treating an inflammatory effect of an infection or a disease caused by a RNA virus. In some embodiments, the method comprises administrating to a subject in need thereof a composition comprising a compound disclosed herein (e.g., a compound from Table 1) or a pharmaceutically acceptable salt, ester, solvate, stereoisomer, tautomer, or prodrug thereof, thereby preventing, delaying the onset of, or treating the inflammatory effect. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect. In some embodiments, the method comprises administering to a subject in need thereof (1) a first compound from Table 1, or a pharmaceutically salt, ester, solvate, stereoisomer, tautomer, or prodrug thereof, and (2) a second compound from Table 1 or pipendoxifene or a pharmaceutically salt, ester, solvate, stereoisomer, tautomer, or prodrug thereof, thereby preventing, delaying the onset of, or treating the inflammatory effect, wherein the first compound and the second compound are different.

Disclosed herein also includes kits. In some embodiments, the kit comprises a first compound or a pharmaceutically acceptable salt, ester, solvate, stereoisomer, tautomer, or prodrug thereof, wherein the first compound is a compound disclosed herein (e.g., a compound listed in Table 1); and a label indicating that the kit is for preventing, delaying the onset of, or treating an infection or a disease caused by a RNA virus. In some embodiments, the kit comprises a first compound or a pharmaceutically acceptable salt, ester, solvate, stereoisomer, tautomer, or prodrug thereof. The first compound can be any of the compounds disclose herein (e.g., a compound listed in Table 1) and a label indicating that the kit is for preventing, delaying the onset of, or treating an inflammatory effect of an infection or a disease caused by a RNA virus.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, “compound”, “compounds”, “chemical entity”, and “chemical entities” refer to a compound encompassed by the generic formula disclosed herein, any subgenus of those generic formula, and any forms of the compounds within the generic and subgeneric formula, including the pharmaceutically acceptable salts, esters, solvates, racemates, stereoisomers, tautomers, and prodrugs of the compound or compounds.

As used herein, “alkyl” refers to an unbroken non-cyclic chain of carbon atoms that may be unsubstituted or substituted with deuterium or halogen. It may also be branched or unbranched.

As used herein, “lower alkyl” refers to a branched or straight chain acyclic alkyl group comprising one to ten carbon atoms, preferably one to eight carbon atoms, more preferably one to six carbon atoms which may be substituted with one or more deuterium. Exemplary lower alkyl groups include methyl, trideuteromethyl, ethyl, pentadeuteroethyl, n-propyl, isopropyl, n-butyl, isobutyl, sec-butyl, t-butyl, pentyl, neopentyl, iso-amyl, hexyl, and octyl.

As used herein, “haloalkyl” refers to a lower alkyl group a cycloalkyl group or a heterocyclic ring as defined herein, to which is appended one or more halogens, as defined herein. Exemplary haloalkyl groups include trifluoromethyl, chloromethyl, 2-bromobutyl, 1-bromo-2-chloro-pentyl, 4,4-difluorocyclohexyl, 2-(tetrahydro-2H-pyran-4-yl).

As used herein, “cycloalkyl” refers to a saturated or unsaturated cyclic hydrocarbon comprising from about 3 to about 10 carbon atoms. Cycloalkyl groups can be unsubstituted or substituted with one or more substituents independently selected from deuterium, lower alkyl, haloalkyl, alkoxy, amino, alkylamino, dialkylamino, amidyl, ester, hydroxy, halo, carboxyl, alkylcarboxylic acid, alkylcarboxylic ester, carboxamido and alkylcarboxamido. Exemplary cycloalkyl groups include cyclopropyl, cyclobutyl, cyclopentyl, cyclohexyl, cyclohexenyl, cyclohepta-1,3-dienyl and 4-cyclohexanonyl.

As used herein, “phenyl” refers to a benzene ring which can be substituted with one, two, three or four substituents selected from deuterium, lower alkyl, haloalkyl, alkoxy, amino, alkylamino, dialkylamino, amidyl, ester, hydroxy, halo and carboxyl. Exemplary phenyl groups include 1-ethoxy-4-methylbenzene, 1-isopropoxy-4-methylbenzene and N-(4-ethylphenyl)acetamide.

As used herein, “cycloalkylalkyl” refers to a cycloalkyl radical, as defined herein, attached to an alkyl radical, as defined herein. Exemplary cycloalkylalkyl groups include methylcyclopentane, methylcyclopropane and ethylcyclobutane.

As used herein, “heterocyclic ring or group” refers to a saturated or unsaturated cyclic or polycyclic hydrocarbon group having about 2 to about 12 carbon atoms where 1 to about 4 carbon atoms are replaced by one or more nitrogen, oxygen and/or sulfur atoms. Sulfur may be in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, hydroxy, oxo, halo, carboxyl, carboxylic ester, aryl, amidyl, carboxylic ester, carboxamido, and nitro. Exemplary heterocyclic groups include pyrrolyl, furyl, thienyl, 3-pyrrolinyl,4,5,6-trihydro-2H-pyranyl, pyridinyl, 1,4-dihydropyridinyl, pyrazolyl, triazolyl, pyrimidinyl, pyridazinyl, oxazolyl, thiazolyl, thieno[2,3-d]pyrimidine, 4,5,6,7-tetrahydrobenzo[b]thiophene, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl and 2,6-dioxabicyclo(3.3.0)octane.

As used herein, “carbonyl” refers to —C(O)—.

As used herein, “alkoxy” refers to R₅₀O—, wherein R₅₀ is an alkyl group, an alkenyl group or an alkynyl group as defined herein (preferably a lower alkyl group or a haloalkyl group, as defined herein). Exemplary alkoxy groups include methoxy, ethoxy, t-butoxy, cyclopentyloxy, trifluoromethoxy, propenyloxy and propargyloxy.

As used herein, “cycloamine” refers to a three, four, five, six, seven or eight membered ring containing at least one basic nitrogen which is attached via the amine nitrogen to an alkyl radical that connects to additional functional groups. Exemplary cyclaminoalkyl groups include 1-methylpiperidine and 1-methylazepine.

As used herein, “cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, ester, hydroxy, halo, carboxyl, carboxamido, oxo, and nitro. Exemplary cycloalkyl groups include cyclopropyl, cyclobutyl, cyclopentyl, cyclohexyl, cyclohexenyl and cyclohepta-1,3-dienyl.

As used herein, “cycloalkylalkyl” refers to a cycloalkyl radical, as defined herein, attached to an alkyl radical, as defined herein. Exemplary cycloalkylalkyl groups include methylcyclopentane and ethylcyclobutane.

As used herein, “haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl and 1-bromo-2-chloro-pentyl.

As used herein, “halogen” or “halo” refers to iodine (I), bromine (Br), chlorine (Cl), and/or fluorine (F).

As used herein, “nitro” refers to the group —NO₂.

As used herein, “hydroxy” refers to —OH.

As used herein, “nitrile” and “cyano” refer to —CN

As used herein, “alkoxy” refers to R₅₀O—, wherein R₅₀ is an alkyl group, an alkenyl group or an alkynyl group as defined herein (preferably a lower alkyl group or a haloalkyl group, as defined herein). Exemplary alkoxy groups include methoxy, ethoxy, t-butoxy, cyclopentyloxy, trifluoromethoxy, propenyloxy and propargyloxy.

As used herein, “amino” refers to —NH₂, an alkylamino group or a dialkylamino group as defined herein.

As used herein, “alkylamino” refers to R₅₀NH—, wherein R₅₀ is an alkyl group, as defined herein. Exemplary alkylamino groups include methylamino, ethylamino, butylamino, and cyclohexylamino.

As used herein, “dialkylamino” refers to R₅₂R₅₃N—, wherein R₅₂ and R₅₃ are each independently an alkyl group, as defined herein. Exemplary dialkylamino groups include dimethylamino, diethylamino and methyl propargylamino.

As used herein, “arylamino” refers to R₅₅NH—, wherein R₅₅ is an aryl group, as defined elsewhere herein.

As used herein, “diarylamino” refers to R₅₅R₆₀N—, wherein R₅₅ and R₆₀ are each independently an aryl group, as defined herein.

As used herein, “alkylarylamino refers to R₅₀R₅₅N—, wherein R₅₀ is an alkyl group and R₅₅ is an aryl group, as defined herein.

As used herein, “carboxyl” refers to —C(O)OR₇₆, wherein R₇₆ is a hydrogen, an organic cation or an inorganic cation, as defined herein.

As used herein, “carboxylic ester” refers to —C(O)OR₅₅, wherein R₅₅ is an alkyl group or an aryl group, as defined herein.

As used herein, “amidyl” refers to R₅₁C(O)N(R₅₇)— wherein R₅₁ and R₅₇ are each independently a hydrogen atom, an alkyl group or an aryl group as defined herein.

As used herein, “carboxamido” refers to —C(O)N(R₅₁)(R₅₇), wherein R₅₁ and R₅₇ are each independently a hydrogen atom, an alkyl group or an aryl group, as defined herein, or R₅₁ and R₅₇ when taken together form a cycloamine group, as defined herein.

As used herein, “aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl. Aryl groups (including bicyclic aryl groups) can be unsubstituted or substituted with one, two or three substituents independently selected from alkyl, alkoxy, amino, alkylamino, dialkylamino, arylamino, diarylamino, alkylarylamino, halo, cyano, hydroxy, carboxyl, carboxylic ester, and nitro. Exemplary substituted aryl groups include tetrafluorophenyl, pentafluorophenyl and 4-benzoic acid.

As used herein, “cycloalkylalkoxyphenylalkyl” refers to R₁₀₀—R₁₀₁—R₁₀₂—R₁₀₃— wherein R₁₀₀ is a cycloalkyl, as defined herein, R₁₀₁ is an alkoxy, as defined herein, R₁₀₂ is a phenyl, as defined herein and R₁₀₃ is a lower alkyl as defined herein. Exemplary cycloalkylalkoxyphenylalkyl groups include 1-(2-cyclohexylethoxy)-4-methylbenzene, (2-(4-methylphenoxy)ethyl)cycloheptane and (2-(2,3-difluoro-4-methylphenoxy)ethyl)cyclohexane.

As used herein, “solvate” or “solvates” of a compound refer to those compounds, as defined above, which are bound to a stoichiometric or non-stoichiometric amount of a solvent. Solvates of a compound includes solvates of all forms of the compound. In some embodiments, solvents are volatile, non-toxic, and/or acceptable for administration to humans in trace amounts. Suitable solvates include water.

As used herein, “racemates” refers to a mixture of enantiomers. In some embodiments, the compounds, or pharmaceutically acceptable salts thereof, can be enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

As used herein, “stereoisomer” or “stereoisomers” refer to compounds that differ in the chirality of one or more stereocenters. Stereoisomers include enantiomers and diastereomers.

As used herein, “tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable” as used herein means approved by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound disclosed herein is administered.

“Prodrug” refers to a derivative of a drug molecule that requires a transformation within the body to release the active drug. Prodrugs are frequently (though not necessarily) pharmacologically inactive until converted to the parent drug. Typically, prodrugs are designed to overcome pharmaceutical and/or pharmacokinetically based problems associated with the parent drug molecule that would otherwise limit the clinical usefulness of the drug.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo. Ideally, the promoiety is rapidly cleared from the body upon cleavage from the prodrug.

“Protecting group” refers to a grouping of atoms that when attached to a reactive group in a molecule masks, reduces or prevents that reactivity. Examples of protecting groups can be found in Green et al., “Protective Groups in Organic Chemistry”, (Wiley, 2.sup.nd ed. 1991) and Harrison et al., “Compendium of Synthetic Organic Methods”, Vols. 1 8 (John Wiley and Sons, 1971 1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e. Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to the combined amount of the active ingredients (e.g., berzosertib and/or pipendoxifene).

As used herein, “therapeutically effective amount” or “pharmaceutically effective amount” is meant an amount of therapeutic agent, which has a therapeutic effect. The dosages of a pharmaceutically active ingredient which are useful in treatment are therapeutically effective amounts. Thus, as used herein, a therapeutically effective amount means an amount of therapeutic agent which produces the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire). “Treat,” “treatment,” or “treating,” as used herein refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, EC₅₀ is the value of a graded dose response curve that represents the concentration of a compound where 50% of its maximal effect is observed.

As used herein, CC₅₀ is the 50% cytotoxic concentration defined as the compound's concentration (μg/mL) required for the reduction of cell viability by 50%.

As used herein, SI=CC₅₀/EC₅₀. The selectivity index (SI) is a ratio that measures the window between cytotoxicity and antiviral activity by dividing the CC₅₀ value into the EC₅₀ value. The higher the SI ratio, the theoretically more effective and safe a drug would be during in vivo treatment for a given viral infection.

“Individual” as used herein refers to a; person, human adult or child, mammal, or non-human primate.

“IC50” as used herein refers to the molar concentration of a compound, for example a compound from Table 1 or iipendoxifene, which binds 50% of receptor related to RNA viral infection in vitro.

“Ki” as used herein refers to the kinetic inhibition constant in molar concentration units which denotes the affinity of the compound from Table 1 or pipendoxifene for the receptor related to RNA viral infection as measured by a binding assay or as calculated from the IC50 value using the Cheng-Prusoff equation.

“Patient” as used herein refers to a mammal, e.g., a human, mouse, rat, guinea pig, dog, cat, horse, cow, pig, or non-human primate, such as a monkey, chimpanzee, baboon or rhesus.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Treating” or “treatment” of any disease or disorder as used herein, refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” as used herein, means the amount of a compound that, when administered to an individual for treating a disease, is sufficient to effect such treatment for the disease or to achieve the desired clinical response. The “therapeutically effective amount” will vary depending on the compound, the disease and its severity and the age, weight, etc., of the patient to be treated.

As used herein, a “dosage” refers to an amount of therapeutic agent administered to a patient.

As used herein, a “daily dosage” refers to the total amount of therapeutic agent administered to a patient in a day.

As used herein, the term “therapeutic agent” means a substance that is effective in the treatment of a disease or condition.

### Chemical Compounds

Provided herein are chemical compounds, related compositions and methods for the prevention or treatment of diseases or disorders related to RNA viral infections, for example, coronavirus infections and hepatitis virus infections. In some embodiments, the compounds disclosed herein prevent or treat diseases or disorders related to RNA viral infections through inhibition against RNA-dependent RNA polymerase (RdRp) and specifically its allosteric domain. In some embodiments, the compounds disclosed herein are broad-spectrum antiviral RdRp inhibitors. In some embodiments, the compounds disclosed herein are RdRp Thumb-1 inhibitors.

The compounds disclosed herein and compositions thereof can be used for viral load reduction and/or symptom reduction with activity against broad-spectrum viral infections such as coronavirus and hepatitis virus (e.g., HCV). As described herein, the compounds disclosed herein are designed using artificial intelligence (AI)-based drug discovery platform GALILEO and its geometric graph convolutional network tool ChemPrint. By integrating GALILEO's computational AI tools with downstream in vitro assays, new chemical entities are designed with desired broad-spectrum antiviral activity and enhanced binding specificity, and can be used to prevent and treat broad-spectrum viral infections including coronavirus and hepatitis virus infections.

Provided herein include broad spectrum antiviral compounds of Formula I, and pharmaceutically acceptable salts, ester, solvate, stereoisomer, tautomer, or prodrug thereof:

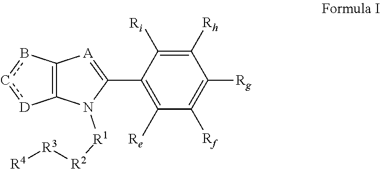

wherein A is independently:
   1) Nitrogen; or
   2) —C(R_{X})
[symbol] is:
   1) C(Rₐ)═C(R_{b})—C(R_{c})═C(R_{d}); or
   2) S—C(R_{c})═C(R_{d})
R¹ is:
   1) Lower alkyl;
   2) Haloalkyl;
   3) Cycloalkylalkyl; or
   4) Carbonyl
R² is:
   1) Phenyl;
   2) Cycloalkyl; or
   3) Absent
R³ is:
   1) Lower alkyl;
   2) Alkoxy; or
   3) Absent
R⁴ is:
   1) Cyclalkyl;
   2) Cycloamine; or
   3) Absent
R_{X} is:
   1) Hydrogen
   2) Deuterium
   3) Halogen
   4) Lower alkyl
   5) Haloalkyl;
   6) Cycloalkyl;
   7) Cycloalkylalkyl; or
   8) R¹—R²—R³—R⁴
Rₐ, R_{b}, R_{c}, and R_{d} are independently selected from:
   1) Hydrogen;
   2) Deuterium;
   3) Alkoxy;
   4) Halogen;
   5) Hydroxy
   6) Nitro; or
   7) Amino; and
Rₑ, R_{f}, R_{g}, Rₕ and Rᵢ are independently selected from:
   1) Hydrogen;
   2) Deuterium
   3) Alkoxy;
   4) Halogen
   5) Nitro;
   6) Carboxyl; or
   7) Carboxylic ester.

In some embodiments, the compound has the structure of Formula I, wherein A is CR_{X} or nitrogen, [symbol] is S—C(R_{c})═C(R_{d}) or C(Rₐ)═C(R_{b})—C(R_{c})═C(R_{d}), R_{X} is lower alkyl, haloalkyl or cycloalkylalkyl, R¹ is lower alkyl or carbonyl, R² or phenyl or absent, R³ is lower alkyl alkoxy or absent, R⁴ is cycloalkyl, cycloamino or absent, Rₐ, R_{b}, R_{c} and R_{d} are independently selected from hydrogen, halogen, hydroxyl, alkoxy, nitro or amino, and/or Rₑ, R_{f}, R_{g}, Rₕ and Rᵢ are independently selected from hydrogen, halogen, hydroxyl, alkoxy or carboxyl. R_{X} can be, e.g., hydrogen or lower alkyl.

In some embodiments, the compound has the structure of Formula I, wherein A is C(R_{X}) wherein R_{X} is hydrogen or lower alkyl, [symbol] is S—C(R_{c})═C(R_{d}), R¹ is lower alkyl, R² is phenyl, R³ is lower alkyl or alkoxy, R⁴ is cycloalkyl or cycloamino, R_{c} and R_{d} are hydrogen or deuterium, Rₑ, R_{f}, R_{g}, Rₕ and Rᵢ are independently selected from hydrogen, halogen, alkoxy or carboxyl. More preferable R_{X} is hydrogen, R¹ is lower alkyl, R² is phenyl, R³ is alkoxy, R⁴ is cycloalkyl or cycloamino, R_{c} and R_{d} are hydrogen, Rₑ, R_{f}, Rₕ and Rᵢ are independently selected from hydrogen or deuterium, R_{g} is hydrogen, deuterium or halogen. Most preferably R_{X} is hydrogen, R¹ is methyl, R² is phenyl, R³ is ethoxy, R⁴ is cycloamino, R_{c} and R_{d} are hydrogen, Rₑ, R_{f}, Rₕ and Rᵢ are independently selected from hydrogen or deuterium, and/or R_{g} is halogen.

In some embodiments, the compound has the structure of Formula I, wherein A is nitrogen, [symbol] is C(Rₐ)═C(R_{b})—C(R_{c})═C(R_{d}), R¹ is lower alkyl or carbonyl, R² absent or phenyl, R³ is lower alkyl or alkoxy, R⁴ is cycloalkyl or cycloamino, Rₐ, R_{b}, R_{c} and R_{d} are independently selected from hydrogen, halogen, hydroxyl, alkoxy, nitro or amino, and/or Rₑ, R_{f}, R_{g}, Rₕ and Rᵢ are independently selected from hydrogen deuterium, alkoxy, halogen or carboxyl. More preferable R¹ is lower alkyl, R² is phenyl, R³ is alkoxy, R⁴ is cycloalkyl or cycloamino, Rₐ and R_{d} are independently selected from hydrogen, nitro or amino, R_{b} and R_{c} are independently selected from hydrogen, halogen, hydroxyl or alkoxy, Rₑ, R_{f}, Rₕ and Rᵢ are independently selected from hydrogen or deuterium, and/or R_{g} is halogen, alkoxy or carboxyl. Most preferably R¹ is methyl, R² is phenyl, R³ is ethoxy, R⁴ is cycloamino or cycloalkyl, Rₐ and R_{d} are independently hydrogen, nitro or amino, R_{c} and R_{d} are hydrogen, Rₑ, R_{f}, Rₕ and Rᵢ are independently selected from hydrogen or deuterium, and/or R_{g} is halogen.

In some embodiments the compound has the structure of Formula I, wherein A is C(R_{X}), [symbol] is C(Rₐ)═C(R_{b})—C(R_{c})═C(R_{d}), R_{X} is lower alkyl, cycloalkylalkyl or R¹—R²—R³—R⁴, R¹ is lower alkyl or carbonyl, R² absent or phenyl, R³ is lower alkyl, alkoxy or absent, R⁴ is cycloalkyl or cycloamino, Rₐ, R_{b}, R_{c} and R_{d} are independently selected from hydrogen, halogen, hydroxyl or alkoxy, Rₑ, R_{f}, R_{g}, Rₕ and Rᵢ are independently selected from hydrogen deuterium, hydroxyl, alkoxy, halogen or carboxyl. More preferable R¹ is lower alkyl, R² is phenyl, R³ is alkoxy, R⁴ is cycloalkyl or cycloamino, Rₐ and R_{d} are hydrogen R_{b} and R_{c} are independently selected from hydrogen, halogen, hydroxyl, or alkoxy Rₑ, R_{f}, R_{g}, Rₕ and Rᵢ are independently selected from hydrogen deuterium, hydroxyl, alkoxy or carboxyl. Most preferably R_{X} is cycloalkylalkyl, R¹ is methyl, R² is phenyl, R³ is ethoxy, R⁴ is cycloalkyl, Rₐ and R_{d} are hydrogen, R_{c} and R_{d} are independently hydrogen or halogen, Rₑ, R_{f}, Rₕ and Rᵢ are independently selected from hydrogen or deuterium, and/or R_{g} is halogen or carboxyl.

In some embodiments, the compound has the structure of Formula I, wherein A is C(R_{X}), [symbol] is C(Rₐ)═C(R_{b})—C(R_{c})═C(R_{d}), R_{X} is cycloalkylalkoxyphenylalkyl, R¹ is lower alkyl or carbonyl, R² absent or phenyl, R³ is lower alkyl, alkoxy or absent, R⁴ is cycloalkyl or cycloamino or absent, Rₐ, R_{b}, R_{c} and R_{d} are independently selected from hydrogen, halogen, hydroxyl or alkoxy, Rₑ, R_{f}, R_{g}, Rₕ and Rᵢ are independently selected from hydrogen deuterium, hydroxyl alkoxy, halogen or carboxyl. More preferable R¹ is lower alkyl, R² is absent, R³ is absent, R⁴ is absent, Rₐ and R_{d} are hydrogen R_{b} and R_{c} are independently selected from hydrogen, halogen, hydroxyl, or alkoxy Rₑ, R_{f}, R_{g}, Rₕ and Rᵢ are independently selected from hydrogen deuterium, hydroxyl, alkoxy or carboxyl. Most preferably R_{X} cycloalkylalkoxyphenylalkyl, R¹ is methyl, R² absent, R³ is absent, R⁴ is absent, Rₐ and R_{d} are hydrogen, R_{b} and R_{c} are independently selected from hydrogen, halogen or alkoxy, Rₑ, R_{f}, Rₕ and Rᵢ are independently selected from hydrogen or deuterium, and/or R_{g} is halogen or alkoxy.

In some embodiment, the compounds disclosed herein is a compound having one of the chemical structures listed in Table 1. The compounds in Table 1 were designed using pipendoxifene's pharmacophoric scaffold as the training data while eliminating off-target binding.

TABLE 1
Exemplary compounds and structures

| Compound | Compound | |
|---|---|---|
| Name (MCAA) | Number¹ | Structure |
| MCAA-01 | N/A | 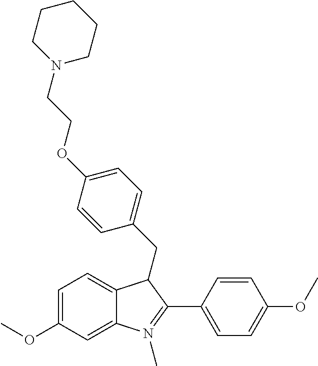 |
| | | Exact Mass: 484.27 |
| MCAA-02 | Compound 1 | 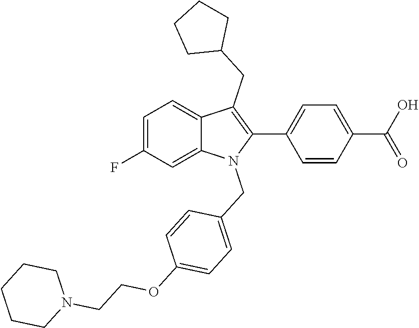 [SMILES: O=C(O)c1ccc(-c2c(CC3CCCC3)c3ccc(F)cc3n2Cc2ccc(OCCN3CCCCC3)cc2)cc1] |
| | | Exact Mass: 554.29 |
| MCAA-03 | Compound 2 | 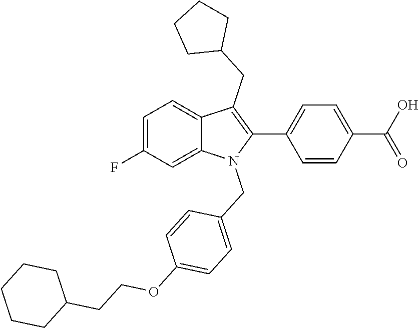 [SMILES: O=C(O)c1ccc(-c2c(CC3CCCC3)c3ccc(F)cc3n2Cc2ccc(OCCC3CCCCC3)cc2)cc1] |
| | | Exact Mass: 553.30 |
| MCAA-04 | N/A | 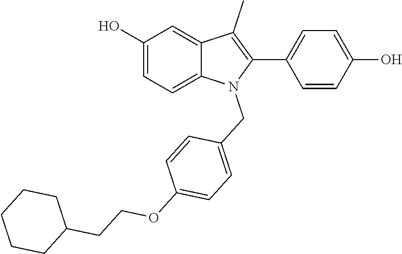 [SMILES: Cc1c(-c2ccc(O)cc2)n(Cc2ccc(OCCC3CCCCC3)cc2)c2ccc(O)cc12] |
| | | Exact Mass: 455.25 |
| MCAA-05 | N/A | 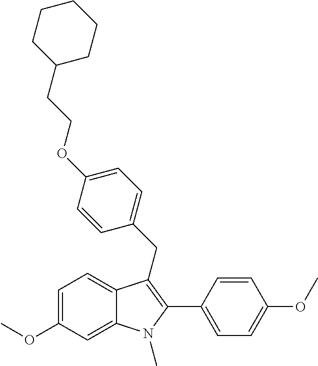 |
| | | Exact Mass: 483.28 |
| MCAA-06 | Compound 3 | 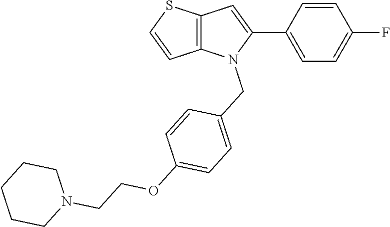 [SMILES: Fc1ccc(-c2cc3sccc3n2Cc2ccc(OCCN3CCCCC3)cc2)cc1] |
| | | Exact Mass: 434.18 |
| MCAA-07 | Compound 4 | 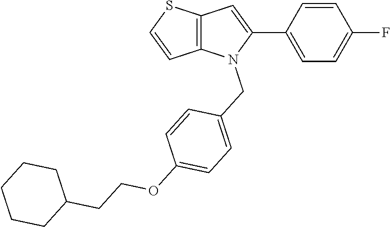 [SMILES: Fc1ccc(-c2cc3sccc3n2Cc2ccc(OCCC3CCCCC3)cc2)cc1] |
| | | Exact Mass: 433.19 |
| MCAA-08 | Compound 5 | 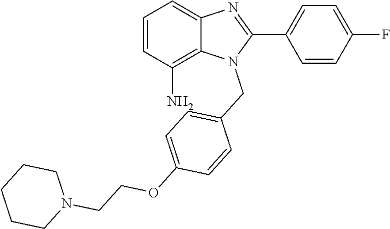 [SMILES: Nc1cccc2nc(-c3ccc(F)cc3)n(Cc3ccc(OCCN4CCCCC4)cc3)c12] |
| | | Exact Mass: 444.23 |
| MCAA-09 | Compound 6 | 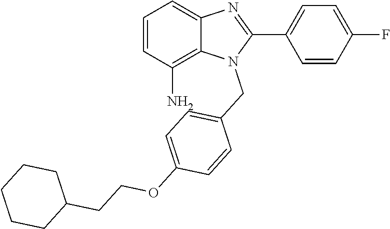 [SMILES: Nc1cccc2nc(-c3ccc(F)cc3)n(Cc3ccc(OCCC4CCCCC4)cc3)c12] |
| | | Exact Mass: 443.24 |
| MCAA-10 | Compound 7 | 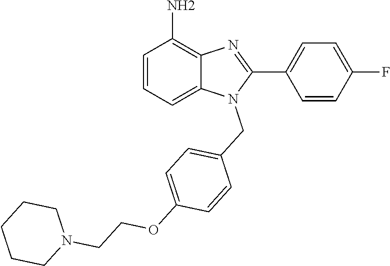 [SMILES: Nc1cccc2c1nc(-c1ccc(F)cc1)n2Cc1ccc(OCCN2CCCCC2)cc1] |
| | | Exact Mass: 444.23 |
| MCAA-11 | Compound 8 | 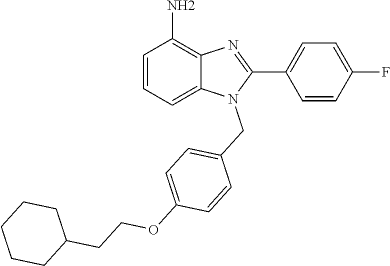 [SMILES: Nc1cccc2c1nc(-c1ccc(F)cc1)n2Cc1ccc(OCCC2CCCCC2)cc1] |
| | | Exact Mass: 443.24 |
| MCAA-12 | Compound 9 | 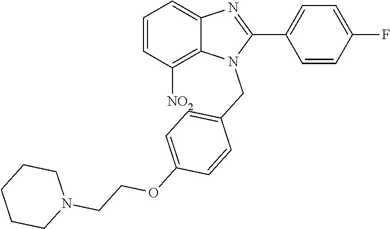 [SMILES: O=[N+]([O-])c1cccc2nc(-c3ccc(F)cc3)n(Cc3ccc(OCCN4CCCCC4)cc3)c12] |
| | | Exact Mass: 474.21 |
| MCAA-13 | Compound 10 | 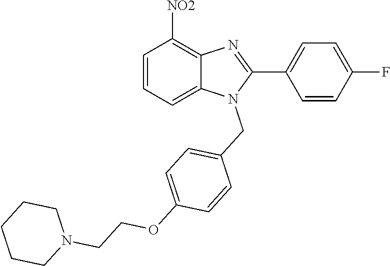 [SMILES: O=[N+]([O-])c1cccc2c1nc(-c1ccc(F)cc1)n2Cc1ccc(OCCN2CCCCC2)cc1] |
| | | Exact Mass: 474.21 |
| MCAA-14 | Compound 11 | 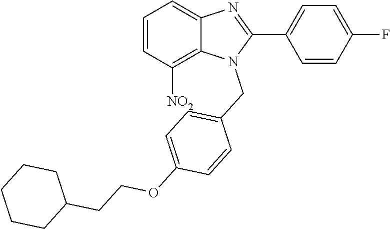 [SMILES: O=[N+]([O-])c1cccc2nc(-c3ccc(F)cc3)n(Cc3ccc(OCCC4CCCCC4)cc3)c12] |
| | | Exact Mass: 473.21 |
| MCAA-15 | Compound 12 | 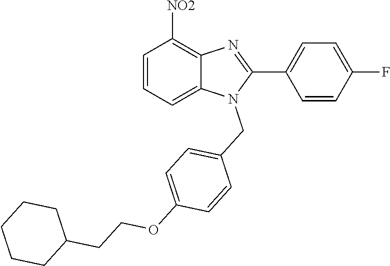 [SMILES: O=[N+]([O-])c1cccc2c1nc(-c1ccc(F)cc1)n2Cc1ccc(OCCC2CCCCC2)cc1] |
| | | Exact Mass: 473.21 |

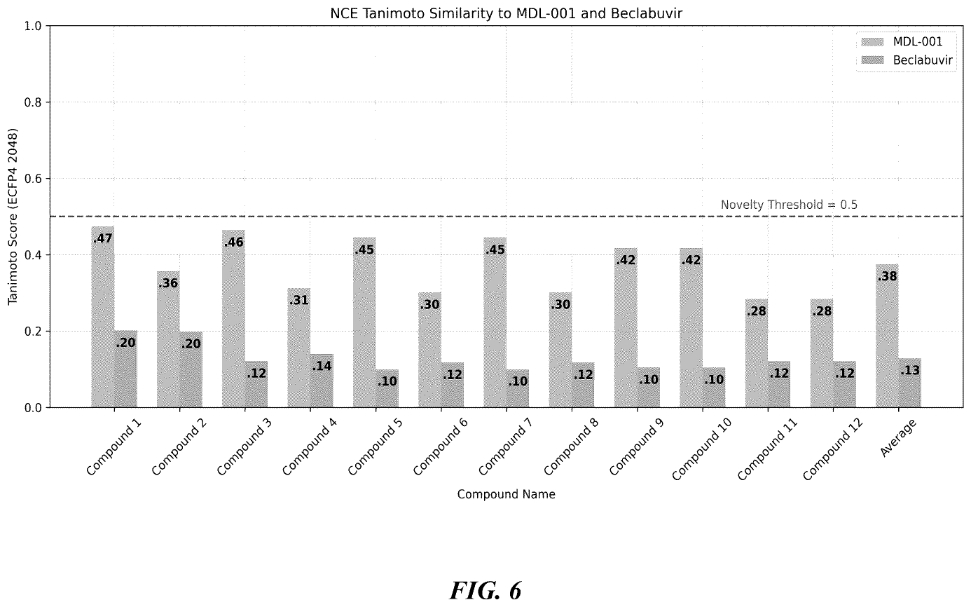

¹compound numbers used in FIGS. 6-8 Table 2.

In some embodiments, a compound used herein for preventing and treating disorders related to a RNA viral infection is pipendoxifene or a pharmaceutically acceptable salt, ester, solvate, stereoisomer, tautomer, or prodrug thereof.

MDL-001 (Pipendoxifene) has a chemical structure of:

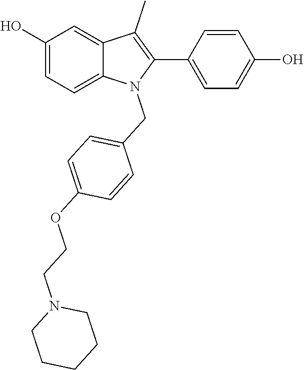 [SMILES: Cc1c(-c2ccc(O)cc2)n(Cc2ccc(OCCN3CCCCC3)cc2)c2ccc(O)cc12]

The chemical compounds disclosed herein can be RNA-dependent RNA polymerase (RdRp) inhibitors having high binding ability towards RdRp and specifically its Thumb-1 site, an allosteric subdomain of RdRp. As disclosed herein, these compounds can be used alone, or in combination with other therapeutic agent(s) for preventing, delaying the onset of, or treating an infection or disease caused by a RNA virus (e.g., HCV or coronavirus), and/or for preventing, delaying the onset of, or treating an inflammatory effect of an infection or a disease caused by a RNA virus (e.g., HCV or coronavirus).

RdRp is a polymerase that catalyzes the replication of RNA from an RNA template and is a vital enzyme for RNA viruses' relication/transcription complex. This enzyme synthesizes a full-length negative-strand RNA template that can subsequently be used to replicate and transcribe the viral genome. RdRp is an essential protein encoded in the genomes of most RNA-containing viruses that lack a DNA stage. RdRp's crucial role in the life cycle of RNA viruses has led to its targeted inhibition for a number of viral infections such as hepatitis C virus, Zika virus, and coronaviruses. The core RdRp domain consists of the thumb, palm and the fingers subdomains that are primarily involved in template binding, polymerization, nucleoside triphosphate (NTP) entry and associated functions. The palm subdomain is at the junction of the fingers and the thumb subdomains and houses most of the structurally conserved elements involved in catalysis. The catalytic aspartates and the RNA Recognizing Motif (RRM) comprising three β-strands are present in the palm subdomain. The thumb subdomain harbors residues that are involved in packing against the template RNA and stabilizing the initiating NTPs on the template. This subdomain also facilitates the translocation of template following polymerization by accommodating large conformational rearrangements.

The HCV RdRp, also known as NS5B, features a conserved hand-like structure with three domains—palm, fingers, and thumb—and harbors five identified allosteric sites (Thumb-1, Thumb-2, Palm-1, Palm-2, and Palm-3) for drug discovery efforts. Multiple sequence alignment of the Thumb-1 pocket of 35 positive-strand RNA viruses reveals a remarkable degree of conservation among the Thumb-1 pocket sequences. Multiple antivirals have been approved that target NS5B, including Beclabuvir (Thumb-1), Dasabuvir (Palm-1), and Sofosbuvir (active site). RdRp's crucial role in the life cycle of RNA viruses has led to its targeted inhibition for a number of viral infections such as hepatitis C virus, Zika virus, and coronaviruses. Current experimental drugs for this target include remdesivir, galidesivir, sofosbuvir, ribavirin, and favipiravir. Remdesivir is an antiviral inhibitor of RdRp that has shown to be effective against RNA viruses such as SARS-CoV, MERS-CoV and Ebola virus. The antiviral activity of Remdesivir is proposed due to its resemblance to an ATP used by RdRp. Remdesivir may be adequate to bind to the polymerase and hinder the enzyme's ability to incorporate additional RNA subunits, resulting in a failed genome replication. A recently discovered potent inhibitor known as ID-184 has been shown to bind RdRp more tightly than other experimental inhibitors.

The compounds disclosed herein (e.g., the compound provided in Table 1) can exhibit broad-spectrum antiviral activity across viral families through its inhibitory effect against RdRp and particularly its allosteric subdomain Thumb-1. As shown in the Examples, the compounds disclosed herein demonstrate enhanced target specificity with in vitro activity against HCV and human coronavirus, achieving a 100% hit rate. The in vitro studies conducted herein reveal that these compounds are highly potent and biologically relevant drug candidates with eliminated or minimal off-target activity. It is contemplated herein that the compounds from Table 1 have similar chemical and biological properties, and can bind to the conserved RdRp domain (e.g., Thumb-1 site) with enhanced binding ability and specificity as demonstrated herein, and thus can be used to treat broad-spectrum RNA viral infections. The compounds disclosed herein, in some embodiments, have low or no binding affinity to estrogen receptor. For example, the growth inhibition IC50 of the compound to estrogen receptor can be greater than 0.1 μM, 0.5 μM, 1 μM, 3 μM, 5 μM, 10 μM, 12 μM, 13 μM, 14 μM, 15 μM, 16 μM, 17 μM, 18 μM, 19 μM, 20 μM, 50 μM, or a value or a range between any two of these values.

In some embodiments, the compounds disclosed herein are designed to exclude undesired properties, specifically estrogen receptor interaction. The compounds disclosed herein demonstrate low estrogen receptor binding probability and negligible inhibition of estrogen receptor-mediated growth. The examples presented herein evaluate the compounds of Table 1 for selective estrogen regulation modulation activity using a growth inhibition assay, and the results reveal a successful removal of estrogen receptor binding from these compounds. In some embodiments, the estrogen receptor binding probability of the compounds in Table 1 is reduced by at least, or at least about 50%, 75%, 100%, 150%, 200%, 250%, 500%, 1000%, or higher and overlapping ranges therein, compared to other antiviral compounds, such as pipendoxifene.

The compounds disclosed herein include the compounds from Table 1 and stereoisomers, diasteteromers, conformational isomers as well as the racemates and pro-drugs thereof. The compounds disclosed herein (e.g., compounds from Table 1 or a pharmaceutically acceptable salt, ester, solvate, stereoisomer, tautomer, or prodrug thereof) can be used, for example, prevent or treat RNA viral infections. The RNA virus can be any RNA virus encoding and RNA-dependent RNA polymerase (RdRp). For example, a compound disclosed herein (or a pharmaceutically acceptable salt, ester, solvate, stereoisomer, tautomer, or prodrug thereof) can be administered to a patient in need (for example, a patient suffering from, or at a risk of developing, one or more of the RNA viral infections disclosed herein) at a daily dosage in the range of about 0.01 to 9000 mg administered orally, for an average adult human. It is recognized by those of skill in the art that the exact dosage may be adjusted depending on the severity of symptoms, body weight of the individual and/or other clinical circumstances existing in a given individual. Moreover, it is also recognized that dosage may be adjusted when a compound from Table 1 (or a pharmaceutically acceptable salt, ester, solvate, stereoisomer, tautomer, or prodrug thereof) is used in combination with other pharmacologically active substances.

### Pharmaceutical Compositions and Methods for Treatment

The compound from Table 1 or a pharmaceutically acceptable salt, ester, solvate, stereoisomer, tautomer, or prodrug thereof can be used in the methods and compositions disclosed herein for the prevention and treatment of disorders related to RNA viral infections. The RNA virus can be any RNA virus encoding an RNA-dependent RNA polymerase (RdRp), including coronavirus and hepatitis virus infections.

To prepare the pharmaceutical compositions of the present disclosure, the compounds from Table 1 can be intimately admixed with a pharmaceutically acceptable vehicle carrier according to conventional pharmaceutical compounding techniques, which may take a wide variety of forms depending on the form of preparation desired for administration (e.g., oral, transdermal, transmucosal, buccal, sublingual, transdermal, inhalation, nasal, rectal, vaginal, parenteral). In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like. Because of their ease in administration, tablets and capsules represent an advantageous oral dosage unit form, in which case solid pharmaceutical carriers are obviously employed. If desired, tablets may be sugar coated or enteric coated by standard techniques.

In addition, various controlled-release delivery methods, well known to those skilled in the art may be employed to improve bioavailability, reduce side effects, or transdermal delivery may be facilitated by various permeability enhancers or devises. Suppositories may be prepared, in which case cocoa butter could be used as the carrier. For parenterals, the carrier usually comprise sterile water, though other ingredients, for example, for purposes such as aiding solubility or for preservation, may be included. Injectable suspensions may also be prepared in which case appropriate liquid carriers, suspending agents and the like may be employed. Inhalable formulations and aerosols, topical formulations, nanoparticle and microparticle formulations and bioerodible and non-bioerodible formulations may also be prepared.

Included within the scope of the present disclosure are the various individual anomers, diastereomers and enantiomers as well as mixtures thereof, of a compound from Table 1. For example, the selective use of a particular enantiomer (e.g. R or S) of compounds from Table 1 to achieve a desired therapeutic effect is contemplated within the scope of the present disclosure since various enantiomers may have differential affinities for the receptor related to RNA viral infection. Also contemplated herein is the selective combination of various individual isomers, such as enantiomers in specific ratios (e.g. 3R:1S) to achieve a therapeutic effect. In addition, the compounds disclosed herein also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates and other solvates of the compound of Table 1 are included within the scope of the present disclosure.

Pharmaceutically acceptable salts of the compounds from Table 1 can be prepared by reacting the derivatives from Table 1 with the appropriate base and recovering the salt. In some embodiments, a compound from Table 1 is administered to the subject in a dosage of about 5-25 mg twice daily, or about 50 mg two or three times daily, or 100 mg once, twice or three times daily.

Also included within the scope of the present disclosure are various pro-drugs that may be converted by various physiologic processes into the active drug substance or which otherwise improves the bioavailability and/or pharmacological characteristics of the compounds disclosed herein. It is known to those of skill in the art that such pro-dugs may be created by creating derivatives of the compound from Table 1 which may be changed by normal physiologic and/or metabolic processes occurring with the individual into the pharmacologically active molecules from Table 1 or by combining the compound from Table 1 with another molecule or promoiety so as to enhance or control for example; absorption, distribution, metabolism and/or excretion in an individual.

The present disclosure also encompasses prodrugs of the compounds disclosed herein, which on administration undergo chemical conversion by metabolic processes before becoming active pharmacological substances. In general, such prodrugs are functional derivatives of the present compounds, which are readily convertible in vivo into the required compound from Table 1. Prodrugs are any covalently bonded compounds, which release the active parent drug from Table 1 in vivo. In cases in which compounds have unsaturated carbon-carbon double bonds, both the cis (Z) and trans (E) isomers are within the scope of the present disclosure. In cases wherein compounds may exist in tautomeric forms, such as ketoenol tautomers, each tautomeric form is contemplated as being included within the present disclosure whether existing in equilibrium or predominantly in one form. Conventional procedures for the selection and preparation of suitable prodrug derivatives are described, for example, in “Design of Prodrugs”, ed. H. Bundgaard, Elsevier, 1985. Prodrug designs are generally discussed in Hardma et al. (eds.), Goodman and Gilman's The Pharmacological Basis of Therapeutics, 9th ed., pages 11-16 (1996). A further thorough study of prodrug design is presented in Higuchi et al., Prodrugs as Novel Delivery Systems, vol. 14, ASCD Symposium Series, and in Roche (ed.), Bioreversible Carriers in Drug Design, American Pharmaceutical Association and Pergamon Press (1987).

The compounds from Table 1 can be linked, coupled or otherwise attached to another molecule which would facilitate the transport of the compounds or derivatives across cellular or tissue barriers. For example, gastrointestinal absorption can be enhanced by coupling, linking or attaching to another molecule such as a bile acid derivative or analogues to exploit the intestinal bile acid uptake pathway so as to enhance the intestinal absorption. Examples of such conjugations of a specific drug molecule with a carrier molecule, for example a bile acid, are well known to those familiar with the art. For example, Kramer (Biochim. Biophys. Acta. 1227: 137-154, 1994b) describes the conjugation of bile acids with cholesterol lowering drugs (i.e. HMG-CoA reductase inhibitors) for example lovastatin to improve gastrointestinal absorption and to facilitate more specific target organ drug delivery.

In addition, the compounds from Table 1 can be linked, coupled or otherwise attached to molecules which improve penetration of the blood brain barrier. For example, coupling, linking or attaching the compounds or derivatives to an essential fatty acid or vitamin to improve penetration into the central nervous system. Such techniques and a large range of molecules and promoieties which can achieve these effects are well known to those skilled in the art of pharmaceutical science. Methods to produce prodrugs using choline derivatives are described in US Patent Application published as US2001007865. The specific examples noted in the foregoing examples are provided for illustrative purposes and are not meant in any way to limit the scope contemplated herein.

The compounds contemplated in the scope of the present disclosure may be used in conjunction with one or more other therapeutic agents (e.g., drug compounds) and used according to the methods of the present disclosure, for example the therapeutic agents have a use that is also effective in treating RNA viral infection and/or co-morbid conditions.

When administered, the pharmaceutical composition comprising one or more of the compounds disclosed herein are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded herein. Such pharmacologically and pharmaceutically acceptable salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts. Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The compound from Table 1 are preferred to be administered in safe and effective amounts. An effective amount means that amount necessary to delay; the onset, inhibit the progression, halt altogether the onset or progression of, or to reduce the clinical manifestations or symptoms of the particular condition being treated. In general, an effective amount for treating an RNA viral infection are an amount necessary to inhibit the symptoms of the particular RNA viral infection in situ in a particular individual. When administered to an individual, effective amounts depends on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a minimum dose be used, that is, the lowest safe dosage that provides appropriate relief of symptoms.

Dosage can be adjusted appropriately to achieve desired drug levels, locally or systemically. Daily doses of active compounds can be from about 0.001 mg/kg per day to 200 mg/kg per day. However, it is recognized that these are general ranges and the actual dose used as contemplated in a given individual may less or greater than this dosage range. In the event that the response in an individual subject is insufficient at such doses, even higher doses (or effective higher doses by a different, more localized delivery route) may be employed to the extent that patient tolerance permits.

A variety of administration routes can be suitable to the methods and compositions disclosed herein. The particular administration route selected can depend upon the particular drug selected, the severity of the disease state(s) being treated and the dosage required for therapeutic efficacy. The methods may be practiced using any mode of administration that is medically acceptable, meaning any mode that produces effective levels of the active compounds without causing clinically unacceptable adverse effects and multiple doses over a given period of time are also contemplated. Such modes of administration include oral, rectal, sublingual, transmucosal, buccal, inhalation, rectal, vaginal, parenteral topical, nasal, transdermal or parenteral routes. The term “parenteral” includes subcutaneous, intravenous, intramuscular, or infusion. Depot intramuscular injections suitably prepared may also be used for administration within the scope of the present disclosure.

The compositions may be conveniently presented in unit dosage form and may be prepared by any of the methods well known in the art of pharmacy. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as; a syrup, an elixir, or an emulsion.

Other delivery systems can include time-release, delayed release, sustained release or targeted release delivery systems. Such systems can avoid repeated administrations of the active compounds, increasing convenience to the subject and the physician or target release of the active compound to the tissue of interest. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation, others of which are adapted for inhalation administration by nose or mouth.

Long-term sustained release devices, pharmaceutical compositions or molecular derivatives also may be used with the compounds described herein. “Long-term” release, as used herein, means that the drug delivery devise is constructed and arranged to deliver therapeutic levels of the active ingredient for at least 2 days, and preferably as long as 60 days. Long-term sustained release devices such as patches, implants and suppositories are well known to those of ordinary skill in the art and include some of the release systems described above. It is also contemplated by the inventors that the compounds described by the inventors may be formulated in such ways as to achieve various plasma profiles of the compounds in given individuals so as to maintain certain effective profiles of given plasma levels over a period of time. Such formulation strategies are well known to those skilled in the art and may for example include special coatings on tablets or granules containing the compounds disclosed herein either alone or in combination with other pharmacologically active substances. All such formulations are contemplated with the scope of the present disclosure.

### Methods for Treating Viral Infection

Disclosed herein include methods for preventing, delaying the onset of, or treating an infection or a disease caused by a RNA virus. In some embodiments, the method comprises administering to a subject in need thereof a composition comprising a compound disclosed herein (e.g., a compound selected from the compounds listed in Table 1) or a pharmaceutically acceptable salt, ester, solvate, stereoisomer, tautomer, or prodrug thereof, thereby preventing, delaying the onset of, or treating the infection or the disease. Disclosed herein include methods for preventing, delaying the onset of, or treating an inflammatory effect of an infection or a disease caused by a RNA virus. In some embodiments, the method comprises administering to a subject in need thereof a composition comprising a compound disclosed herein (e.g., a compound selected from the compounds listed in Table 1) or a pharmaceutically acceptable salt, ester, solvate, stereoisomer, tautomer, or prodrug thereof, thereby preventing, delaying the onset of, or treating the inflammatory effect.

Disclosed herein include methods for preventing, delaying the onset of, or treating an infection or a disease caused by a RNA virus. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease. Disclosed herein include methods for preventing, delaying the onset of, or treating an inflammatory effect of an infection or a disease caused by a RNA virus. In some embodiments, the method comprises: administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect.

The inflammatory effect can comprise respiratory failure, a sequela of respiratory failure, acute lung injury, or acute respiratory distress syndrome. The sequela of respiratory failure can comprise multi-organ failure. As used herein, the terms “inflammation” and “inflammatory response” shall be given their ordinary meaning, and also include immune-related responses and/or allergic reactions to a physical, chemical, or biological stimulus. Measuring inflammation (e.g. lung or liver inflammation) can comprise measuring the level of a pro-inflammatory cytokine, an anti-inflammatory cytokine, or a combination of pro-inflammatory cytokines and anti-inflammatory cytokines. Inflammation (e.g. lung inflammation) can comprise mast cell degranulation, plasma extravasation, and bronchoconstriction. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of one or more of mast cell degranulation, plasma extravasation, and bronchoconstriction. In some embodiments of the methods and compositions provided herein, lymphopenia and/or mononuclear cell infiltration in the lungs is reduced by at least, or at least about, 2% (e.g., 2%, 3%, 4%, 5%, 6%, 7%, 8%, 9%, 10%, 11%, 12%, 13%, %14%, 15%, 16%, 17%, 18%, 19%, 20%, 25%, 30%, 40%, 50%, 75%, 100%, 150%, 200%, 250%, 500%, 1000%, or higher and overlapping ranges therein).

A pro-inflammatory cytokine or a pro-inflammatory mediator can be an immuno-regulatory cytokine that favor inflammation. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-8, IL-11, IL-12, IL-18, GM-CSF, IFN-γ, TGF-β, leukemia inhibitory factors (LIF), oncostatin M (OSM), and a variety of chemokines that attract inflammatory cells. A pro-inflammatory cytokine generally up-regulates or increases the synthesis of secondary pro-inflammatory mediators and other pro-inflammatory cytokines by immune cells. In addition, pro-inflammatory cytokines can stimulate production of acute phase proteins that mediate inflammation and attract inflammatory cells. The method can comprise an at least, or at least about, 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) reduction in the level of one or more of interferon-γ (IFNγ), IL-1, IL-6, transforming growth factor-α (TGFα), transforming growth factor-β (TGFβ), CCL2, CXCL10, IL-11, IL-12, IL-18, GM-CSF, CXCL9 and IL-8 in the subject. The compositions and methods provided herein can reduce the production and/or amount of a pro-inflammatory cytokine and/or a pro-inflammatory mediator in the lung and/or serum by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used. In some embodiments, the inflammatory effect can comprise acute hepatitis, chronic hepatitis, liver swelling, liver damage, joint inflammation, muscle pain and weakness, or blood vessel problems.

The composition can comprise a therapeutically or prophylactically effective amount of one or more compounds disclosed herein. The subject in need can be a subject that can be suffering from the infection or the disease, or a subject that can be at a risk for the infection or the disease. The infection or the disease can be in the respiratory tract of the subject. The subject can have been exposed to the RNA virus, can be suspected to have been exposed to the RNA virus, or can be at a risk of being exposed to the RNA virus. The subject can be a mammal. The subject can be a human.

The RNA virus can be any RNA virus encoding an RNA-dependent RNA polymerase (RdRp). In some embodiments, the RNA virus can be a double-stranded RNA virus. The RNA virus can be a positive-sense single-stranded RNA virus. The positive-sense single-stranded RNA virus can be a coronavirus. The coronavirus can be an alpha coronavirus, a beta coronavirus, a gamma coronavirus, or a delta coronavirus. The coronavirus can be human coronavirus 229E (HCoV-229E), human coronavirus NL63 (HCoV-NL63), human coronavirus OC43 (HCoV—OC43), human coronavirus HKU1 (HCoV—HKU1), Middle East respiratory coronavirus (MERS-CoV), severe acute respiratory syndrome coronavirus (SARS-CoV), or SARS-CoV-2. In some embodiments, the positive-sense single-stranded RNA virus is hepatitis C virus. The infection or a disease caused by the RNA virus can be common cold, influenza, SARS, coronaviruses, COVID-19, hepatitis C, hepatitis E, West Nile fever, Ebola virus disease, rabies, polio, or measles.

The method can comprise administering to the subject one or more additional antiviral agents. At least one of the one or more additional antiviral agents can be co-administered to the subject with the composition. At least one of the one or more additional antiviral agents can be administered to the subject before the administration of the composition, after the administration of the composition, or both. The composition can comprise one or more additional therapeutic agents. The one or more additional therapeutic agents comprise one or more antiviral agents. The antiviral agent can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

The composition can be a pharmaceutical composition comprising a disclosed compound and one or more pharmaceutically acceptable excipients. The composition can be administered to the subject by intravenous administration, nasal administration, pulmonary administration, oral administration, parenteral administration, or nebulization. The composition can be aspirated into at least one lung of the subject. The composition can be in the form of powder, pill, tablet, microtablet, pellet, micropellet, capsule (including capsule containing microtablets), liquid, aerosols, or nanoparticles. The composition can be in a formulation for intravenous administration. The composition can be in a formulation for administration to the lungs. The compounds of the disclosure (e.g. a compound listed in Table 1, or a pharmaceutically acceptable salt, ester, solvate, stereoisomer, tautomer, or prodrug thereof) can also be used prophylactically for preventing, delaying the onset of, or treating an infection or a disease or inflammation caused by a RNA virus. The prophylactically effective amount of a compound of the disclosure can be any therapeutically effective amount of a compound described herein.

The compounds of the disclosure (e.g. a compound listed in Table 1, or a pharmaceutically acceptable salt, ester, solvate, stereoisomer, tautomer, or prodrug thereof) can be administered via any suitable route. Potential routes of administration of a disclosed compound include without limitation oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal and vaginal). In certain embodiments, a disclosed compound is administered orally (e.g., as a capsule or tablet, optionally with an enteric coating). In other embodiments, a disclosed compound is administered parenterally (e.g., intravenously, subcutaneously or intradermally). In further embodiments, a disclosed compound is administered topically (e.g., dermally/epicutaneously, transdermally, mucosally, transmucosally, buccally or sublingually).

In some embodiments, a disclosed compound is administered without food. In some embodiments, a disclosed compound is administered at least about 1 or 2 hours before or after a meal. In certain embodiments, a disclosed compound is administered at least about 2 hours after an evening meal. The compound disclosed herein can also be taken substantially concurrently with food (e.g., within about 0.5, 1 or 2 hours before or after a meal, or with a meal).

The composition can be administered to the subject once, twice, or three times a day. The composition can be administered to the subject once every day, every two days, or every three days. The composition can be administered to the subject over the course of at least two weeks, at least three weeks, at least four weeks, or at least five weeks. The therapeutically effective amount and the frequency of administration of, and the length of treatment with, a disclosed compound may depend on various factors, including the nature and the severity of the lung inflammation and/or infection/disease, the potency of the compound, the mode of administration, the age, the body weight, the general health, the gender and the diet of the subject, and the response of the subject to the treatment, and can be determined by the treating physician. In some embodiments, a disclosed compound (e.g., a compound listed in Table 1, or a pharmaceutically acceptable salt, ester, solvate, stereoisomer, tautomer, or prodrug thereof) is administered under a chronic dosing regimen. In certain embodiments, a therapeutically effective amount of a disclosed compound is administered over a period of at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).

A disclosed compound can also be used prophylactically to for preventing, delaying the onset of, or treating an infection or a disease or inflammation caused by a RNA virus. The prophylactically effective amount of a disclosed compound can be any therapeutically effective amount of a compound described herein.

Administrating the composition can result in reduction of the viral titer of the RNA virus in the subject as compared to that in the subject before administration of the composition. The method can comprise determining global virus distribution in the lungs of the subject. The method can comprise measuring the viral titer of the RNA virus in the subject before administering the composition to the subject, after administering the composition to the subject, or both. The viral titer can be lung bulk virus titer.

The method can comprise measuring a neutrophil density within the lungs of the subject. Administering the composition can result in reduction of the neutrophil density within the lungs of the subject as compared to that in the subject before administration of the composition. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the neutrophil density within the lungs of the subject as compared to that in the subject before administration of the composition.

The method can comprise measuring a total necrotized cell count within the lungs of the subject. Administering the composition can result in reduction of the total necrotized cell count in the subject as compared to that in the subject before administration of the composition. The method can comprise measuring a total protein level within the lungs of the subject. Administering the composition can result in reduction of the total protein level within the lungs of the subject as compared to that in the subject before administration of the composition. In some embodiments, administering the composition results in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the total protein level within the lungs of the subject as compared to that in the subject before administration of the composition.

In some embodiments, the method can comprise measuring liver enzyme levels (e.g., alanine aminotransferase (ALT) and aspartate aminotransferase (AST)) in the blood of the subject. In some embodiments, the method can comprise measuring liver enzymes prior to and after the administration of the composition. The compositions and methods provided herein can reduce the liver enzyme level by at least, or at least about 2% (e.g., 2%, 3%, 4%, 5%, 6%, 7%, 8%, 9%, 10%, 11%, 12%, 13%, 14%1, 15%, 16%, 17%, 18%, %19%, 20%, 25%, 30%, 40%, 50%, 75%, 100%, 150%, 200%, 250%, 500%, 1000%, or higher and overlapping ranges therein) compared to if the methods and compositions are not used.

In some embodiments, the method can comprise measuring albumin level in the blood of the subject. In some embodiments, the method can comprise measuring the albumin level prior to and after the administration of the composition. The compositions and methods provided herein can increase the albumin level by at least, or at least about 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

In some embodiments, the method can comprise measuring HCV RNA and/or anti-HCV antibody level in the subject. In some embodiments, the method can comprise measuring the HCV RNA and/or anti-HCV antibody level prior to and after the administration of the composition. The compositions and methods provided herein can reduce the HCV RNA and/or anti-HCV antibody level by at least, or at least about 2% (e.g., 2%, 3%, 4%, 5%, 6%, 7%, 8%, 9%, 10%, 11%, 12%, 13%, 14%, 15%, 16%, 17%, 18%, 1⁹%, 20%, 25%, 30%, 40%, 50%, 75%, 100%, 150%, 200%, 250%, 500%, 1000%, or higher and overlapping ranges therein) compared to if the methods and compositions are not used.

### Combination Therapy

Disclosed herein include methods for preventing, delaying the onset of, or treating an infection or a disease caused by a RNA virus. In some embodiments, the method comprises administering to a subject in need thereof a compound disclosed herein (e.g., a first compound selected from the compounds listed in Table 1) or a pharmaceutically salt, ester, solvate, stereoisomer, tautomer, or prodrug thereof, and a second compound disclosed herein (e.g., a second compound selected from the compounds listed in Table 1) or a pharmaceutically salt, ester, solvate, stereoisomer, tautomer, or prodrug thereof, thereby preventing, delaying the onset of, or treating the inflammatory effect, wherein the first compound and the second compound are different.

Disclosed herein include methods for preventing, delaying the onset of, or treating an inflammatory effect of an infection or a disease caused by a RNA virus. In some embodiments, the method comprises administering to a subject in need thereof one or more of the compounds disclosed herein (e.g., a first compound selected from the compounds listed in Table 1) or a pharmaceutically salt, ester, solvate, stereoisomer, tautomer, or prodrug thereof, and one or more of the compounds disclosed herein (e.g., a second compound selected from the compounds listed in Table 1) or a pharmaceutically acceptable salt, ester, solvate, stereoisomer, tautomer, or prodrug thereof, thereby preventing, delaying the onset of, or treating the inflammatory effect, wherein the first compound and the second compound are different.

Disclosed herein include methods for preventing, delaying the onset of, or treating an infection or a disease caused by a RNA virus. In some embodiments, the method comprises administering to a subject in need thereof one or more of the compounds disclosed herein (e.g., a compound from Table 1) or a pharmaceutically salt, ester, solvate, stereoisomer tautomer or prodrug thereof, and pipendoxifene or a pharmaceutically salt, ester, solvate, stereoisomer, tautomer, or prodrug thereof, thereby preventing, delaying the onset of, or treating the inflammatory effect, wherein the first compound and the second compound are different.

Disclosed herein include methods for preventing, delaying the onset of, or treating an inflammatory effect of an infection or a disease caused by a RNA virus. In some embodiments, the method comprises administering to a subject in need thereof one or more of the compounds disclosed herein (e.g., a compound from Table 1) or a pharmaceutically salt, ester, solvate, stereoisomer tautomer or prodrug thereof, and pipendoxifene or a pharmaceutically acceptable salt, ester, solvate, stereoisomer, tautomer, or prodrug thereof, thereby preventing, delaying the onset of, or treating the inflammatory effect, wherein the first compound and the second compound are different.

As disclosed herein, co-administration of particular ratios and/or amounts of a disclosed compound and one or more additional therapeutic agents (e.g., antiviral agents) can result in synergistic effects with regards to preventing, delaying the onset of, or treating an infection or a disease or inflammation caused by a RNA virus. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be, be about, be greater than, or be greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is and that is reduced to a greater degree as compared to the sum of the effects of administering each component, determined as if each component exerted its effect independently, also referred to as the predicted additive effect herein. For example, if a composition comprising component (a) yields an effect of a 20% reduction in lung inflammation and a composition comprising component (b) yields an effect of 50% reduction in lung inflammation, then a composition comprising both component (a) and component (b) would have a synergistic effect if the combination composition's effect on lung inflammation was greater than 70%.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least, or at least about, 20, 50, 75, 90, 100, 150, 200 or 300% greater than the predicted additive effect. In some embodiments, the synergistic effect can be at least, or at least about, 0.2, 0.5, 0.9, 1.1, 1.5, 1.7, 2, or 3 fold greater than the predicted additive effect.

In some embodiments, the synergistic effect of the combination compositions can also allow for reduced dosing amounts, leading to reduced side effects to the subject and reduced cost of treatment. Furthermore, the synergistic effect can allow for results that are not achievable through any other treatments. Therefore, proper identification, specification, and use of combination compositions can allow for significant improvements. The inflammatory effect can comprise respiratory failure, a sequela of respiratory failure, acute lung injury, or acute respiratory distress syndrome. The sequela of respiratory failure can comprise multi-organ failure.

The method can comprise measuring the viral titer of the RNA virus in the subject before administering the first, second and/or the third compound to the subject, after administering the first, second and/or the third compound to the subject, or both. The viral titer can be lung bulk virus titer. Administrating the first, second and/or the third compound can result in reduction of the viral titer of the RNA virus in the subject as compared to that in the subject before administration of the first, second and/or the third compound. The method can comprise determining global virus distribution in the lungs of the subject.

The method can comprise measuring a neutrophil density within the lungs of the subject. Administering the first, second and/or the third compound can result in reduction of the neutrophil density within the lungs of the subject as compared to that in the subject before administration of the first, second and/or the third compound. The method can comprise measuring a total necrotized cell count within the lungs of the subject. Administering the first, second and/or the third compound can result in reduction of the total necrotized cell count in the subject as compared to that in the subject before administration of the first, second and/or the third compound.

The method can comprise measuring a total protein level within the lungs of the subject. Administering the first, second and/or the third compound can result in reduction of the total protein level within the lungs of the subject as compared to that in the subject before administration of the first, second and/or the third compound.

The method can comprise detecting antibodies against the virus such as detecting anti-HCV antibodies (e.g., enzyme immunoassay to detect HCV antibody) and conducting molecular tests to detect HCV RNA in the blood using, for example, polymerase chain reaction to detect the HCV virus and measure virus level. In some embodiments, the method can comprise identify and monitor live damage from HCV, including measuring albumin level, performing liver function tests, prothrombin time, liver biopsy etc.

In some embodiments, the method can comprise measuring liver enzymes such as alanine aminotransferase (ALT) and aspartate aminotransferase (AST). In some embodiments, the method can comprise measuring liver enzymes prior to and after the administration of the composition comprising of the first, second and/or the third compound. Administering the first, second and/or the third compound can result in reduction of the liver enzyme level within the blood of the subject as compared to that in the subject before administration of the first, second and/or the third compound.

In some embodiments, the method can comprise measuring albumin levels of a subject. In some embodiments, the method can comprise measuring the albumin level prior to and after the administration of the composition comprising of the first, second and/or the third compound. Administering the first, second and/or the third compound can result in elevation of the albumin level within the blood of the subject as compared to that in the subject before administration of the first, second and/or the third compound.

The method can comprise measuring HCV RNA and/or anti-HCV antibody in the blood of the subject. Administering the first, second and/or the third compound can result in reduction of the HCV RNA level and/or anti-HCV antibody within the blood of the subject as compared to that in the subject before administration of the first, second and/or the third compound.

### Kits and Compositions

Disclosed herein include kits comprising one or more of the compounds disclosed herein (e.g., a first compound selected from the compounds listed in Table 1) or a pharmaceutically acceptable salt, solvate, stereoisomer thereof; and a label indicating that the kit is for preventing, delaying the onset of, or treating an infection or a disease caused by a RNA virus.

Disclosed herein include kits comprising one or more of the compounds disclosed herein (e.g., a first compound selected from the compounds listed in Table 1) or a pharmaceutically acceptable salt, solvate, stereoisomer thereof; and a label indicating that the kit is for preventing, delaying the onset of, or treating an inflammatory effect of an infection or a disease caused by a RNA virus.

The kit can further comprise one or more of the compounds disclosed herein (e.g., a second compound selected from the compounds listed in Table 1) or a pharmaceutically acceptable salt, ester, solvate, stereoisomer, tautomer, or prodrug thereof, wherein the first compound and the second compound is different. In some embodiments, the second compound can be pipendoxifene or a pharmaceutically acceptable salt, ester, solvate, stereoisomer, tautomer, or prodrug thereof.

The kit can comprise one or more of the compounds disclosed herein (e.g., a third compound selected from the compounds listed in Table 1) or a pharmaceutically acceptable salt, solvate, stereoisomer, tautomer, or prodrug thereof, wherein the first, second and third compound are different. In some embodiments, the third compound can be pipendoxifene or a pharmaceutically acceptable salt, ester, solvate, stereoisomer, tautomer, or prodrug thereof.

The RNA virus can be a coronavirus. The coronavirus can be human coronavirus 229E (HCoV-229E), human coronavirus NL63 (HCoV-NL63), human coronavirus OC43 (HCoV—OC43), human coronavirus HKU1 (HCoV—HKU1), Middle East respiratory coronavirus (MERS-CoV), severe acute respiratory syndrome coronavirus (SARS-CoV), or SARS-CoV-2. The RNA virus can be a hepatitis C virus.

Disclosed herein include compositions comprising one or more of the compounds disclosed herein (e.g., a compound selected from the compounds listed in Table 1) or a pharmaceutically acceptable salt, ester, solvate, stereoisomer, tautomer, or prodrug thereof, for use in preventing, delaying the onset of, or treating an infection or a disease caused by a RNA virus.

Disclosed herein include compositions comprising one or more of the compounds disclosed herein (e.g., a compound selected from the compounds listed in Table 1) or a pharmaceutically acceptable salt, ester, solvate, stereoisomer, tautomer, or prodrug thereof, for use in preventing, delaying the onset of, or treating an inflammatory effect of an infection or a disease caused by a RNA virus.

The inflammatory effect can comprise respiratory failure, a sequela of respiratory failure, acute lung injury, or acute respiratory distress syndrome. The sequela of respiratory failure can comprise multi-organ failure. The inflammatory effect can comprise acute hepatitis, chronic hepatitis, liver swelling, liver damage, joint inflammation, muscle pain and weakness, or blood vessel problem. The composition can comprise a therapeutically or prophylactically effective amount of the compound.

The therapeutically effective amount and the frequency of administration of, and the length of treatment with, a disclosed compound (e.g., a compound listed in Table 1, or a pharmaceutically acceptable salt, ester, solvate, stereoisomer, tautomer, or prodrug thereof) may depend on various factors, including the nature and the severity of the inflammation and/or infection/disease, the potency of a disclosed compound (e.g., a compound listed in Table 1, or a pharmaceutically acceptable salt, ester, solvate, stereoisomer, tautomer, or prodrug thereof), the mode of administration, the age, the body weight, the general health, the gender and the diet of the subject, and the response of the subject to the treatment, and can be determined by the treating physician. In some embodiments, a therapeutically effective amount of a disclosed compound (e.g., a compound listed in Table 1, or a pharmaceutically acceptable salt, ester, solvate, stereoisomer, tautomer, or prodrug thereof) for treating or preventing inflammation, an infection, and/or a disease as described herein is about 0.1-200 mg, 0.1-150 mg, 0.1-100 mg, 0.1-50 mg, 0.1-30 mg, 0.5-20 mg, 0.5-10 mg or 1-10 mg (e.g., per day or per dose), or as deemed appropriate by the treating physician, which can be administered in a single dose or in divided doses. In certain embodiments, the therapeutically effective dose (e.g., per day or per dose) of a disclosed compound (e.g., a compound listed in Table 1, or a pharmaceutically acceptable salt, ester, solvate, stereoisomer, tautomer, or prodrug thereof) for treating or preventing inflammation, an infection, and/or a disease as described herein is about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), about 150-200 mg (e.g., about 150 mg, 175 mg or 200 mg), about 200-300 mg (e.g., about 200 mg, 220 mg, 240 mg, 260 mg, 280 mg, or 300 mg), about 300-400 mg (e.g., about 300 mg, 320 mg, 340 mg, 360 mg, 380 mg, or 400 mg), about 400-500 mg (e.g., about 400 mg, 420 mg, 440 mg, 460 mg, 480 mg, or 500 mg), about 500-600 mg (e.g., about 500 mg, 520 mg, 540 mg, 560 mg, 580 mg, or 600 mg), or about 600-700 mg (e.g., about 600 mg, 620 mg, 640 mg, 660 mg, 680 mg, or 700 mg). In some embodiments, one or more of the disclosed compounds (e.g., a compound listed in Table 1, or a pharmaceutically acceptable salt, ester, solvate, stereoisomer, tautomer, or prodrug thereof) is administered for treating or preventing inflammation, an infection, and/or a disease as described herein at a daily dose, weekly dose, and/or monthly dose of about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), about 150-200 mg (e.g., about 150 mg, 175 mg or 200 mg), about 200-300 mg (e.g., about 200 mg, 220 mg, 240 mg, 260 mg, 280 mg, or 300 mg), about 300-400 mg (e.g., about 300 mg, 320 mg, 340 mg, 360 mg, 380 mg, or 400 mg), about 400-500 mg (e.g., about 400 mg, 420 mg, 440 mg, 460 mg, 480 mg, or 500 mg), about 500-600 mg (e.g., about 500 mg, 520 mg, 540 mg, 560 mg, 580 mg, or 600 mg), or about 600-700 mg (e.g., about 600 mg, 620 mg, 640 mg, 660 mg, 680 mg, or 700 mg). The daily dose, weekly dose, and/or monthly dose of the disclosed compounds can comprise a single administration (e.g., a weekly dose can administered once per week) or multiple administrations. In some embodiments, the dosing regimen comprises administering one or more loading doses and one or more maintenance doses. The term “loading dose” shall be given its ordinary meaning, and shall also refer to a single dose or short duration regimen of a multiple doses having a dosage higher than one or more maintenance doses. A loading dose can, for example, rapidly increase the blood concentration level of disclosed compounds. In some embodiments, the loading dose can increase the blood concentration of a compound to a therapeutically effective level in conjunction with a maintenance dose of the compound. The loading dose can be administered once per day, or more than once per day (e.g., up to 4 times per day). The term “maintenance dose” as used herein shall be given its ordinary meaning, and shall also refer to a dose that is serially administered (e.g., at least twice) which is intended to either slowly raise blood concentration levels of a disclosed compound to a therapeutically effective level, or to maintain such a therapeutically effective level. The daily dose of the maintenance dose can lower than the total daily dose of the loading dose.

The RNA virus can be a double-stranded RNA virus. The RNA virus can be a positive-sense single-stranded RNA virus. The positive-sense single-stranded RNA virus can be a coronavirus. The coronavirus can be an alpha coronavirus, a beta coronavirus, a gamma coronavirus, or a delta coronavirus. The coronavirus can be human coronavirus 229E (HCoV-229E), human coronavirus NL63 (HCoV-NL63), human coronavirus OC43 (HCoV—OC43), human coronavirus HKU1 (HCoV—HKU1), Middle East respiratory coronavirus (MERS-CoV), severe acute respiratory syndrome coronavirus (SARS-CoV), or SARS-CoV-2. In some embodiments, the positive-sense single-stranded RNA virus is hepatitis C virus. The infection or a disease caused by the RNA virus can be common cold, influenza, SARS, coronaviruses, COVID-19, hepatitis C, hepatitis E, West Nile fever, Ebola virus disease, rabies, polio, or measles.

The composition can be a pharmaceutical composition comprising the compound and one or more pharmaceutically acceptable excipients. The composition can comprise one or more additional therapeutic agents. The one or more additional therapeutic agents comprise one or more antiviral agents. The one or more antiviral agents can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

The composition can be in the form of powder, pill, tablet, microtablet, pellet, micropellet, capsule, capsule containing microtablets, liquid, aerosols, or nanoparticles. The composition can be in a formulation for administration to the lungs. The composition can be in a formulation for intravenous administration. As disclosed herein, the compounds of the disclosure (e.g., a compound listed in Table 1, or a pharmaceutically acceptable salt, ester, solvate, stereoisomer, tautomer, or prodrug thereof) can be formulated for administration in a pharmaceutical composition comprising a physiologically acceptable surface active agents, carriers, diluents, excipients, smoothing agents, suspension agents, film forming substances, coating assistants, or a combination thereof. In some embodiments, a compound of the disclosure (e.g., a compound listed in Table 1, or a pharmaceutically acceptable salt, ester, solvate, stereoisomer, tautomer, or prodrug thereof) is formulated for administration with a pharmaceutically acceptable carrier or diluent. The compounds of the disclosure (e.g., a compound listed in Table 1, or a pharmaceutically acceptable salt, ester, solvate, stereoisomer, tautomer, or prodrug thereof) can be formulated as a medicament with a standard pharmaceutically acceptable carrier(s) and/or excipient(s) as is routine in the pharmaceutical art. The exact nature of the formulation will depend upon several factors including the desired route of administration. Compounds of the disclosure (e.g., a compound listed in Table 1, or a pharmaceutically acceptable salt, ester, solvate, stereoisomer, tautomer, or prodrug thereof) can be formulated for oral, intravenous, intragastric, intravascular or intraperitoneal administration.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose: starches, such as corn starch and potato starch; cellulose and its derivatives, such as sodium carboxymethyi cellulose, powdered tragacanth; malt; gelatin; talc; solid lubricants, such as stearic acid and magnesium stearate; calcium sulfate; vegetable oils, such as peanut oil, cottonseed oil, sesame oil, olive oil, com oil and oil of theobroraa; polyols such as propylene glycol, glycerine, sorbitol, mannitol, and polyethylene glycol; aiginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject therapeutic agent is basically determined by the way the composition is to be administered.

The compositions can be provided in unit dosage form. As used herein, a “unit dosage form” is a composition containing an amount of a compound of the disclosure (e.g., a compound listed in Table 1, or a pharmaceutically acceptable salt, ester, solvate, stereoisomer, tautomer, or prodrug thereof) that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan can recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compounds of the disclosure (e.g., a compound listed in Table 1, or a pharmaceutically acceptable salt, ester, solvate, stereoisomer, tautomer, or prodrug thereof). The amount of carrier employed in conjunction with the compounds of the disclosure (e.g., a compound listed in Table 1, or a pharmaceutically acceptable salt, ester, solvate, stereoisomer, tautomer, or prodrug thereof) is sufficient to provide a practical quantity of material for administration per unit dose of the disclosed compositions.

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polvsorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject therapeutic agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyi methyl cellulose. Glidants, lubricants, sweeteners, colorants, antioxidants and flavoring agents disclosed above may also be included.

For topical use, creams, ointments, gels, solutions or suspensions, etc., containing a compound of the disclosure (e.g., a compound listed in Table 1, or a pharmaceutically acceptable salt, ester, solvate, stereoisomer, tautomer, or prodrug thereof) disclosed herein are employed. Topical formulations may generally be comprised of a pharmaceutical carrier, co-solvent, emulsifier, penetration enhancer, preservative system, and emollient.

For intravenous administration, the compounds of the disclosure (e.g., a compound listed in Table 1, or a pharmaceutically acceptable salt, ester, solvate, stereoisomer, tautomer, or prodrug thereof) and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, suifoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Antimicrobial agents may also be included to achieve a bacteriostatic or fungistatic solution, including but not limited to phenyl mercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound of the disclosure (e.g., a compound listed in Table 1, or a pharmaceutically acceptable salt, ester, solvate, stereoisomer, tautomer, or prodrug thereof) and another agent, the combination may be provided to caregivers as a mixture, or the caregivers may mix the two agents prior to administration, or the two agents may be administered separately.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 0.1 mg/kg and 4000 mg/kg body weight, preferably between about 80 mg/kg and 1600 mg/kg body weight. Alternatively dosages may be based and calculated upon the surface area of the patient, as understood by those of skill in the art.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will depend on many factors including the subject being treated, the severity of the affliction, the manner of administration, the judgment of the prescribing physician. The compounds of the disclosure (e.g., a compound listed in Table 1, or a pharmaceutically acceptable salt, ester, solvate, stereoisomer, tautomer, or prodrug thereof) or combination of therapeutic agents disclosed herein may be administered orally or via injection at a dose from 0, 1 mg/kg to 4000 mg/kg of the patient's body weight per day. The dose range for adult humans is generally from 1 g to 100 g/day. Tablets or other forms of presentation provided in discrete units may conveniently contain an amount of a compound of the disclosure (e.g., a compound listed in Table 1, or a pharmaceutically acceptable salt, ester, solvate, stereoisomer, tautomer, or prodrug thereof) or combination of compounds disclosed herein which is effective at such dosage or as a multiple of the same, for instance, units containing 1 g to 60 g (for example, from about 5 g to 20 g, from about 10 g to 50 g, from about 20 g to 40 g, or from about 25 g to 35 g). The precise amount of therapeutic agent administered to a patient is the responsibility of the attendant physician. However, the dose employed can depend on a number of factors, including the age and sex of the patient, the precise disorder being treated, and its severity. Additionally, the route of administration may vary depending on the condition and its severity. A typical dose of the compounds of the disclosure (e.g., a compound listed in Table 1, or a pharmaceutically acceptable salt, ester, solvate, stereoisomer, tautomer, or prodrug thereof) can be from 0,02 g to 1.25 g per kg of body weight, for example from 0.1 g to 0.5 g per kg of body weight, depending on such parameters. In some embodiments, the dosage of a disclosed compound (e.g., a compound listed in Table 1, or a pharmaceutically acceptable salt, ester, solvate, stereoisomer, tautomer, or prodrug thereof) can be from 1 g to 100 g, for example, from 10 g to 80 g, from 15 g to 60 g, from 20 g to 40 g, or from 25 g to 35 g. In A physician will be able to determine the required dosage of the compounds of the disclosure (e.g., a compound listed in Table 1, or a pharmaceutically acceptable salt, ester, solvate, stereoisomer, tautomer, or prodrug thereof) for any particular subject.

The exact formulation, route of administration and dosage for the pharmaceutical compositions comprising one or more compounds of the disclosure (e.g., a compound listed in Table 1, or a pharmaceutically acceptable salt, ester, solvate, stereoisomer, tautomer, or prodrug thereof) or combination of therapeutic agents disclosed herein can be chosen by the individual physician in view of the patient's condition. (See, e.g., Fingl et al. 1975, in “The Pharmacological Basis of Therapeutics,” which is hereby incorporated herein by reference, with particular reference to Ch. 1). Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for therapeutic agents have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In cases of administration of a pharmaceutically acceptable salt, dosages may be calculated as the free base. In some embodiments, the composition is administered 1 to 4 times per day. Alternatively the compositions disclosed herein may be administered by continuous intravenous infusion, e.g., at a dose of each active ingredient up to 100 g per day. As will be understood by those of skill in the art, in certain situations it may be necessary to administer the compositions disclosed herein in amounts that exceed, or even far exceed, the above-stated, preferred dosage range in order to effectively and aggressively treat particularly aggressive diseases or infections. In some embodiments, a compound of the disclosure (e.g., a compound listed in Table 1, or a pharmaceutically acceptable salt, ester, solvate, stereoisomer, tautomer, or prodrug thereof) or combination of therapeutic agents disclosed herein will be administered for a period of continuous therapy, for example for a week or more, or for months or years.

In some embodiments, the dosing regimen of the compounds of the disclosure (e.g., a compound listed in Table 1, or a pharmaceutically acceptable salt, ester, solvate, stereoisomer, tautomer, or prodrug thereof) or combination of therapeutic agents disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of a compound of the disclosure (e.g., a compound listed in Table 1, or a pharmaceutically acceptable salt, ester, solvate, stereoisomer, tautomer, or prodrug thereof) or combination of therapeutic agents disclosed herein can be administered three times a day, twice a day, daily, every other day, three times a week, every other week, three times per month, once monthly, substantially continuously or continuously.

A compound disclosed herein can be administered alone or in the form of a composition (e.g., a pharmaceutical composition). In some embodiments, a pharmaceutical composition comprises one or more compounds of the disclosure (e.g., a compound listed in Table 1, or a pharmaceutically acceptable salt, ester, solvate, stereoisomer, tautomer, or prodrug thereof) or a pharmaceutically acceptable salt, solvate, hydrate, clathrate, polymorph, tautomer, prodrug or metabolite thereof, and one or more pharmaceutically acceptable carriers or excipients. The composition can optionally contain one or more additional therapeutic agents as described herein. A pharmaceutical composition contains a therapeutically effective amount of a compound of the disclosure (e.g., a compound listed in Table 1, or a pharmaceutically acceptable salt, ester, solvate, stereoisomer, tautomer, or prodrug thereof) and one or more pharmaceutically acceptable carriers or excipients, and is formulated for administration to a subject for therapeutic use. For purposes of the content of a pharmaceutical composition, the terms “therapeutic agent”, “active ingredient”, “active agent” and “drug” encompass prodrugs.

A pharmaceutical composition contains one or more compounds of the disclosure (e.g., a compound listed in Table 1, or a pharmaceutically acceptable salt, ester, solvate, stereoisomer, tautomer, or prodrug thereof) in substantially pure form. In some embodiments, the purity of the therapeutic agent is at least about 95%, 96%, 97%, 98% or 99%. In certain embodiments, the purity of the therapeutic agent is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline [PBS] and isotonic solutions [e.g., Ringer's solution]), and solvents (e.g., dimethyl sulfoxide [DMSO] and alcohols [e.g., ethanol, glycerol and propylene glycol]). Except insofar as any conventional carrier or excipient is incompatible with the active ingredient, the disclosure encompasses the use of conventional carriers and excipients in formulations containing a therapeutic agent (e.g., a compound of the disclosure, such as Tocladesine, PRX-07034, AZD-5991, Berzosertib, Pipendoxifene, Bazedoxifene, R-428).

Proper formulation can depend on various factors, such as the mode of administration chosen. Potential modes of administration of pharmaceutical compositions comprising a compound of the disclosure (e.g., a compound listed in Table 1, or a pharmaceutically acceptable salt, ester, solvate, stereoisomer, tautomer, or prodrug thereof) include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], buccal, sublingual, rectal [e.g., by suppository], and vaginal [e.g., by suppository]).

As an example, formulations of a compound of the disclosure (e.g., a compound listed in Table 1, or a pharmaceutically acceptable salt, ester, solvate, stereoisomer, tautomer, or prodrug thereof) suitable for oral administration can be presented as, e.g., boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Tablets can contain a compound of the disclosure (e.g., a compound listed in Table 1, or a pharmaceutically acceptable salt, ester, solvate, stereoisomer, tautomer, or prodrug thereof) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises a compound of the disclosure (e.g., a compound listed in Table 1, or a pharmaceutically acceptable salt, ester, solvate, stereoisomer, tautomer, or prodrug thereof), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain a compound of the disclosure (e.g., a compound listed in Table 1, or a pharmaceutically acceptable salt, ester, solvate, stereoisomer, tautomer, or prodrug thereof) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, a compound of the disclosure (e.g., a compound listed in Table 1, or a pharmaceutically acceptable salt, ester, solvate, stereoisomer, tautomer, or prodrug thereof) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of a compound of the disclosure (e.g., a compound listed in Table 1, or a pharmaceutically acceptable salt, ester, solvate, stereoisomer, tautomer, or prodrug thereof) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, a compound of the disclosure (e.g., a compound listed in Table 1, or a pharmaceutically acceptable salt, ester, solvate, stereoisomer, tautomer, or prodrug thereof) is contained in an amphiphilic vehicle of a liquid or semi-solid formulation for oral administration which provides improved solubility, stability and bioavailability of the compound, as described in US 2010/0209496. The amphiphilic vehicle contains a solution, suspension, emulsion (e.g., oil-in-water emulsion) or semi-solid mixture of the compound admixed with liquid or/and semi-solid excipients which fills an encapsulated dosage form (e.g., a hard gelatin capsule or a soft gelatin capsule containing a plasticizer [e.g., glycerol or/and sorbitol]). In some embodiments, the amphiphilic vehicle comprises an amphiphilic agent selected from fatty acid esters of glycerol (glycerin), propylene glycol and sorbitol. In certain embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, IMWITOR® 624, IMWITOR® 742, IMWITOR® 988, CAPRYOL™ PGMC, CAPRYOL™ 90, LAUROGLYCOL™ 90, CAPTEX® 200, CRILL™ 1, CRILL™ 4, PECEOL® and MAIS INE™ 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination thereof. In additional embodiments, the amphiphilic vehicle contains on a weight basis about 0.1-5% of the compound, about 50-90% of the amphiphilic agent, about 5-40% of propylene glycol, about 5-20% of the propylene glycol-sparing agent, and about 0.01-0.5% of the antioxidant.

A compound of the disclosure (e.g., a compound listed in Table 1, or a pharmaceutically acceptable salt, ester, solvate, stereoisomer, tautomer, or prodrug thereof) can also be formulated for parenteral administration by injection or infusion to circumvent gastrointestinal absorption and first-pass metabolism. A representative parenteral route is intravenous.

Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain a compound of the disclosure (e.g., a compound listed in Table 1, or a pharmaceutically acceptable salt, ester, solvate, stereoisomer, tautomer, or prodrug thereof) along with excipients such as an antioxidant, a buffer, a bacteriostat and solutes that render the formulation isotonic with the blood of the subject. Aqueous or non-aqueous sterile suspensions can contain a compound of the disclosure (e.g., a compound listed in Table 1, or a pharmaceutically acceptable salt, ester, solvate, stereoisomer, tautomer, or prodrug thereof) along with excipients such as a suspending agent and a thickening agent, and optionally a stabilizer and an agent that increases the solubility of the compound to allow for the preparation of a more concentrated solution or suspension. As another example, a sterile aqueous solution for injection or infusion (e.g., subcutaneously or intravenously) can contain a compound of the disclosure (e.g., a compound listed in Table 1, or a pharmaceutically acceptable salt, ester, solvate, stereoisomer, tautomer, or prodrug thereof), NaCl, a buffering agent (e.g., sodium citrate), a preservative (e.g., meta-cresol), and optionally a base (e.g., NaOH) or/and an acid (e.g., HCl) to adjust pH.

For topical administration, a compound of the disclosure (e.g., a compound listed in Table 1, or a pharmaceutically acceptable salt, ester, solvate, stereoisomer, tautomer, or prodrug thereof) can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the compound for more rapid uptake of it into systemic circulation. In addition to a therapeutically effective amount of a disclosed compound (e.g., a compound listed in Table 1, or a pharmaceutically acceptable salt, ester, solvate, stereoisomer, tautomer, or prodrug thereof), the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, a compound of the disclosure (e.g., a compound listed in Table 1, or a pharmaceutically acceptable salt, ester, solvate, stereoisomer, tautomer, or prodrug thereof) can also be formulated for intranasal administration. The nasal mucosa provides a big surface area, a porous endothelium, a highly vascular subepithelial layer and a high absorption rate, and hence allows for high bioavailability. An intranasal solution or suspension formulation can comprise a compound of the disclosure (e.g., a compound listed in Table 1, or a pharmaceutically acceptable salt, ester, solvate, stereoisomer, tautomer, or prodrug thereof) along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. In certain embodiments, a nasal spray formulation comprises a compound of the disclosure (e.g., a compound listed in Table 1, or a pharmaceutically acceptable salt, ester, solvate, stereoisomer, tautomer, or prodrug thereof), microcrystalline cellulose, sodium carboxymethylcellulose, dextrose and water, and optionally an acid (e.g., HCl) to adjust pH. An intranasal solution or suspension formulation can be administered to the nasal cavity by any suitable means, including but not limited to a dropper, a pipette, or spray using, e.g., a metering atomizing spray pump.

An additional mode of topical administration is pulmonary, including by oral inhalation and nasal inhalation, which is described in detail below. Other suitable topical formulations and dosage forms include without limitation ointments, creams, gels, lotions, pastes and the like.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g., menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments, a compound of the disclosure (e.g., a compound listed in Table 1, or a pharmaceutically acceptable salt, ester, solvate, stereoisomer, tautomer, or prodrug thereof) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In certain embodiments, the sustained-release composition delivers the compound over a period of at least about 1 day, 2 days, 3 days, 1 week, 2 weeks, 3 weeks, 1 month, 2 months, 3 months or longer. In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium poly acrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

Pharmaceutical compositions comprising a compound of the disclosure (e.g., a compound listed in Table 1, or a pharmaceutically acceptable salt, ester, solvate, stereoisomer, tautomer, or prodrug thereof) can be formulated as, e.g., liposomes, micelles (e.g., those composed of biodegradable natural or/and synthetic polymers, such as lactosomes), microspheres, microparticles or nanoparticles, whether or not designed for sustained release. For example, liposomes can be used as sustained release pulmonary drug-delivery systems that deliver drugs to the alveolar surface for treatment of lung diseases and systemic diseases.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of a therapeutic agent (e.g., a compound listed in Table 1, or a pharmaceutically acceptable salt, ester, solvate, stereoisomer, tautomer, or prodrug thereof). Representative examples of a unit dosage form include a tablet, capsule or pill for oral administration, and powder in a vial or ampoule for oral or nasal inhalation.

Alternatively, a pharmaceutical composition can be presented as a kit, wherein the active ingredient, excipients and carriers (e.g., solvents) are provided in two or more separate containers (e.g., ampoules, vials, tubes, bottles or syringes) and need to be combined to form the composition to be administered. The kit can contain instructions for storing, preparing and administering the composition (e.g., a solution to be injected intravenously). A kit can contain all active and inactive ingredients in unit dosage form or the active ingredient and inactive ingredients in two or more separate containers, and can contain instructions for using the pharmaceutical composition. The kit can contain a compound of the disclosure (e.g., a compound listed in Table 1, or a pharmaceutically acceptable salt, ester, solvate, stereoisomer, tautomer, or prodrug thereof) or a pharmaceutically acceptable salt, solvate, hydrate, clathrate, polymorph, prodrug or metabolite thereof, and instructions for administering the compound. In certain embodiments, the compound is contained or incorporated in, or provided by, a device or system configured for pulmonary delivery of the compound by oral inhalation, such as a metered-dose inhaler, a dry powder inhaler or a nebulizer.

Pulmonary administration can be accomplished by, e.g., oral inhalation or nasal inhalation. Advantages of pulmonary drug delivery include, but are not limited to: 1) avoidance of first pass hepatic metabolism; 2) fast drug action; 3) large surface area of the alveolar region for absorption, high permeability of the lungs (thin air-blood barrier), and profuse vasculature of the airways; 4) smaller doses to achieve equivalent therapeutic effect compared to other oral routes; 5) local action within the respiratory tract; 6) reduced systemic side effects; and 7) reduced extracellular enzyme levels compared to the gastrointestinal tract due to the large alveolar surface area. An advantage of oral inhalation over nasal inhalation includes deeper penetration/deposition of the drug into the lungs. Pulmonary administration, whether by oral or nasal inhalation, can be a suitable route of administration for drugs that are intended to act locally in the lungs or/and systemically, for which the lungs serve as a portal to the systemic circulation.

Oral or nasal inhalation can be achieved by means of, e.g., a metered-dose inhaler (MDI), a nebulizer or a dry powder inhaler (DPI). For example, a compound of the disclosure (e.g., a compound listed in Table 1, or a pharmaceutically acceptable salt, ester, solvate, stereoisomer, tautomer, or prodrug thereof) can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer. For example, an MDI formulation can comprise a compound of the disclosure (e.g., a compound listed in Table 1, or a pharmaceutically acceptable salt, ester, solvate, stereoisomer, tautomer, or prodrug thereof), a propellant (e.g., an HFA such as 1,1,1,2-tetrafluoroethane), a surfactant (e.g., a fatty acid such as oleic acid), and a co-solvent (e.g., an alcohol such as ethanol). The MDI formulation can optionally contain a dissolved gas (e.g., CO₂). After device actuation, the bursting of CO₂ bubbles within the emitted aerosol droplets breaks up the droplets into smaller droplets, thereby increasing the respirable fraction of drug. As another example, a nebulizer formulation can comprise a compound of the disclosure (e.g., a compound listed in Table 1, or a pharmaceutically acceptable salt, ester, solvate, stereoisomer, tautomer, or prodrug thereof), a surfactant (e.g., a Tween® such as polysorbate 80), a chelator or preservative (e.g., edetate disodium), an isotonicity agent (e.g., sodium chloride), pH buffering agents (e.g., citric acid/sodium citrate), and water. The drug can be delivered by means of, e.g., a nebulizer or an MDI with or without a spacer, and the drug dose delivered can be controlled by a metering chamber (nebulizer) or a metering valve (MDI).

Metered-dose inhalers (also called pressurized metered-dose inhalers [pMDI]) are the most widely used inhalation devices. A metering valve delivers a precise amount of aerosol (e.g., about 20-100 μL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easibreathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer.

Nebulizers generate aerosol droplets of about 1-5 microns. They do not require user coordination between device actuation and inhalation, which can significantly affect the amount of drug deposited in the lungs. Compared to MDIs and DPIs, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™ NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, Micro Air® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse.

Respimat® Soft Mist™ inhaler combines advantages of an MDI and a nebulizer. It is a small, hand-held inhaler that does not need a power supply (like an MDI) and slowly aerosolizes a propellant-free drug solution as a soft mist (like a nebulizer), thereby reducing drug deposition in the oropharyngeal region and increasing drug deposition in the central and peripheral lung regions. The Soft Mist™ inhaler can create a large fraction of respirable droplets with slow velocity from a metered volume of drug solution. A drug delivered from the Soft Mist™ inhaler can potentially achieve the same therapeutic outcome at a significantly lower dose compared to delivery from an MDI.

For oral or nasal inhalation using a dry powder inhaler (DPI), a compound of the disclosure (e.g., a compound listed in Table 1, or a pharmaceutically acceptable salt, ester, solvate, stereoisomer, tautomer, or prodrug thereof) can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de-agglomerate drug particles, rapid inspiration is not recommended with an MDI or nebulizer, since it creates turbulent airflow and fast velocity which increase drug deposition by impaction in the upper airways.) Compared to an MDI, a DPI (including a passive, breath-activated DPI) can potentially deliver larger doses of drug, and larger-size drugs (e.g., macromolecules), to the lungs.

Dry powder inhalers can be classified by dose type into single-unit dose (including disposable and reusable) and multi-dose (including multi-dose reservoirs and multi-unit dose). In a single-unit dose DPI, the formulation can be a powder mix of a micronized drug powder and a carrier and can be supplied in individual capsules, which are inserted into the inhaler for a single dose and are removed and discarded after use. The capsule body containing the dose falls into the device, while the cap is retained in the entry port for subsequent disposal. As the user inhales, the portion of the capsule containing the drug experiences erratic motion in the airstream, causing dislodged particles to be entrained and subsequently inhaled. Particle de-aggregation is caused mainly by turbulence promoted by the grid upstream of the mouthpiece or nosepiece. Examples of single-unit dose DPIs include without limitation Aerolizer®, AIR®, Conix One® (foil seal), Diskhaler®, Diskus®, Handihaler®, Microhaler®, Rotahaler® and Turbo Spin®.

A multi-unit dose DPI uses factory-metered and -sealed doses packaged in a manner so that the device can hold multiple doses without the user having to reload. The packaging typically contains replaceable disks or cartridges, or strips of foil-polymer blister packaging that may or may not be reloadable. For example, individual doses can be packaged in blister packs on a disk cassette. Following piercing, inspiratory flow through the packaging depression containing the drug induces dispersion of the powder. The aerosol stream is mixed with a bypass flow entering through holes in the mouthpiece or nosepiece, which gives rise to turbulence and promotes particle de-agglomeration. Advantages of the prepackaging include protection from the environment until use and ensurance of adequate control of dose uniformity. Examples of multi-unit dose DPIs include without limitation Acu-Breath®, Bulkhaler®, Certihaler®, DirectHaler®, Diskhaler®, Diskus®, Dispohaler®, M®, MF-DPI®, Miat-Haler®, NEXT DPI®, Prohaler®, Swinhaler® and Technohaler®.

### RNA Viruses

Disclosed herein include methods for preventing, delaying the onset of, or treating an infection, disease, or inflammation caused by a RNA virus. The present disclosure contemplates treating a broad range of viral diseases, including infections of all types, locations, sizes, and characteristics. The RNA virus can be a double-stranded RNA virus. The RNA virus can be a positive-sense single-stranded RNA virus. In some embodiments, the positive-sense single-stranded RNA virus is a coronavirus. The coronavirus can be an alpha coronavirus, a beta coronavirus, a gamma coronavirus, or a delta coronavirus. The coronavirus can be human coronavirus 229E (HCoV-229E), human coronavirus NL63 (HCoV-NL63), human coronavirus OC43 (HCoV—OC43), human coronavirus HKU1 (HCoV—HKU1), Middle East respiratory coronavirus (MERS-CoV), severe acute respiratory syndrome coronavirus (SARS-CoV), or SARS-CoV-2. In some embodiments, the positive-sense single-stranded RNA virus is a hepatitis virus (e.g., a hepatitis A, C, D or E virus). In some embodiments, the RNA virus is a hepatitis C virus, including genotypes 1-6.

The infection or disease caused by the RNA virus can be common cold, influenza, SARS, coronaviruses, COVID-19, hepatitis C, hepatitis E, West Nile fever, Ebola virus disease, rabies, polio, or measles.

The methods and compositions disclosed herein are useful for preventing, delaying the onset of, or treating an infection, disease, or inflammation caused by a RNA virus. The subject can have been exposed to the RNA virus, can be suspected to have been exposed to the RNA virus, or can be at a risk of being exposed to the RNA virus. The compositions may be used as a prophylactic (to prevent the development of a viral infection) or may be used to treat existing viral infections.

The RNA virus can be any RNA virus encoding an RNA-dependent RNA polymerase (RdRp). The RNA virus can be an enveloped virus. The RNA virus can a retrovirus. The RNA virus can be a filovirus, arenavirus, bunyavirus, or a rhabdovirus. The RNA virus can be a hepadnavirus, coronavirus, or a flavivirus. The RNA virus can be Respiratory syncytial virus, Parainfluenza virus, Enterovirus 71, Hantavirus, SARS virus, SARS-associated coronavirus, severe acute respiratory syndrome coronavirus (SARS-CoV), or SARS-CoV-2, Sin Nombre virus, Respiratory reovirus. The present disclosure encompasses the treatment of infections with derivatives of any of the viruses disclosed herein. As disclosed herein, the term “derivative of a virus” can refer to a strain of virus that has mutated from an existing viral strain.

The RNA virus can comprise any serotype of human rhinovirus (HRV). HRV may include, without limitation, the species Rhinovirus A (including, but not limited to, serotypes HRV-A1, HRV-A2, HRV-A7, HRV-A8, HRV-A9, HRV-A10, HRV-A11, HRV-A12, HRV-A13, HRV-A15, HRV-A16, HRV-A18, HRV-A19, HRV-A20, HRV-A21, HRV-A22, HRV-A23, HRV-A24, HRV-A25, HRV-A28, HRV-A29, HRV-A30, HRV-A31, HRV-A32, HRV-A33, HRV-A34, HRV-A36, HRV-A38, HRV-A39, HRV-A40, HRV-A41, HRV-A43, HRV-A44, HRV-A45, HRV-A46, HRV-A47, HRV-A49, HRV-A50, HRV-A51, HRV-A53, HRV-A54, HRV-A55, HRV-A56, HRV-A57, HRV-A58, HRV-A59, HRV-A60, HRV-A61, HRV-A62, HRV-A63, HRV-A64, HRV-A65, HRV-A66, HRV-A67, HRV-A68, HRV-A71, HRV-A73, HRV-A74, HRV-A75, HRV-A76, HRV-A77, HRV-A78, HRV-A80, HRV-A81, HRV-A82, HRV-A85, HRV-A88, HRV-A89, HRV-A90, HRV-A94, HRV-A95, HRV-A96, HRV-A98, HRV-A100, HRV-A101, HRV-A102 and HRV-A103), Rhino virus B (including, but not limited to, the serotypes HRV-B3, HRV-B4, HRV-B5, HRV-B6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91, HRV-B92, HRV-B93, HRV-B97, and HRV-B99), and Rhinovirus C (including, but not limited to, serotypes HRV-C1, HRV-C2, HRV-C3, HRV-C4, HRV-C5, HRV-C6, HRV-C7, HRV-C8, HRV-C9, HRV-C10, HRV-C11, HRV-C12, HRV-C13, HRV-C14, HRV-C15, HRV-C16, HRV-C17, HRV-C18, HRV-C19, HRV-C20, HRV-C21, HRV-C22, HRV-C23, HRV-C24, HRV-C25, HRV-C26, HRV-C27, HRV-C28, HRV-C29, HRV-C30, HRV-C31, HRV-C32, HRV-C33, HRV-C34, HRV-C35, HRV-C36, HRV-C37, HRV-C38, HRV-C39, HRV-C40, HRV-C41, HRV-C42, HRV-C43, HRV-C44, HRV-C45, HRV-C46, HRV-C47, HRV-C48, HRV-C49, HRV-C50 and HRV-C51).

In some embodiments the RNA virus is an influenza A virus. Non-limiting examples of influenza A viruses include subtype H10N4, subtype H10N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of influenza A virus include, but are not limited to: A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/i/81 (H1N1); A/sw/Potsdam/i/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d′Armor/3633/84 (H3N2); A/sw/Gent/i/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/GentN230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/i/97 (H3N2); A/sw/Cote d′Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d′Armor/604/99 (H1N2); A/sw/Cote d′Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d′Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselunne/2617/03 hp (H1N1); A/sw/Loningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dotlingen/IDT3780/05 (H1N2); A/sw/Dotlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/swNoglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R_{169/2006} (H3N2).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1); A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

In some embodiments the RNA virus is an influenza B virus. Non-limiting examples of influenza B viruses include strain Aichi/5/88, strain Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos AiresNL518/99, strain Canada/464/2001, strain Canada/464/2002, strain Chaco/366/00, strain Chaco/R_{113/00}, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA110 Philippines/2002), strain clinical isolate SA112 Philippines/2002, strain clinical isolate SA113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain CordobaNA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/i/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, and strain Rochester/02/2001.

In some embodiments, the RNA virus is an influenza C virus. Non-limiting examples of influenza C viruses include strain Aichi/1/81, strain Ann Arbor/i/50, strain Aomori/74, strain California/78, strain England/83, strain Greece/79, strain Hiroshima/246/2000, strain Hiroshima/252/2000, strain Hyogo/1/83, strain Johannesburg/66, strain Kanagawa/1/76, strain Kyoto/1/79, strain Mississippi/80, strain Miyagi/1/97, strain Miyagi/5/2000, strain Miyagi/9/96, strain Nara/2/85, strain NewJersey/76, strain pig/Beijing/115/81, strain Saitama/3/2000), strain Shizuoka/79, strain Yamagata/2/98, strain Yamagata/6/2000, strain Yamagata/9/96, strain BERLIN/1/85, strain ENGLAND/892/8, strain GREAT LAKES/1167/54, strain JJ/50, strain PIG/BEIJING/10/81, strain PIG/BEIJING/439/82), strain TAYLOR/1233/47, and strain C/YAMAGATA/10/81.

In some embodiments, the RNA virus is a hepatitis virus, including hepatitis A, C, D, and E virus. In some embodiments, the RNA virus is a hepatitis C virus. Non-limiting examples of HCV virus include genotype 1, genotype 2, genotype 3, genotype 4, genotype 5, and genotype 6. Each genotype has several subtypes, which are represented by lowercase letter (e.g., 1a, 1b, 2a, etc.), encompassing 18 subtypes (1a, 1b, 2a to 2c, 3a to 3c, 4a to 4 h, 5a, and 6a). The genotypes of HCV show a distinct geographical distribution. Genotypes 1, 2, and 3 are globally distributed. Genotype 4 is the predominant genotype of the Middle East and Africa. Types 5 and 6 are largely confined to South Africa and South East Asia, respectively. HCV genotype 3 is particularly prevalent in intravenous drug abusers in Europe and the United States. In Korea, genotypes 1 and 2 account for more than 99% of HCV species, genotype 3 seems to be found uncommonly (<1%).

### Additional Therapeutic Agents

The methods disclosed herein can comprise administering to the subject in need thereof one or more additional therapeutic agents (e.g., antiviral agents). The additional therapeutic agents (e.g., antiviral agents) can be co-administered to the subject with the composition. The additional therapeutic agents can be administered to the subject before the administration of the composition, after the administration of the composition, or both. The composition can comprise one or more additional therapeutic agents.

The antiviral agent can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

As disclosed herein, co-administration of particular ratios and/or amounts of a compound of the disclosure (e.g., a compound listed in Table 1, or a pharmaceutically acceptable salt, ester, solvate, stereoisomer, tautomer, or prodrug thereof) and one or more additional therapeutic agents (e.g., antiviral agents) can result in synergistic effects in preventing, delaying the onset of, or treating an infection, disease, or inflammatory effect caused by a RNA virus. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be, be about, be greater than, or be greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is, for example, a reduction in lung inflammation and that lung inflammation is reduced to a greater degree as compared to the sum of the effects of administering each component, determined as if each component exerted its effect independently, also referred to as the predicted additive effect herein. For example, if a composition comprising component (a) yields an effect of a 20% reduction in lung inflammation and a composition comprising component (b) yields an effect of 50% reduction in lung inflammation, then a composition comprising both component (a) and component (b) would have a synergistic effect if the combination composition's effect on lung inflammation was greater than 70%.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least, or at least about, 20, 50, 75, 90, 100, 150, 200 or 300% greater than the predicted additive effect. In some embodiments, the synergistic effect can be at least, or at least about, 0.2, 0.5, 0.9, 1.1, 1.5, 1.7, 2, or 3 fold greater than the predicted additive effect.

In some embodiments, the synergistic effect of the combination compositions can also allow for reduced dosing amounts, leading to reduced side effects to the subject and reduced cost of treatment. Furthermore, the synergistic effect can allow for results that are not achievable through any other treatments. Therefore, proper identification, specification, and use of combination compositions can allow for significant improvements in the reduction and prevention of lung inflammation.

The additional therapeutic agents can include antagonists of transient receptor potential cation channels, including but not limited to transient receptor potential ankyrin A1 (TRPA1) antagonists. The additional therapeutic agents provided herein can include TRPV1 agonists that cause decrease in TRPV1 activity (desensitization) upon prolonged exposure of TRPV1 to the stimuli, including but not limited to capsaicin, camphor, carvacrol, menthol, methyl salicylate, resiniferatoxin, tinyatoxin, and analogs, derivatives and salts thereof.

The additional therapeutic agents can include antagonists of protease-activated receptors (PARs) and inhibitors of activating proteases. The additional therapeutic agents can include antagonists of endothelin receptors, including but not limited to selective endothelin A receptor (ETAR) antagonists. The additional therapeutic agents provided herein can include inhibitors of Toll-like receptors (TLRs). The additional therapeutic agents can include inhibitors of mitogen-activated protein (MAP) kinases. The additional therapeutic agents can include inhibitors of mitogen-activated protein kinase kinases (MEKs).

The additional therapeutic agents can include inhibitors of calcitonin gene-related peptide (CGRP) or receptor therefor or the production thereof. The additional therapeutic agents can include inhibitors of gastrin-releasing peptide (GRP) or the receptor therefor (GRPR, aka bombesin receptor 2 [BBR2]) or the production thereof, including but not limited to CRPR antagonists (e.g.; RC-3095), and analogs, derivatives and salts thereof.

The additional therapeutic agents provided herein can include inhibitors of nerve growth factor (NGF) or receptors therefor tropomyosin kinase receptor A [TrkA]) or the production thereof, including but not limited to NGF inhibitors (e.g., fulranumab and tanezumab), NGF receptor inhibitors (e.g., TrkA inhibitors such as A0879, CT327 and K252a), and analogs, derivatives, fragments and salts thereof. The additional therapeutic agents provided herein can include inhibitors of neurotensin or receptors therefor (e.g., neurotensin receptor 1 [NTSR1], NTSR2 and so 1) or the production thereof, including but not limited to selective NTSR1 antagonists (e.g., SR-48,692), selective NTSR2 antagonists (e.g., levocabastine), unselective receptor antagonists (e.g., SR-142,948), and analogs, derivatives and salts thereof.

The additional therapeutic agents provided herein can include inhibitors of somatostatin or receptors therefor (e.g., somatostatin receptors [SSTRs]1 to 5) or the production thereof, including but not limited to selective SSTR2 antagonists (e.g., CYN 154806), selective SSTRS antagonists (e.g., BIM 23056), unselective SSTR antagonists (e.g., cyclosomatostatin), and analogs, derivatives, fragments and salts thereof. The additional therapeutic agents provided herein can include inhibitors of vasoactive intestinal peptide (VIP) or receptors therefor (e.g., VIPR1 and VIPR2) or the production thereof, including but not limited to VIP receptor antagonists {e.g., PG 97-269, ViPhyb, VIP(6-28)-NH₂, [p-Cl-D-Phe⁶, Leu¹⁷]VIP—NH₂, [Ac-His¹, D-Phe², Lys¹⁵, Arg¹⁶]VIP(3-7)GRF(8-27)-NH₂, and [Ac-Tyr¹, D-Phe²]GRF(1-29)-NH₂}, and analogs, derivatives, fragments and salts thereof. The additional therapeutic agents provided herein can include inhibitors of bradykinin or receptors therefor (e.g., B1 and B2) or the production thereof, including but not limited to bradykinin inhibitors (e.g., aloe, bromelain and polyphenols), bradykinin receptor B2 antagonists (e.g., icatibant and FR-173657), inhibitors of kallikreins (e.g., ecallantide, camostat, nafamostat, gabexate and C1-inhibitor), and analogs, derivatives and salts thereof. The additional therapeutic agents provided herein can include inhibitors of corticotropin-releasing hormone (CRH, aka corticoliberin) or receptors therefor (e.g., CRHR1 and CRHR2) or the production thereof, including but not limited to CRHR1 antagonists (e.g., antalarmin, pexacerfont, CP-154,526 LWH-234, NBI-27914 and R-121,919), CRHR2 antagonists (e.g., astressin-B), and analogs, derivatives and salts thereof.

The additional therapeutic agents provided herein can include antihistamines, including but not limited to antihistamines that inhibit action at the histamine H₁ receptor (e.g., acrivastine, antazoline, astemizole, azatadine, azelastine, bepotasiine, bilastine, bromodiphenhydramine, brompheniramine, buclizine, carbinoxamine, cetirizine, chlorcyclizine, chlorodiphenhydramine, chlorpheniramine, chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-enantiomer of mirtazapine], fexofenadine, hydroxyzine, ketotifen, levocabastine, levocetirizine, loratadine, meclozine mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine tripelennamine and triprolidine), antihistamines that inhibit action at the histamine H₃ receptor (e.g., betahistine, burimamide, ciproxifan, clobenpropit, conessine, failproxifan, impentamine, iodophenpropit, irdabisant, pitolisant, thioperamide, A-349,821, ABT-239 and VUF-568), antihistamines that inhibit action at the histamine H₄ receptor (e.g., clobenpropit, thioperamide, A943931, A987306, JNJ-7777120, VUF-6002 and ZPL-389), and analogs, derivatives and salts thereof.

The additional therapeutic agents provided herein can include inhibitors of phospholipase A2 (e.g., secreted and cytosolic PLA2), including but not limited to arachidonyl trifluoromethyl ketone, bromoenol lactone, chloroquine, cytidine 5-diphosphoamines, darapladib, quinacrine, vitamin E, RO-061606, ZPL-521, lipocortins (annexins), and analogs, derivatives, fragments and salts thereof.

The additional therapeutic agents provided herein can include inhibitors of pro-inflammatory prostaglandins (e.g., prostaglandin E2) or receptors therefor or the production thereof, including but not limited to non-steroidal anti-inflammatory drugs (NSAIDs) (e.g., non-selective COX-1/COX-2 inhibitors such as aspirin and selective COX-2 inhibitors such as coxibs), glucocorticoids, cyclopentenone prostaglandins (e.g., prostaglandin J2 [PGJ2], Δ12-PGJ2 and 15-deoxy-Δ12,14-PGJ2), and analogs, derivatives and salts thereof, inhibitors of leukotrienes or receptors therefor or the production thereof, including but not limited to leukotriene receptor antagonists (e.g., cinalukast, gemilukast, iralukast, montelukast, pranlukast, tomelukast, verlukast, zafirlukast, CP-199330, HAMI-3379, ICI-198615 and MK-571), 5-lipoxygenase inhibitors (e.g., baicalein, caffeic acid, curcumin, hyperforin, meclofenamic acid, meclofenamate sodium, zileuton and MK-886), and analogs, derivatives and salts thereof.

The additional therapeutic agents provided herein can include mast cell stabilizers, including but not limited to cromoglicic acid (cromolyn), ketotifen, methylxanthines, nedocromil, olopatadine, omalizumab, pemirolast, quercetin. 02-adrenoreceptor agonists {including short-acting 02-adrenergic agonists (e.g., bitolterol, fenoterol, isoprenaline [isoproterenol], levosalbutamol [levalbuterol], orciprenaline [metaproterenol], pirbuterol, procaterol, ritodrine, salbutamol [albuterol] and terbutaline), long-acting β₂-adrenergic agonists arformoterol, bambuterol, clenbuterol, formoterol and salmeterol), and ultralong-acting β₂-adrenergic agonists (e.g., carmoterol, indacaterol, milveterol, olodaterol and vilanterol)}, and analogs, derivatives and salts thereof.

The additional therapeutic agents can include Janus kinase (JAX) inhibitors, including, but not limited to JAK1 inhibitors (e.g., GLPG0634 and GSK2586184). JAK2 inhibitors, dual JAK1/JAK2 inhibitors (e.g., baricitinib and ruxolitinib), dual JAK1/JAK3 inhibitors (e.g., tofacitinib), and analogs, derivatives and salts thereof.

The additional therapeutic agents can include immunomodulators, including but not limited to imides (e.g., thalidomide, lenalidomide, pomalidomide and apremilast), xanthine derivatives (e.g., lisofylline, pentoxifylline and propentofylline), and analogs, derivatives and salts thereof. The additional therapeutic agents can include immunosuppressants, including but not limited to glucocorticoids, antimetabolites (e.g., hydroxyurea [hydroxycarbamide], antifolates [e.g., methotrexate], and purine analogs [e.g., azathioprine, mercaptopurine and thioguanine]), calcineurin inhibitors (e.g, ciclosporin [cyclosporine A], pimecrolimus and tacrolimus), inosine-5′-monophosphate dehydrogenase (IMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9], zotarolimus and RTP-801), modulators of sphingosine-1-phosphate receptors (e.g., SIPR1) (e.g., fingolimod), serine C-palmitoyltransferase inhibitors (e.g., myriocin), and analogs, derivatives and salts thereof. The additional therapeutic agents can include corticosteroids/glucocorticoids. The additional therapeutic agents can include inhibitors of pro-inflammatory cytokines or receptors therefor, including but not limited to inhibitors of (e.g., antibodies to) tumor necrosis factor-alpha (TNF-α) (e.g, adalimumab, certolizumab pegol, golimumab, infliximab, etanercept, bupropion and ART-621), inhibitors of (e.g., antibodies to) pro-inflammatory interferons (e.g., interferon-alpha [IFN-α]) or receptors therefor, inhibitors of (e.g., antibodies to) pro-inflammatory interleukins or receptors therefor (e.g., IL-1 [e.g., IL-1α and IL-1β] or IL-1R [e.g., EBI-005 {isunakinra}], IL-2 or IL-2R [e.g., basiliximab and daclizumab], IL-4 or IL-4R [e.g., dupilumab], IL-5 [e.g., mepolizumab] or IL-5R, IL-6 [e.g., clazakizumab, elsilimomab, olokizumab, siltuximab and sirukumab] or IL-6R [e.g., sarilumab and tocilizumab], IL-8 or IL-8R, IL-12 [e.g., briakinumab and ustekinumab] or IL-12R, IL-13 or IL-13R, IL-15 or IL-15R, IL-17 [e.g., ixekizumab and secukinumab] or IL-17R [e.g., brodalumab], IL-18 or IL-18R, IL-20 [e.g., the antibody 7E] or IL-20R, IL-22 [e.g., fezakinumab] or IL-22R, IL-23 [e.g., briakinumab, guselkumab, risankizumab, tildrakizumab SCH-9002221, ustekinumab and BI-655066] or IL-23R, IL-31 or IL-31R [e.g., anti-IL-31 receptor A antibodies such as nemolizumab], IL-33 or IL-33R, and IL-36 or IL-36R), and analogs, derivatives, fragments and salts thereof.

The additional therapeutic agents can include inhibitors of the production of pro-inflammatory cytokines or receptors therefor, including but not limited to inhibitors of the production of TNF-α (e.g., myxoma virus M013 protein, Yersinia YopM, protein, glucocorticoids, immunomodulatory imides, PDE4 inhibitors, p38 MAP kinase inhibitors, inhibitors of TLRs such as TLR7 and TLR9, scrim protease inhibitors [e.g., gabexate and nafamostat], and prostacyclin, carbacyclin and analogs and derivatives thereof [e.g., beraprost, cicaprost, ciprosten, eptaloprost, iloprost and treprostinil]), IFN-α (e.g., alefacept and inhibitors of TLRs such as TLR7 and TLR9), IL-1 (e.g., IL-1α, and IL-10) (e.g., M013 protein, YopM protein, nafamostat, prostacyclin, glucocorticoids, TNF-α inhibitors, inhibitors of TLRs such as TLR7 and TLR9, and PAR1 antagonists), IL-2 (e.g., glucocorticoids, calcineurin inhibitors and PDE4 inhibitors), IL-4 (e.g., glucocorticoids and serine protease inhibitors [e.g., gabexate and nafamostat]), IL-5 (e.g., glucocorticoids), IL-6 M013 protein, nafamostat, prostacyclin, tranilast, glucocorticoids, immunomodulatory imides, TNF-α inhibitors, and inhibitors of TLRs such as TLR7 and TLR9), IL-8 alefacept, glucocorticoids and PAR2 antagonists [e.g., tetracyclines]), IL-12 (e.g., apilimod, YopM protein, PDE4 inhibitors, and inhibitors of TLRs such as TLR7 and TLR9), IL-15 (e.g., YopM protein), IL-17 (e.g., protein kinase C [PKC] inhibitors such as sotrastaurin), IL-18 (e.g., MOD protein and YopM protein), and IL-23 (e.g., apilimod, alefacept and PDE4 inhibitors), and analogs, derivatives, fragments and salts thereof.

The additional therapeutic agents can include other kinds of anti-inflammatory agents, including but not limited to inhibitors of pro-inflammatory transcription factors e.g., inhibitors of NE-κB [e.g., nafamostat, M013 protein, penetranin, (−)-DHMEQ, IT-603, IT-901 and PBS-1086] and inhibitors of STAT [signal transducer and activator of transcription] proteins [e.g., JAK1, JAK2 and JAK3 inhibitors]), antagonists of the prostaglandin D2 receptor (DP₁) or/and the chemoattractant receptor homologous molecule expressed on TH₂ cells (CRTH2) (e.g., TS-022), phosphodiesterase (PDE) inhibitors (e.g., PDE4 inhibitors such as apremilast, cilomilast, ibudilast, piclamilast, roflumilast, crisaborole, diazepam, luteolin, mesembrenone, rolipram, AN2728 and E6005), IgE inhibitors (e.g., anti-IgE antibodies such as omalizumab), myeloperoxidase inhibitors (e.g., dapsone), specialized pro-resolving mediators (SPMs) (e.g., metabolites of polyunsaturated fatty acids such as lipoxins, resolvins [including resolvins derived from 5Z,8Z,11Z,14Z,17Z-eicosapentaenoic acid {EPA}, resolvins derived from 4Z,7Z,10Z,13Z,16Z,19Z-docosahexaenoic acid {DHA}, and resolvins derived from 7Z,10Z,13Z,16Z,19Z-docosahexaenoic acid {n-3 DPA}], protectins/neuroprotectins [including DHA-derived protectins/neuroprotectins and n-3 DPA-derived protectins/neuroprotectins], maresins [including DHA-derived maresins and n-3 DPA-derived maresins], n-3 DPA metabolites, n-6 DPA {4Z,7Z,10Z,13Z,16Z-docosapentaenoic acid}metabolites, oxo-DHA metabolites, oxo-DPA metabolites, docosahexaenoyl ethanolamide metabolites, cyclopentenone prostaglandins [e.g., Δ12-PGJ2 and 15-deoxy-Δ12,14-PGJ2], and cyclopentenone isoprostanes [e.g., 5,6-epoxyisoprostane A2 and 5,6-epoxyisoprostane E2]), disease-modifying antirheumatic drugs (DMARDs, e.g., sulfasalazine and mesalazine [5-aminosalicylic acid]), anti-allergic agents (e.g., antihistamines, inhibitors of leukotrienes or receptors therefor or the production thereof, mast cell stabilizers, glucocorticoids, epinephrine [adrenaline] and tranilast), ultraviolet radiation (e.g., ultraviolet A and B), and analogs, derivatives, fragments and salts thereof. The additional therapeutic agents can include antagonists of serotonin receptors, antagonists of muscarinic acetylcholine receptors (e.g., M1 to M5).

The one or more antiviral agents and/or the one or more additional therapeutic agents can one or more of the following: Gimsilumab, an anti-granulocyte-macrophage colony stimulating factor monoclonal antibody, a non-viral gene therapy producing monoclonal antibodies, EB05, a non-steroidal anti-inflammatory molecule (sPLA2 inhibitor), Opdivo (nivolumab), a PD-1 blocking antibody, IC14, a recombinant chimeric anti-CD14 monoclonal antibody, avastin (bevacizumab), a vascular endothelial growth factor inhibitor, a PD-1 blocking antibody, Thymosin, meplazumab, an anti-CD147 antibody, an antibody combination REGN-COV2 (REGN10933+REGN10987) against the spike protein MEDI3506, a monoclonal antibody targeting interleukin 33, OmniChicken platform antibodies, antibodies from recovered COVID-19 patients, Antibody 47D11, Polyclonal hyperimmune globulin (H-IG), LY-CoV555 antibody, otilimab, an anti-granulocyte macrophase colony-stimulating factor (GM-CSF) antibody, LY3127804, an anti-Angiopoietin 2 (Ang2) antibody, a CXC10 antagonist, polyclonal hyperimmune globulin (H-IG), Octagam, intravenous Immunoglobulin (IVIG), single domain antibodies (sdAbs), an engineered monoclonal antibody derived from camelids, a super-antibody or antibody cocktail to target potential mutations of SARS-CoV-2, AiRuiKa (camrelizumab), an anti-programmed cell death protein (PD-1) antibody, Linked nanobody antibody, antibodies from recovered COVID-19 patients, OmniRat platform antibodies, Soliris (eculizumab), a complement inhibitor, CT-P59, Ultomiris (ravulizumab-cwvz), rCIG (recombinant anti-coronavirus 19 hyperimmune gammaglobulin), VIR-7831, VIR-7832, Gamifant (emapalumab), an anti-interferon gamma antibody, leronlimab (PRO 140), an CCR5 antagonist, polyclonal hyperimmune globulin (H-IG), Sylvant (siltuximab), an interleukin-6 targeted monoclonal antibody, Actemra (tocilizumab), an interleukin-6 receptor antagonist, Kevzara (sarilumab), an interleukin-6 receptor antagonist, purified ovine immunoglobulin from immunized sheep, lenzilumab, an anti-granulocyte-macrophage colony stimulating factor antibody, Ilaris (canakinumab), an interleukin-1beta blocker, JS016 antibody, TJM2 (TJ003234), an anti-granulocyte-macrophage colony stimulating factor antibody, COVI-SHIELD antibody cocktail, an antibody targeting the S protein, COVID-EIG plasma, SAB-185, polyclonal hyperimmune globulin (H-IG), IFX-1, an anti-C5a antibody, CERC-002, an anti-LIGHT monoclonal antibody, Remsima (infliximab), an anti-TNF antibody, TY027, a monoclonal antibody targeting SARS-CoV-2, IgY-110, an anti-CoV-2 antibody (nasal spray application), mavrilimumab, an anti-granulocyte-macrophase colony-stimulating factor receptor-alpha monoclonal antibody, BDB-100, monocloncal anti-C5a antibody, TZLS-501, an anti-interleukin-6 receptor monoclonal antibody, itolizumab, anti-CD6 IgG1 monoclonal antibody, GC5131A, BTL-tml, galidesivir, emetine hydrochloride, DAS181, recombinant sialidase (nebulized), Favilavir/Favipiravir/T-705/Avigan, Vicromax, ISR-50, Levovir (clevudine), AB001, EIDD-2801, an oral ribonucleoside analog, ASC09, an HIV protease inhibitor, Tamiflu (oseltamivir), a neuraminidase inhibitor, Truvada, emtricitabine, tenofovir, a HIV-1 nucleoside analog reverse transcriptase inhibitor, Virazole, ribavirin for inhalation solution, AT-527, an oral purine nucleotide prodrug, Ganovo (danoprevir), a hepatitis C virus NS3 protease inhibitor, ritonavir, remdesivir, a nucleotide analog, Arbidol (umifenovir), Prezcobix (darunavir, HIV-1 protease inhibitor/cobicistat, CYP3A inhibitor), Kaletra/Aluvia (lopinavir/ritonavir), an HIV-1 protease inhibitor, prophylactic antiviral CRISPR in human cells (PAC-MAN), GC376, AmnioBoost, concentrated allogeneic MSCs and cytokines derived from amniotic fluid, Astrostem-V, allogenic adipose-derived mesenchymal stem cells (HB-adMSCs), bone marrow-derived allogenic mesenchymal stem cells (BM-Allo-MSC), mesenchymal stem cells, allogenic adipose-derived mesenchymal stem cells (HB-adMSCs) haNK, natural killer cells, Ryoncil (remestemcel-L), allogenic mesenchymal stem cells, MultiStem, bone marrow stem cells, allogeneic T-cell therapies, Autologous Adipose-Tissue Derived Mesenchymal Stem Cells (ADMSCs) and allogeneic MSCs, CYNK-001, CAP-1002, allogenic cardiosphere-derived cells, PLX cell product, placenta-based cell therapy, Chimeric antigen receptors (CAR)/T cell receptors (TCR)-T cell therapy, natural killer cell-based therapy, small mobile stem (SMS) cells, IMS001, human embryonic stem cell-derived mesenchymal stem cells (hES-MSC), VIR-2703 (ALN-COV) siRNA, OT-101, a TGF-Beta antisense drug, inhaled mRNA, peptide conjugated antisense oligonucleotides, Ampligen, rintatolimod, BXT-25, glycoprotein, EDP1815, Ivermectin, tradipitant, a neurokinin-1 receptor antagonist, piclidenoson, A3 adenosine receptor agonist, Ryanodex (dantrolene sodium), a skeletal muscle relaxant, Jakafi/jakavi (ruxolitinib), nitazoxanide, antiprotozoal, peptides targeting the NP protein, interferon/peginterferon alpha-2b, PegIntron, Sylatron, IntronA, PegiHep, roscovitine seliciclib, cyclin-dependent kinase (CDK)2/9 inhibitor, ATYR1923, a fusion protein comprising immuno-modulatory domain of histidyl tRNA synthetase fused to the Fc region of a human antibody, a modulator of neuropilin-2, Leukine (sargramostim, rhu-Granulocyte macrophage colony stimulating factor), ADX-1612, HSP 90 inhibitor, DSTAT (dociparstat sodium), glycosaminoglycan derivative of heparin, BIO-11006, Recombinant human interferon alpha-1b, ST-001 nanoFenretinide (fenretinide), Activase (alteplase), tissue plasminogen activator (tPA), camostat mesylate, a transmembrane protease serine 2 (TMPRSS2) inhibitor, nitric oxide, Cozaar (losartan), an angiotensin II receptor blocker (ARB), Otezla (apremilast), an inhibitor of phosphodiesterase 4 (PDE4), IMU-838, a selective oral dihydroorotate dehydrogenase (DHODH) inhibitor, Colchicine, Brilacidin, a defensin mimetic, Metablok (LSALT peptide), a selective dipeptidase-1 antagonist, nafamostat, CD24Fc, an agent comprising nonpolymorphic regions of CD24 attached to the Fc region of human IgG1, Aplidin (plitidepsin), fadraciclib (CYC065), a cyclin-dependent kinase (CDK)2/9 inhibitor, Aviptadil, a synthetic form of Vasoactive Intestinal Polypeptide (RLF-100), solnatide, a synthetic molecule with a structure based on the lectin-like domain of human Tumour Necrosis Factor alpha, PP-001, MRX-4DP0004, a strain of Bifidobacterium breve isolated from the gut microbiome of a healthy human, ARMS-1, BLD-2660, a small molecule inhibitor of calpain (CAPN) 1, a small molecule inhibitor of CAPN2, a small molecule inhibitor of CAPN9, LAU-7b (fenretinide), N-803, an IL-15 “superagonist” (Nogapendekin alfa inbakicept), Rebif, interferon beta-1a, DIBI, an iron-binding polymer, EPAspire, an oral formulation of highly purified eicosapentaenoic acid free fatty acid (EPA-FFA) in gastro-resistant capsules, MN-166 (ibudilast), a small molecule macrophase migration inhibitory factor (MIF) inhibitor, a phosphodiesterase (PDE) 4 inhibitor, a PDE10 inhibitor, ADX-629, an orally available reactive aldehyde species (RASP) inhibitor, Calquence (acalabrutinib), a Bruton's tyrosine kinase (BTK) inhibitor, Auxora (CM4620-IE), a calcium release-activated calcium (CRAC) channel inhibitor Neumifil, a multivalent carbohydrate binding molecule, Diovan (valsartan), an angiotensin II receptor blocker (ARB), Yeliva (opaganib, ABC294640), an oral sphingosine kinase-2 (SK2) selective inhibitor, WP1122, a glucose decoy prodrug, Kineret (anakinra), an interleukin-1 receptor antagonist, a microbiome therapeutic, Coronzot, bemcentinib, a selective AXL kinase inhibitor, a synthesized nanoviricide drug, Chloroquine/Hydroxychloroquine, an antimalarial drug Senicapoc, vazegepant, a CGRP receptor antagonist, APN01, a recombinant soluble human Angiotensin Converting Enzyme 2, GP1681, a small molecule inhibitor of cytokine release, ST266, a cell-free biologic made from anti-inflammatory proteins secreted by placental cells, recombinant human plasma gelsolin (rhu-pGSN), pacritinib, an oral kinase inhibitor with specificity for JAK2, IRAK1 and CSFIR, Ruconest (recombinant human C1 esterase inhibitor), Cerocal (ifenprodil), NP-120, an NDMA receptor glutamate receptor antagonist targeting Glu2NB, Peginterferon lambda, Pepcid (famotidine), a histamine-2 (H2) receptor antagonist, heparin, a low molecular weight heparin (enoxaparin), an anticoagulant, Xeljanz (tofacitinib), a Janus kinase (JAK) inhibitor, Xpovio (selinexor), a selective inhibitor of nuclear export (SINE) compound, a pH barrier, transepithelial nebulized alkaline treatment, Luvox (fluvoxamine), a selective serotonin reuptake inhibitor, Micardis (telmisartan), brensocatib, a reversible inhibitor of dipeptidyl peptidase 1 (DPP1) Novaferon, RHB-107 (upamostat, WX-671), a serine protease inhibitor, UNI9011, FW-1022, DWRX2003, niclosamide, Lysteda/Cyklokapron/LB1148 (tranexamic acid), an antifibrinolytic PUL-042 inhalation solution, ABX464, Gleevac (imatinib), Traumakine (interferon beta 1-a), Veyonda (idronoxil), Farxiga (dapagliflozin), a sodium-glucose cotransporter 2 (SGLTs) inhibitor, Gilenya (fingolimod), a sphingosine 1-phosphate receptor modulator, sPIF, a synthetic pre implantation factor, SNG001, an inhaled formulation of interferon beta-1a, Methylprednisolone, ciclesonide (Alvesco), hydrocortisone, corticosteroids Olumiant (baricitinib), a Janus kinase (JAK) inhibitor, dipyridamole (Persantine), an anticoagulant, AT-001, an aldose reductase inhibitor, Vascepa (icosapent ethyl), a form of eicosapentaenoic acid, OP-101, a dendrimer-based therapy, apabetalone (RVX-208), a selective BET (bromodomain and extra-terminal) inhibitor, Flarin (lipid ibuprofen), Almitrine, VPO1, an Angiotensin II Type 2 receptor activator, leflunomide, a pyrimidine synthesis inhibitor, Pulmozyme (nebulised dornase alfa), a recombinant DNase enzyme, AQCH, MSTT1041A (anti-ST2, the receptor for IL-33), UTTR1147A (IL-22-Fc), CIGB-258, FSD-201, ultramicronized palmitoylethanolamide, PB1046, a long-acting sustained release human vasoactive intestinal peptide (VIP) analogue, PTC299, an oral small molecule inhibitor of dihydroorotate dehydrogenase (DHODH), raloxifene (Evista), an estrogen agonist/antagonist, losmapimod, an oral selective p38 mitogen activated protein kinase inhibitor, dutasteride, an anti-androgen, M5049, small molecule capable of blocking the activation of Toll-like receptor (TLR)7 and TLR8, Eritoran, a TLR-4 antagonist, desidustat, a hypoxia inducible factor prolyl hydroxylase inhibitor, merimepodib, an IMPDH inhibitor, azithromycin, Cenicriviroc, a chemokine receptor 2 and 5 dual antagonist, Firazyr (icatibant), a bradykinin B2 antagonist, Razoprotafib, Tie 2 activating compound (AKB-9778), or any combination thereof.

Antiviral agents provided include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; palivizumab; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin. In certain aspects, other antibodies against viral proteins or cellular factors may be used in combination with a therapeutic composition described herein.

Antibacterial agents provided herein include, but are not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines. Anti-bacterials also include, but are not limited to: Acedapsone, Acetosulfone Sodium, Alamecin, Alexidine, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefepime, Cefepime Hydrochloride, Cefetecol, Cefixime, Cefinenoxime Hydrochloride, Cefinetazole, Cefinetazole Sodium, Cefonicid Monosodium, Cefonicid Sodium, Cefoperazone Sodium, Ceforanide, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexii Hydrochloride, Cephaloglycini, Cephaloridine, Cephalothin Sodium, Cephapirin Sodium, Cephradine, Cetocycline Hydrochloride, Cetophenicol, Chloramphenicol, Cliloramphenicol Palmitate, Chloramphenicol Pantotheniate Complex, Chloramphenicol Sodium Succinate, Chlorhexidine Phosphanilate, Chloroxylenol, Chlortetracycline Bisulfate, Chlortetracycline Hydrochloride, Cinoxacin, Ciprofloxacin, Ciprofloxacin Hydrochloride, Cirolemycin, Clarithromycin, Clinafloxacin Hydrochloride, Clildamycin, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate Sodium, Colistin Sulfate, Coumermycin, Coumermycin Sodium, Cyclacillin, Cycloserine, Dalfopristin, Dapsone, Daptomycin, Demeclocycine, Demeclocycine Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Droxacin Sodium, Enoxacin, Epicillin, Epitetracycline Hydrochloride, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofuraltadone, Levopropylcillin Potassium, Lexithromycin, Lincomycin, Lincomycin Hydrochloride, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifuirpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Streptomycin Sulfate, Streptonicozid, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfas alazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Tri sulfapyrimidines, Troleandomycin, Trospectomycin Sulfate, Tyrothricin, Vancomycin, Vancomycin Hydrochloride, Virginiamycin, and/or Zorbamycin.

Anti-fungal agents provided herein include, but are not limited to, azoles, imidazoles, polyenes, posaconazole, fluconazole, itraconazole, amphotericin B, 5-fluorocytosine, miconazole, ketoconazole, Myambutol (Ethambutol Hydrochloride), Dapsone (4,4′-diaminodiphenylsulfone), Paser Granules (aminosalicylic acid granules), rifapentine, Pyrazinamide, Isoniazid, Rifadin IV, Rifampin, Pyrazinamide, Streptomycin Sulfate and Trecator-SC (Ethionamide) and/or voriconazole (Vfend™).

### EXAMPLES

Some aspects of the embodiments discussed above are disclosed in further detail in the following examples, which are not in any way intended to limit the scope of the present disclosure.

### Example 1

### Broad-Spectrum Antiviral Activity of Pipendoxifene

This example shows that pipendoxifene is a broad-spectrum antiviral small molecule inhibitor because the Thumb pocket 1 region in viral RNA-dependent RNA polymerase (RdRp) is well-conserved across many pathogenic viruses.

### FBS Studies on Pipendoxifene

Protein binding of pipendoxifene was studied using rapid equilibrium dialysis (RED) method as described by van Liempd et al, Development and validation of Higher-Throughput Equilibrium Dialysis Assay for Protein Binding. J. Lab Autom. 16(1): 56-67 (2011). The protein bound, and unbound fractions observed for pipendoxifene are shown in Table 2.

The protein binding study results showed that pipendoxifene's free concentration in in vitro studies is much lower than its total concentration. Without being bound by any particular theory, it is believed that FBS, which is a significant component of cell assays, lowers the free concentration of pipendoxifene's by 75% at 2% FBS and 95% at 10% FBS. One of the resulting effects is that the observed potency/EC50 numbers can be “normalized” by dividing the EC50 concentration by 20 for the HCV assays (10% FBS) and by 4 for most other assays (2% FBS). Therefore, the results shown below in Table 2 are the “normalized EC50” numbers that are much lower than “observed EC50” numbers. For 2% FBS, an unbound fraction of 25% was observed (and thus a 4× reduction from the apparent EC50), and for 10% FBS, an unbound fraction of 5% was observed (and thus a 20× reduction from apparent EC50).

TABLE 2
Results of protein binding experiments for pipendoxifene,
summary of the mean (n = 3) unbound and bound
percentages, and the compound recovery.

| | | | Mean | Mean | | Mean |
|---|---|---|---|---|---|---|
| | | Conc. | unbound | bound | Std. | recov- |
| Compound | FBS | (μM) | (F_{ub}, %) | (F_{b}, %) | dev. | ery (%) |
| Pipendoxifene | 2% heat | 1 | 25.0 | 75.0 | 3.5 | 106.6 |
| | inactivated | | | | | |
| | 10% heat | 1 | 5.0 | 95.0 | 0.5 | 102.7 |
| | inactivated | | | | | |
| | 2% not | 1 | 24.4 | 75.6 | 4.2 | 91.2 |
| | inactivated | | | | | |
| | 10% not | 1 | 4.7 | 95.3 | 1.1 | 112.4 |
| | inactivated | | | | | |

### Antiviral Activity of Pipendoxifene

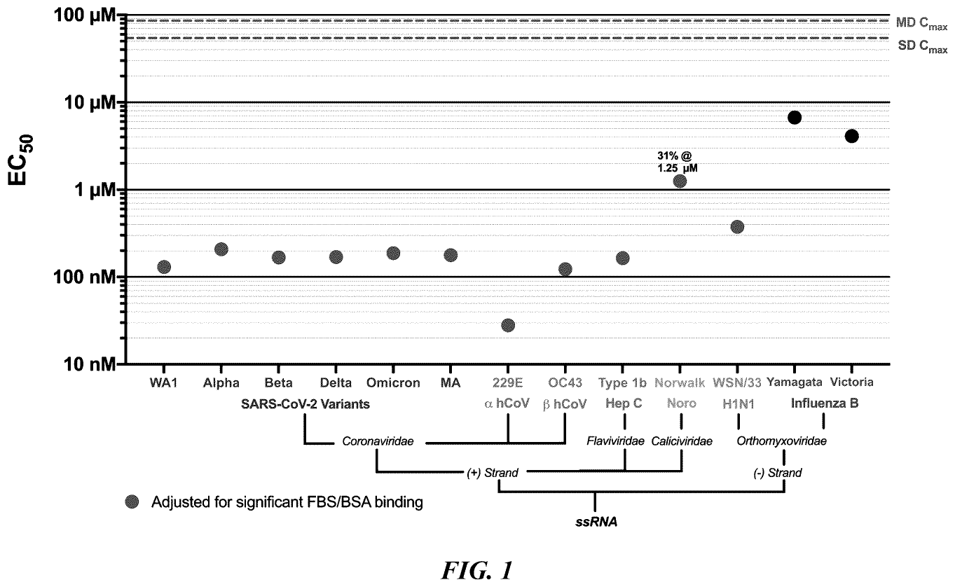

In vitro potency of pipendoxifene was tested in a panel of 13 cell line-based virus expression assays. These included 10+ssRNA viruses (six SARS-CoV-2 variants, two coronavirus types, HCV and NoV), and 2-ssRNA viruses (IAV-H1N1 and IAB). Pipendoxifene had submicromolar potency against all six SARS-CoV-2 variants (Wuhan isolate WA1, Alpha, Beta, Delta, and Omicron variants, as well as a mouse-adapted variant SARS-CoV-2 MA), and the two coronaviruses tested (Alphacoronavirus and Betacoronavirus). The results are shown in FIG. 1, demonstrating that pipendoxifene had significant potency (EC50=3-8 μM) against the remaining+ssRNA viruses (HCV, and NoV), and the -ssRNA viruses (IAV and JAB).

In vitro antiviral potency of pipendoxifene was tested with a panel of 14 viral variants from eight pathogenic viruses across four ssRNA viral families. Coronaviridae (+ssRNA): SARS-CoV-2 variants, including Alpha, Beta, Delta, Omicron, the initial Wuhan isolate (WA1), a mouse-adapted (MA) variant, HCV and NoV. Flaviviridae. Orthomyxoviridae (-ssRNA): Influenza A H1N1/WSN/33 and Influenza B Yamagata/Brisbane and Victoria/Florida variants.

Antiviral potency was determined based on half maximal effective concentration (EC50) and 5000 cytotoxicity concentration (CC50), which in turn allowed us to determine the half maximal selective index (SI50), after dividing CC50 by EC50. The antiviral assays were cell line based and consisted in transfecting each of the viruses tested in a compatible cell line. Upon infection and after drug treatment, viral survival and cell toxicity were reported from the readout of three different assays: cytopathic effect (CPE), viral yield reduction (VYR), or immunofluorescence (IF). A breakdown of the results obtained can be found in Table 3 in which EC50 values have been converted to account for variations in FBS concentrations across assays.

TABLE 3
Breakdown of EC₅₀, CC₅₀, and SI₅₀ values across various viruses, assay types, and laboratories,
providing a holistic view of pipendoxifene's broad-spectrum antiviral results.

| | | | EC50 | Albumin | Corrected | CC50 | | Assay |
|---|---|---|---|---|---|---|---|---|
| | Strain | Cell Line | (μM) | content | EC50 (μM) | (μM) | SI50 | Type |
| Coronaviridae | | | | | | | | |
| SARS-CoV-2 | USA- | HeLa-ACE2 | 0.75 | 2% FBS¹ | 0.188 | >50 | >66.7 | IF |
| | WA1/2020 | | | | | | | |
| | USA- | HeLa-ACE2 | 0.675 | 2% FBS¹ | 0.169 | 7.652 | 11.3 | IF |
| | WA1/2020 | | | | | | | |
| | USA- | Vero E6 | 0.52 | 2% FBS² | 0.130 | 3.2 | 6.2 | CPE |
| | WA1/2020 | | | | | | | |
| | USA- | A549-ACE2 | 1.8 | 2% FBS² | 0.450 | >10 | >5.6 | CPE |
| | WA1/2020 | | | | | | | |
| | USA- | A549-ACE2 | 1.9 | 2% FBS² | 0.475 | >10 | >5.3 | CPE |
| | WA1/2020 | | | | | | | |
| | USA- | Caco-2 | 0.54 | 2% FBS² | 0.135 | 1 | 1.9 | VYR |
| | WA1/2020 | | | | | | | |
| | USA- | Vero 76 | 2.6 | 2% FBS² | 0.650 | 2.9 | 1.1 | CPE |
| | WA1/2020 | | | | | | | |
| | Alpha | HeLa-ACE2 | 0.83 | 2% FBS¹ | 0.208 | >50 | >60.2 | IF |
| | (B.1.1.7) | | | | | | | |
| | Alpha | HeLa-ACE2 | 1.197 | 2% FBS¹ | 0.299 | 7.652 | 6.4 | IF |
| | (B.1.1.7) | | | | | | | |
| | Beta | HeLa-ACE2 | 1.05 | 2% FBS¹ | 0.263 | >50 | >47.6 | IF |
| | (B.1.351) | | | | | | | |
| | Beta | HeLa-ACE2 | 0.668 | 2% FBS¹ | 0.167 | 7.652 | 11.5 | IF |
| | (B.1.351) | | | | | | | |
| | Delta | HeLa-ACE2 | 0.74 | 2% FBS¹ | 0.185 | >50 | >67.6 | IF |
| | (B.1.617.2) | | | | | | | |
| | Delta | HeLa-ACE2 | 0.677 | 2% FBS¹ | 0.169 | 7.652 | 11.3 | IF |
| | (B.1.617.2) | | | | | | | |
| | Delta | A549-ACE2 | 1.3 | 2% FBS² | 0.325 | >10 | >7.7 | CPE |
| | (B.1.617.2) | | | | | | | |
| | Delta | A549-ACE2 | 2.7 | 2% FBS² | 0.675 | >10 | >3.7 | CPE |
| | (B.1.617.2) | | | | | | | |
| | Delta | Vero 76 | 2.4 | 2% FBS² | 0.600 | 3.2 | 1.3 | CPE |
| | (B.1.617.2) | | | | | | | |
| | Omicron | HeLa-ACE2 | 0.75 | 2% FBS¹ | 0.188 | >50 | >66.7 | IF |
| | (B.1.1.529) | | | | | | | |
| | Omicron | HeLa-ACE2 | 1.037 | 2% FBS¹ | 0.259 | 7.652 | 7.4 | IF |
| | (B.1.1.529) | | | | | | | |
| | MA-WA1 | HeLa-ACE2 | 0.712 | 2% FBS¹ | 0.178 | 7.652 | 10.7 | IF |
| α-coronavirus | 229E | Huh7 | 0.42 | 2% FBS² | 0.105 | 8.4 | 20.0 | CPE |
| | 229E | MRC-5 | 0.11 | 2% FBS³ | 0.028 | 1.03 | 9.4 | CPE |
| | 229E | MRC-5 | 0.52 | 2% FBS³ | 0.13 | >1 | >1.9 | CPE |
| β-coronavirus | OC43 | A549-ACE2 | 0.49 | 2% FBS² | 0.123 | >15 | >30.6 | CPE |
| Flaviviridae | | | | | | | | |
| Hepatitis C Virus | Genotype | Huh-luc/neo- | 3.28 | 10% FCS⁴ | 0.164 | >10 | >3.0 | Replicon |
| | 1b | ET | | | | | | |
| Caliciviridae | | | | | | | | |
| Norovirus | Norwalk | HG23 | 31% @ | 2% FBS⁶ | 31% @ 1.25 | 18 | — | CPE |
| | Virus | | 5 μM | | μM | | | |
| Orthomyxoviridae | | | | | | | | |
| Influenza A | H1N1/WSN/ | A549 | 7.49 | 0.5% | 0.375 | 13.7 | 1.8 | IF |
| | 33 | | | BSA⁷ | | | | |
| Influenza B | Yamagata/ | MDCK | 6.7 | N/A | 6.7 | 9.8 | 1.5 | CPE |
| | Brisbane | | | | | | | |
| | Victoria/Florida | MDCK | 4.1 | N/A | 4.1 | 12 | 2.9 | CPE |

CPE: Cytopathic Effect,
VYR: Viral Yield Reduction,
IF: Immunofluorescence
* 2% FBS Converted
** 10% FBS Converted
*** 0.5% BSA~10% FBS equivalent (assuming standard FBS is 5 g/dL BSA = 5% BSA)
¹, ², ³, ⁴, ⁵, ⁶, and ⁷ - the assays were performed according to the corresponding methods 1-7 described below.
CC50 values not converted. SI values have not been modified from those obtained from raw results.
Because at 2% FBS, 25% of MDL-001 is unbound, EC50's in this condition are corrected by reducing it to 1/4 the apparent value.
Because at 10% FBS, 5% of MDL-001 is unbound, EC50's in this condition are corrected by reducing it to 1/20 the apparent value.
Because at 0.5% BSA, there is an equivalent amount of bovine albumin as a 10% FBS solution, the EC50 in this condition is corrected by reducing it to 1/20 the apparent value.

Descriptions for the methods (i.e., Methods 1-7) used for obtaining the assay results shown in Table 3 above are provided below:

### Method 1. SARS-CoV-2

Two thousand Vero-TMPRSS2 or HeLa-ACE2 cells were seeded into 96-well plates in DMEM (10% FBS) and incubated for 24 hours at 37° C., 5% CO2. Two hours before infection, the medium was replaced with 100 L of DMEM (2% FBS) containing the compound of interest at concentrations 50% greater than those indicated, including a DMSO control. Plates were then transferred into the BSL3 facility and 100 PFU (Vero-TMPRSS2 MOI=0.025) or 1000 PFU (HeLa-ACE2 MOI=0.25) of indicated variant was added in 50 L of DMEM (2% FBS), bringing the final compound concentration to those indicated. Plates were then incubated for 48 hours at 37° C. After infection, supernatants were removed and cells were fixed with 4% formaldehyde for 24 hours prior to being removed from the BSL3 facility. The cells were then immunostained for the viral NP protein (an mAb 1C7) with a DAPI counterstain. Infected cells (488 nm) and total cells (DAPI) were quantified using the Celigo (Nexcelcom) imaging cytometer. Infectivity was measured by the accumulation of viral N protein (fluorescence accumulation). Percent infection was quantified as ((Infected cells/Total cells)−Background)*100 and the DMSO control was then set to 100% infection for analysis. Data was fit using nonlinear regression and IC50s for each experiment were determined using GraphPad Prism version 8.0.2 (San Diego, CA). Cytotoxicity was also performed using the MTT assay (Roche), according to the manufacturer's instructions. Cytotoxicity was performed in uninfected Vero-TMPRSS2 or HeLa-ACE2 cells with same compound dilutions and concurrent with viral replication assay. All assays were performed in biologically independent triplicates.

Method 2. SARS-CoV-2 hCOV-229E/hCOV-OC43

### Reduction of Virus-Induced Cytopathic Effect (Primary CPE Assay)

Confluent or near-confluent cell culture monolayers of Vero E6 cells are prepared in 96-well disposable microplates the day before testing. Cells are maintained in MEM supplemented with 5% FBS. For antiviral assays the same medium is used but with FBS reduced to 2% and supplemented with 50-μg/ml gentamicin. Compounds are dissolved in DMSO, saline or the diluent requested by the submitter. Less soluble compounds are vortexed, heated, and sonicated, and if they still do not go into solution are tested as colloidal suspensions. The test compound is prepared at four serial log 10 concentrations, usually 0.1, 1.0, 10, and 100 μg/ml or μM (per sponsor preference). Lower concentrations are used when insufficient compound is supplied. Five microwells are used per dilution: three for infected cultures and two for uninfected toxicity cultures. Controls for the experiment consist of six microwells that are infected and not treated (virus controls) and six that are untreated and uninfected (cell controls) on every plate. A known active drug is tested in parallel as a positive control drug using the same method as is applied for test compounds. The positive control is tested with every test run.

Growth media is removed from the cells and the test compound is applied in 0.1 ml volume to wells at 2× concentration. Virus, normally at ˜60 CCID50 (50% cell culture infectious dose) in 0.1 ml volume is added to the wells designated for virus infection. Medium devoid of virus is placed in toxicity control wells and cell control wells. Plates are incubated at 37° C. with 5% CO2 until marked CPE (>80% CPE for most virus strains) is observed in virus control wells. The plates are then stained with 0.011% neutral red for approximately two hours at 37° C. in a 5% CO2 incubator. The neutral red medium is removed by complete aspiration, and the cells may be rinsed 1× with phosphate buffered solution (PBS) to remove residual dye. The PBS is completely removed, and the incorporated neutral red is eluted with 50% Sorensen's citrate buffer/50% ethanol for at least 30 minutes. Neutral red dye penetrates into living cells, thus, the more intense the red color, the larger the number of viable cells present in the wells. The dye content in each well is quantified using a spectrophotometer at 540 nm wavelength. The dye content in each set of wells is converted to a percentage of dye present in untreated control wells using a Microsoft Excel computer-based spreadsheet and normalized based on the virus control. The 50% effective (EC50, virus-inhibitory) concentrations and 50% cytotoxic (CC50, cell-inhibitory) concentrations are then calculated by regression analysis. The quotient of CC50 divided by EC50 gives the selectivity index (SI) value. Compounds showing SI values >10 are considered active.

### Reduction of Virus Yield (Secondary VYR Assay)

Active compounds are further tested in a confirmatory assay. This assay is set up similar to the methodology described above only eight half-log 10 concentrations of inhibitor are tested for antiviral activity and cytotoxicity. After sufficient virus replication occurs (3 days for SARS-CoV-2), a sample of supernatant is taken from each infected well (three replicate wells are pooled) and tested immediately or held frozen at −80° C. for later virus titer determination. After maximum CPE is observed, the viable plates are stained with neutral red dye. The incorporated dye content is quantified as described above to generate the EC50 and CC50 values.

The VYR test is a direct determination of how much the test compound inhibits virus replication. Virus yielded in the presence of test compound is titrated and compared to virus titers from the untreated virus controls. Titration of the viral samples (collected as described in the paragraph above) is performed by endpoint dilution (Reed and Muench, “A Simple Method of Estimating Fifty Percent Endpoints.” Am J Hyg 27 (1938): 493-98). Serial 1/10 dilutions of virus are made and plated into 4 replicate wells containing fresh cell monolayers of Vero E6 cells. Plates are then incubated, and cells are scored for presence or absence of virus after distinct CPE is observed, and the CCID50 calculated using the Reed-Muench method (24). The 90% (one log 10) effective concentration (EC90) is calculated by regression analysis by plotting the log 10 of the inhibitor concentration versus log 10 of virus produced at each concentration. Dividing EC90 by the CC50 gives the SI value for this test.

Method 3. hCOV-229E

Inhibition of virus-induced cytopathic effects (CPE) and cell viability following alpha coronavirus 229E replication in MRC-5 cells was measured using the XTT tetrazolium dye. MRC-5 cells were maintained in complete culture medium consisting of Dulbecco's Modified Eagle Medium (DMEM) supplemented with 10% fetal bovine serum (FBS), 2 mM L-glutamine, 100 U/mL penicillin, and 100 μg/mL streptomycin. For the assay, cells (5×10³ per well) were seeded into 96-well flat-bottom tissue culture plates and allowed to adhere overnight. Following overnight incubation, the medium was replaced with assay medium composed of DMEM without phenol red, supplemented with 2% FBS, 2 mM L-glutamine, 100 U/mL penicillin, 100 μg/mL streptomycin, 1 mM sodium pyruvate, and 0.1 mM nonessential amino acids. Diluted test compounds and virus-pre-titrated to produce 85-95% cell killing by six days post-infection-were added to the wells. Plates were incubated for six days at 37° C. in a humidified atmosphere containing 5% CO₂. Cell viability was assessed by XTT staining, and optical density was measured at 450 and 650 nm using SoftMax Pro 5.4.2 software. Percent CPE reduction in virus-infected wells and percent cell viability in uninfected drug control wells were used to calculate EC₅₀ and TC₅₀ values via four-parameter curve fit analysis.

### Method 4. HCV Replicon

The reporter cell line Huh-luc/neo-ET was obtained from Dr. Ralf Bartenschlager (Department of Molecular Virology, Hygiene Institute, University of Heidelberg, Germany) by ImQuest BioSciences through a specific licensing agreement. This cell line harbors the persistently replicating I₃₅₉luc-ubi-neo/NS3-3′/ET replicon containing the firefly luciferase gene-ubiquitin-neomycin phosphotransferase fusion protein and EMCV IRES driven NS3-5B HCV coding sequences containing the ET tissue culture adaptive mutations (E1202G, T1208I, and K1846T). A stock culture of the Huh-luc/neo-ET was expanded by culture in DMEM supplemented with 10% FCS, 2 mM glutamine, penicillin (100 IU/mL)/streptomycin (100 μg/mL) and 1× nonessential amino acids plus 1 mg/mL G418. The cells were split 1:4 and cultured for two passages in the same media plus 250 μg/mL G418. The cells were treated with trypsin and enumerated by staining with trypan blue and seeded into 96-well tissue culture plates at a cell culture density 7.5×10³ cells per well and incubated at 37° C. 5% CO₂ for 24 hours.

### Method 5. HCV Replicon

The reporter cell line Huh-luc/neo-ET was obtained from Dr. Ralf Bartenschlager (Department of Molecular Virology, Hygiene Institute, University of Heidelberg, Germany) by ImQuest BioSciences through a specific licensing agreement. This cell line harbors the persistently replicating I₃₅₉luc-ubi-neo/NS3-3′/ET replicon containing the firefly luciferase gene-ubiquitin-neomycin phosphotransferase fusion protein and EMCV IRES driven NS3-5B HCV coding sequences containing the ET tissue culture adaptive mutations (E1202G, T1208I, and K1846T). A stock culture of the Huh-luc/neo-ET was expanded by culture in DMEM supplemented with 10% FCS, 2 mM glutamine, penicillin (100 IU/mL)/streptomycin (100 μg/mL) and 1× nonessential amino acids plus 1 mg/mL G418. The cells were split 1:4 and cultured for two passages in the same media plus 250 μg/mL G418. The cells were treated with trypsin and enumerated by staining with trypan blue and seeded into 96-well tissue culture plates at a cell culture density 7.5×10³ cells per well and incubated at 37° C. 5% CO₂ for 24 hours.

### Method 6. Norwalk Virus

Compounds were tested in an assay medium containing 2% fetal bovine serum (FBS). Test compounds were added to cells prior to infection with virus at a multiplicity of infection (MOI) sufficient to induce visible cytopathic effect (CPE). When maximal CPE was observed in virus control wells, cell viability was assessed using a neutral red dye-uptake assay. Resulting data were used to calculate the 50% effective concentration (EC₅₀; concentration required to reduce virus replication by 50%), the 50% cytotoxic concentration (CC₅₀; concentration that reduces cell viability by 50%), and the selective index (SI=CC₅₀ or CC₉₀/EC₅₀) for each compound. Additionally, the EC₉₀ was defined as the concentration required to achieve a 1 log₁₀ reduction in viral titer. Dose-response curves depicting viral CPE (red line) and compound cytotoxicity (purple line) were generated to support these analyses.

Method 7. Mt. Sinai, Influenza a Virus

A549 cells were infected with each viral strain at MOI 0.05. After 24 h post-infection with A/WSN/33 cells were fixed with 4% formaldehyde for 30 min. Cells were briefly washed with PBS, then permeabilized with 0.1% Triton X-100 in PBS for 15 minutes. Blocking occurred at room temperature for 1 hour with 0.5% BSA in PBS followed by incubation with the NP antibody (HT103, a gift from Thomas Moran) in 0.5% BSA in PBS for 1 hour at room temperature. Cells were washed with PBS 2× and incubated with a fluorescently-labeled secondary antibody, alexa-fluor-488 (Invitrogen), in 0.5% BSA in PBS with DAPI for 45 min at room temperature. Two washes with PBS were performed before imaging the cells on a Celigo Image Cytometer. Infected cells (488 nm) and total cells (DAPI) were quantified using the Celigo (Nexcelcom) imaging cytometer. Infectivity was measured by the accumulation of viral NP protein (fluorescence accumulation). Percent infection was quantified as ((Infected cells/Total cells)−Background)*100 and the DMSO control was then set to 100% infection for analysis. Data was fit using nonlinear regression and IC50s for each experiment were determined using GraphPad Prism version 8.0.2 (San Diego, CA). Cytotoxicity was also performed using the MTT assay (Roche), according to the manufacturer's instructions. Cytotoxicity was performed in uninfected A549 cells with same compound dilutions and concurrent with viral replication assay. All assays were performed in biologically independent triplicates.

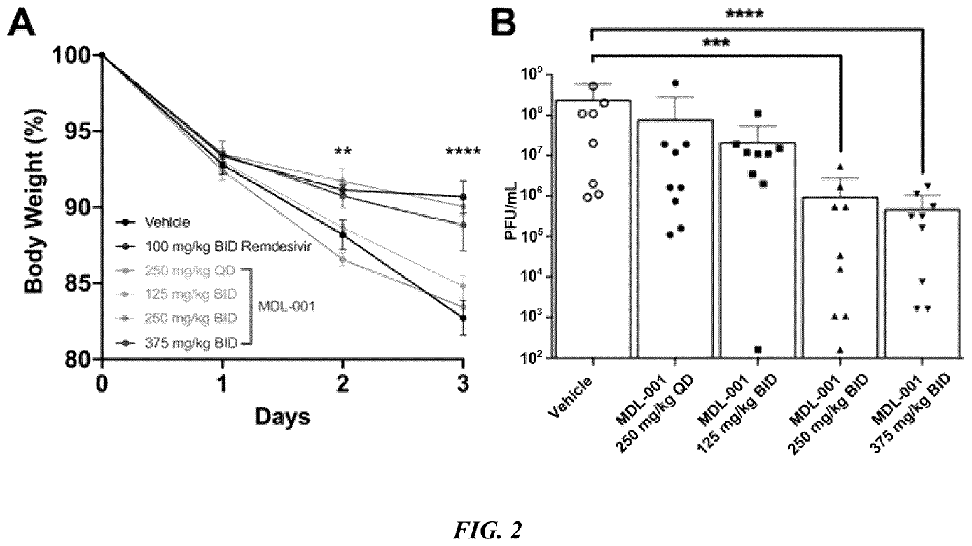

In vivo antiviral efficacy of pipendoxifene in mice infected with a mouse-adapted (MA) viral variant, designated as MA-SARS-CoV-2, was investigated. As shown in FIG. 2, pipendoxifene prevented body weight loss and viral accumulation in the lung in a dose-dependent manner. The reduction in body weight loss by pipendoxifene compared to the vehicle became statistically significant on days 2 and 3. Furthermore, the two highest pipendoxifene doses (250 and 375 mg/kg BID) resulted in a body weight loss reduction similar in magnitude to the remdesivir control arm. Noticeably, the decrease in viral lung titers upon treatment with pipendoxifene mirrored the dose-dependent pattern observed for weight loss, supporting the notion that a reduction in weight loss provoked by viral infection was correlated to virus clearance.

In vivo efficacy of pipendoxifene was evaluated using a mouse-adapted (MA) SARS-CoV variant to simulate human disease in mice. The goal was to assess its therapeutic potential in a preclinical setting. The readout values for two features were quantitated: reduction in body weight loss, as a surrogate of symptomatic relief, and viral load. Data was analyzed by two-way ANOVA (*P<0.05, **P<0.01, ***P<0.001, and ****P<0.0001). The symptomatic relief method tracks changes in body weight as an indicator of disease progression and treatment efficacy. The dose range tested included 125 mg/kg BID, 250 mg/kg QD, 250 mg/kg BID, and 375 mg/kg BID. The viral yield reduction assays determine viral titers and were quantified to measure the reduction in viral load following treatment with pipendoxifene.

### Example 2

### Overview of the Experimental Workflow

The examples presented herein describe a broad-spectrum antiviral development approach using a recently developed GALILEO, an artificial intelligence (AI) drug discovery suite featuring ChemPrint—a graph convolutional network, which learns from geometric graph embeddings. The new chemical entities (NCEs) designed using the AI drug discovery suite were further verified with downstream in vitro assays.

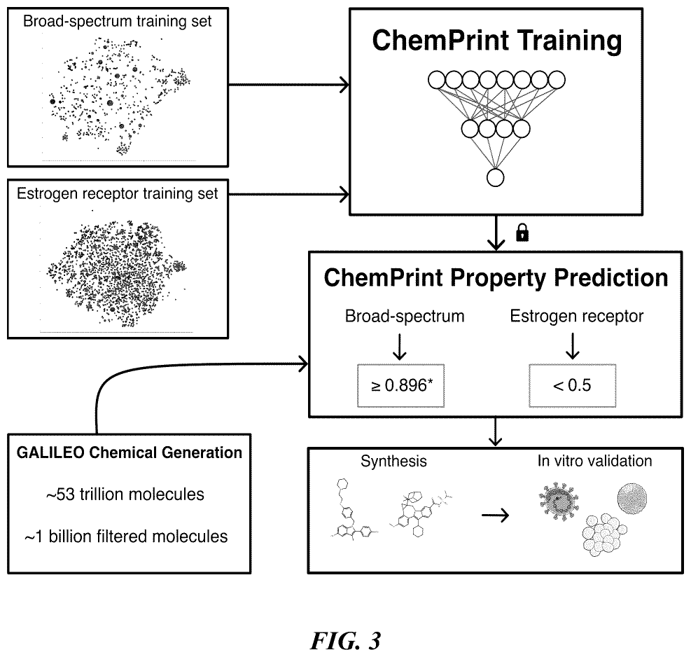

FIG. 3 depicts a schematic of the experimental workflow used herein, demonstrating the generative and multimodal drug discovery approach to remove off-target interactions while optimizing antiviral properties.

The AI drug discovery suite used herein and related methods are described in Umansky, T. et al. (bioRXiv 2024.03.22.586314 (2024) doi:10.1101/2024.03.22.586314), Woods, V., et al. (bioRXiv 2024.03.29.587401 (2024) doi:10.1101/2024.03.29.587401), and Woods, V. et al. (bioRXiv (2025) doi:10.1101/2025.01.13.632836), the contents of which are incorporated herein by reference in their entireties. Leveraging GALILEO's bioinformatics tools and ChemPrint, it was discovered that the well-investigated Thumb-1 pocket in the RNA-dependent RNA polymerase (RdRp) of Hepatitis C Virus (HCV) is structurally conserved in many pathogenic viruses, making it a druggable target for broad-spectrum antiviral compounds. pipendoxifene (with demonstrated safety in rodents and humans at high concentrations for extended durations) was also discovered as the first molecule within this novel class of Thumb-1 broad-spectrum antiviral inhibitors, a class that had not been recognized before.

Advancing AI-driven drug discovery requires (1) designing therapeutics for novel targets with little to no training data (one-shot learning); (2) generating ultra-large and relevant in silico chemical screening libraries; (3) discovering multiparameter-optimized NCEs; and (4) achieving high in vitro hit rates with minimal screening. In the present study, the examples described herein set out to validate such a framework by integrating generative, one-shot, multimodal, and predictive modeling to efficiently navigate vast chemical spaces and optimize multiple molecular properties at the same time. The present study build on the success of pipendoxifene, a broad-spectrum antiviral compound with exceptional efficacy and safety, by leveraging the GALILEO platform to create a library of next generation, broad-spectrum antiviral NCEs.

Specifically, the method used herein: (1) leveraged pipendoxifene, the only known broad-spectrum antiviral Thumb-1 inhibitor, as training data in a one-shot learning approach to discover NCEs within this same novel class using ChemPrint; (2) generated 53 trillion NCEs across a large chemical space, starting from pipendoxifene's pharmacophoric scaffold; (3) discovered 15 multiparameter-optimized NCEs through a multimodal approach, based on broad-spectrum antiviral activity, elimination of off-target binding, and chemical diversity; and (4) achieved a 100% in vitro hit rate.

A one-shot prediction refers to ChemPrint's ability to identify NCEs as a novel class of antivirals, with pipendoxifene as the only antiviral of its class in the training set, without requiring iterative feedback or re-training. When validated in vitro, all 15 NCEs provided in Table 1 exhibited antiviral activity (100% hit rate).

This study highlights the power of the one-shot, multimodal approach to systematically discover large, optimized NCE libraries containing potent compounds for complex targets with off-target interactions eliminated at high hit-rates. The study also represents a significant milestone in a multi-generational therapeutic program that began with pipendoxifene and continues to drive advancements in NCE discovery and optimization.

The workflow presented here underlines the advantages of generative tools like GALILEO, which by creating large chemical spaces, open the door to on-demand drug development programs with a high hit rate and designed target specificity. ChemPrint also brings great value, enabling the one-shot identification of drugs, such as the 15 NCEs provided in Table 1, that are optimized for multiple properties, through a multimodal approach.

### Example 3

### General Methods

This example describes the general methods, materials, and protocols used in Examples 4-7 below.

### Computational Prediction

### Broad-Spectrum Training Data

The ChemPrint dataset comprises a set of small molecules with well-documented antiviral activity. This dataset included small molecules active against four viral families: Coronaviridae, Picornaviridae, Flaviviridae, and Togaviridae. Antiviral compounds were labeled as “explicit” if their Thumb-1 site inhibition in HCV's RdRp was directly identified in the literature. Compounds were labeled as “implicit” if they targeted the RdRp of any ssRNA virus without direct evidence of Thumb-1 binding but matched the Thumb-1 pharmacophoric profile or contained core scaffolds consistent with Thumb-1 inhibitors. Both explicit and implicit inhibitors were included in the broad-spectrum training dataset.

### Broad-Spectrum Training Data Processing

Small molecules were removed from the dataset if their size fell outside a 180-800 Da size range, if they lacked Kᵢ, K_{d}, IC₅₀ or EC₅₀ values, or if they contained a PAINS (Pan-Assay Interference Compounds) substructure, a category of molecules documented for their non-specific activity. Small molecules with an ‘indol_3yl_alk(461)’ substructure were kept because this moiety is commonly found in antiviral drugs. A binary classification dataset was then created by defining a line of best fit across viral datasets to establish a relationship between negative log bioactivity measures. Missing values were imputed using this relationship, and an orthogonal line to the best fit determined the activity cutoff. Compounds were classified as active if they inhibited two or more viruses or, in cases of limited testing, a single virus. This process resulted in 1,282 compounds, with 602 classified as active and 680 as inactive.

### Estrogen Receptor Training Data

ChemPrint was used to identify and remove NCEs predicted to interact with the human estrogen receptor. The dataset comprised small molecule ligands known to bind ERα (Uniprot ID: P03372) and ERβ (Uniprot ID: Q92731). Bioactivity data for these ligands were retrieved from PubChem, BindingDB, and ChEMBL, including compounds classified as agonists, antagonists, or selective estrogen receptor modulators (SERMs). Using data from all these categories was appropriate because they share the common feature of binding to the estrogen receptor, enabling comprehensive identification of compounds with potential receptor interaction.

### Estrogen Receptor Training Data Processing

As was done with the Thumb-1 training dataset, small molecules were removed if their size fell outside the 180-800 Da range, if they lacked Kᵢ, K_{d}, IC₅₀ or EC₅₀ values, or if they contained a PAINS (Pan-Assay Interference Compounds) substructure, excluding ‘indol_3yl_alk(461)’. Duplicates were resolved by retaining the most potent readout, irrespective of the type of bioactivity measurement or ERα/ERβ interaction. An activity cutoff of ˜75 nM was applied to classify compounds as active or inactive, creating a balanced dataset. This threshold was selected to identify compounds with lower potency relative to pipendoxifene, which has an IC50 of 14 nM against ERα. The final dataset included 4,208 compounds, with 2,103 classified as active and 2,105 as inactive.

### ChemPrint Model Architecture

ChemPrint architecture leverages Mol-GDL to learn the adaptive embeddings derived from its training data to make predictions. The input data takes the form of a geometric graph, a molecular representation that encapsulates the structural information of each datapoint, to which one-hot encoded features are passed in. ChemPrint architecture encompasses an end-to-end Graph Convolutional Network (GCN) with a Multilayer Perceptron (MLP) module to facilitate positive and negative classification. Select normalization, activation, pooling, and dropout layers were used.

### Model Training for Thumb-1 Interactors

Datasets were split 50/50 into training and validation sets, with the t-SNE split methodology. Receiver operating characteristic curves (AUROC) to assess the model's performance during hyperparameter optimization (FIG. 6). For large-scale inference, a single broad-spectrum model optimized with the full dataset was deployed.

### Model Training for Estrogen Receptor Interactors

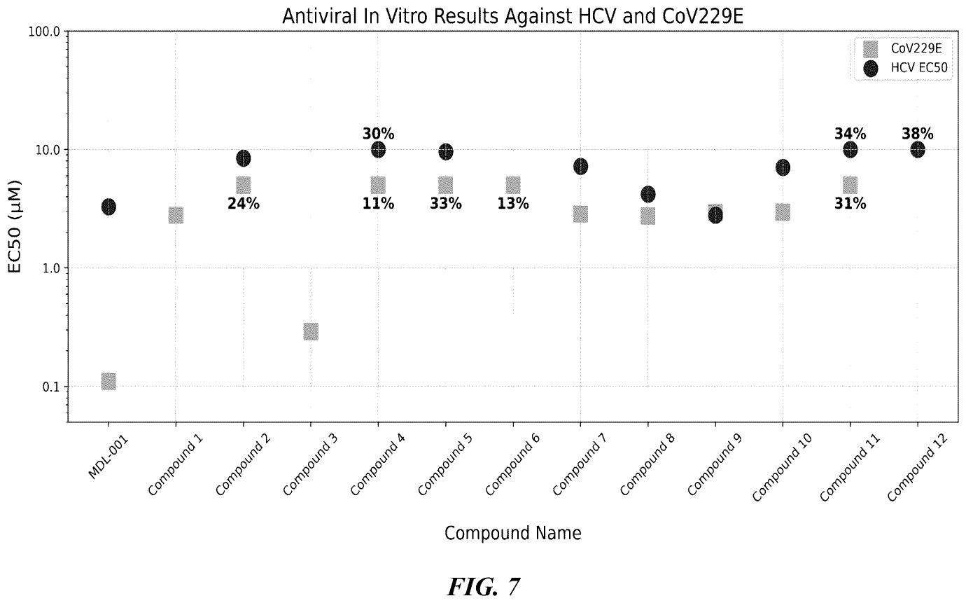

Datasets were split and model performance were achieved as described in the above subsection for the model trained to predict Thumb-1 interactors (FIG. 7). In this case inference was accomplished using an ensemble of 10 estrogen receptor ChemPrint models, retrained on the full dataset.

### Broad-Spectrum Combinatorial Space

The combinatorial chemical space was generated from pipendoxifene's pharmacophoric scaffold and amounted to 53 trillion unique small molecules. Each of these small molecules was decorated with up to five R group substituents, designed to engage in hydrogen bond interactions with the Thumb-1 pocket. The scaffolds and substituent positions were informed by training data targeting the Thumb-1 site and synthesizability.

### Broad-Spectrum Inference Space

The broad-spectrum inference library was generated by reducing the combinatorial space to approximately one billion compounds. This was achieved by removing stereospecificity from all substituents (R₁—R5) and simplifying substituent structures based on synthetic accessibility. Adjustments included the removal of certain alkyl groups and reducing the complexity of R group combinations. These steps allowed for a more manageable and focused prediction library, while retaining chemistry representative of the entire combinatorial space.

### Model Inference for Thumb-1 Interactors

The broad-spectrum Thumb-1 ChemPrint model was deployed for brute force inference on approximately one billion compounds. Data featurization and inference required ˜2,000 computational hours, utilizing multiprocessing on a system with 48 CPU cores. By leveraging 100 CPUs, each with 48 cores operating in parallel, the screening of one billion compounds was completed in under 24 hours.

### Model Inference for Estrogen Receptor Interactors

An ensemble of 10 estrogen receptor ChemPrint models was applied to the refined list of 500 NCEs filtered by the broad-spectrum Thumb-1 ChemPrint model. This inference step was performed to identify and exclude compounds predicted to interact with the estrogen receptor.

### Chemical Space Data

The datasets used for chemical space analysis comprised small molecules with well-documented antiviral activity. These included compounds active against the viral families Coronaviridae, Picornaviridae, Flaviviridae, and Togaviridae. Compounds were classified as ‘explicit’ inhibitors if their activity was directly linked to the Thumb-1 pocket in HCV's RdRp. Compounds were classified as ‘implicit’ inhibitors if they targeted viruses without direct evidence of Thumb-1 binding but matched the Thumb-1 pharmacophoric profile or contained core scaffolds consistent with Thumb-1 inhibitors. Additional data included nucleoside/nucleotide inhibitors (active site inhibitors), other allosteric RdRp inhibitors (not targeting Thumb-1), pipendoxifene, Beclabuvir, a subset of ChemPrint-predicted broad-spectrum Thumb-1 active compounds, and the 12 NCEs (Compounds 1-12 provided in Table 1).

Chemical Space Mapping with PCA and t-SNE

Principal component analysis (PCA) and t-distributed stochastic neighbor embedding (t-SNE) were used to visualize chemical space relationships. Both methods used chemical structures as input features, represented as ECFP4 fingerprints. No mechanism of action (MoA) labels were used as input features for the analysis. PCA, a linear dimensionality reduction technique, captured global relationships in the dataset, with the distance between points reflecting structural similarity. In contrast, t-SNE, a non-linear technique, better preserved local relationships, providing insight into clustering within the chemical space. The results of PCA and t-SNE were visualized in two-dimensional plots to compare the relative positioning of pipendoxifene, Beclabuvir, the 15 NCEs, ChemPrint-predicted compounds, and curated antiviral datasets.

### Tanimoto Similarity Analysis

Tanimoto coefficients were calculated to quantify chemical similarity between compounds. ECFP4 fingerprints were used to compute pairwise similarity scores, ranging from 0 to 1. Two compounds were considered chemically similar if the Tanimoto coefficient was ≥0.5, a threshold commonly used in the industry to denote significant similarity. Comparisons were performed between the 15 NCEs provided in Table 1, pipendoxifene, and Beclabuvir to assess their relative novelty and diversity within the chemical space

### Compound Synthesis

The 15 NCEs provided in Table 1 were synthesized at Piramal Pharma Solutions. Purity was confirmed to be greater than 95% for all compounds, verified through liquid chromatography-mass spectrometry (LC-MS), high-performance liquid chromatography (HPLC), and proton nuclear magnetic resonance (¹H-NMR) spectroscopy. These methods ensured the chemical integrity and suitability of the synthesized compounds for subsequent testing and analysis.

### In Vitro Antiviral Assays

### Cell Culture

The Huh-lucineo-ET reporter cell line was obtained from Dr. Ralf Bartenschlager (Department of Molecular Virology, Hygiene Institute, University of Heidelberg, Germany) by ImQuest BioSciences through a specific licensing agreement. This cell line harbors the persistently replicating 1389/NS3-37LucUbiNeo-ET replicon of HCV genotype 1b containing the firefly luciferase gene-ubiquitin-neomycin phosphotransferase fusion protein and EMCV IRES driven N53-5B HCV coding sequences containing the ET tissue culture adaptive mutations (E1202G, T12081, and K1846T). For the first two passages after thawing, the cells were cultured in DMEM without phenol red (Gibco, catalog no. 31053028) supplemented with fetal bovine serum (FBS) (Gibco, catalog no. 10082147), 2 mM GlutaMAX Supplement (Gibco, catalog no. 35050061), 100 U/mL penicillin and 100 μg/mL streptomycin (Corning, catalog no. 30-002-CI), and 1× nonessential amino acids (Gibco, catalog no. 11140050) plus 1 mg/mL G418 (Gibco, catalog no. 10131035). The cells were then further cultured in the complete medium described above plus 500 μg/mL G418. One day prior to test article addition, the cells were seeded at a density of 5×103 cells per well in 100 μL complete medium plus 500 μg/mL G418 in either standard clear or white-walled/clear-bottom 96-well tissue culture plates for colorimetric or chemiluminescent endpoint assays, respectively. The cells were incubated at 37° C./5% CO2 for 24 hours.

### Huh-Lucineo-ET Treatment Conditions

Twenty-four hours after seeding the cells, the cell culture supernatant was removed from each well and replaced with 175 μL fresh complete medium minus G418. Test compounds were evaluated at a high-test concentration of 10 μM and five serial half-logarithmic dilutions in three biological replicates. In three biological replicates, Sofosbuvir was evaluated in parallel at a high-test concentration of 1 μM and five serial half-logarithmic dilutions. Compounds were first serially diluted in DMSO to 400× of each final in-well concentration. An intermediate 8× solution was prepared in a complete medium and then added to the cells in a volume of 25 μL per well. A complete medium containing an equal percentage of DMSO (v/v) was added to vehicle-treated control cells. The final DMSO concentration was 0.25% (v/v) for all samples. The cells were incubated for an additional 72 hours at 37° C./5% CO2, then HCV replication was measured by luciferase activity and cell viability was assessed in parallel using an XTT colorimetric assay.

### Measurement of HCV Replication

HCV replication from the replicon assay system was measured following 72 hours of incubation using the britelite plus Reporter Gene Assay according to the manufacturer's protocol (Revvity, catalog no. 6066761). In brief, one vial of britelite plus substrate was solubilized in 10 mL of britelite reconstitution buffer, mixed gently by inversion, and incubated for five minutes at room temperature. One hundred microliters (100 μL) of cell culture supernatant were removed from each well, followed by adding 100 μL britelite plus reagent and thorough mixing by pipetting up and down. The plates were sealed with adhesive film and incubated at room temperature for 10 minutes. Chemiluminescence was measured as relative light units on a FlexStation 3 microplate reader (Molecular Devices). The raw data were imported into Microsoft Excel for further analysis. Data are presented as relative light units (RLU) or percent relative to vehicle-treated, virus-only control (mean+1—standard deviation, n=3 biological replicates from one independent experiment).

### XTT Cell Viability Assay

Huh-luc/neo-ET cell viability was measured at 72 hours after treatment using the tetrazolium dye XTT (2,3-bis(2-methoxy-4-nitro-5-sulfophenyI)-5[(phenylamino)carbonyl]-2H-tetrazolium hydroxide). Mitochondrial enzymes of metabolically active cells metabolize XTT-tetrazolium to a soluble formazan product. XTT solution was prepared fresh at 1 mg/mL in DMEM. Phenazine methosulfate (PMS) solution was prepared at 0.15 mg/mL in PBS and stored in the dark at −20° C. An XTT/PMS stock was prepared immediately before use by adding 40 μL of PMS per ml of XTT solution. Fifty microliters (50 μL) XTT/PMS were added per well, and the cells were incubated for 4 hours at 37° C. The cell culture plates were then sealed with adhesive plate sealers and shaken gently or inverted several times to mix the soluble formazan product. The formazan product was measured spectrophotometrically by absorbance at 450/650 nm on a Vmax plate reader (Molecular Devices). The raw data in Softmax Pro 5.4.2 software was imported into Microsoft Excel for further analysis. Data are presented as absorbance values (450/650 nm) or the percentage of viable cells relative to vehicle-treated control (mean+1—standard deviation, n=3 biological replicates from one independent experiment).

### In Vitro Activity and Anti-Coronavirus 229E Cytoprotection Assay

### Cell Preparation

MRC-5 cells obtained from ATCC (CCL-171) were passaged in DMEM supplemented with 10% FBS, 2 mM L-glutamine, 100 U/mL penicillin, 100 μg/mL streptomycin, 1 mM sodium pyruvate, and 0.1 mM NEAA in T-75 flasks prior to use in the antiviral assay. On the day preceding the assay, the cells were split 1:2 to ensure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 3×103 cells per well in a tissue culture medium and added to flat bottom microtiter plates in a volume of 100 μL. The plates were incubated at 37° C./5% CO2 overnight to allow for cell adherence.

### Virus Preparation

Coronavirus229E (CoV229E) was obtained from ATCC (VR-740) and grown in MRC-5 cells to produce a stock virus pool. A pretittered aliquot of the virus was removed from the freezer (−80° C.) and allowed to thaw slowly to room temperature in a biological safety cabinet. The virus was resuspended and diluted into assay medium (DMEM supplemented with 2% heat-inactivated FBS, 2 mM L-glutamine, 100 U/mL penicillin, 100 μg/mL streptomycin) such that the amount of virus added to each well in a volume of 100 μL was the amount determined to yield 85 to 95% cell killing at 6 days post-infection.

### Plate Format

Each plate contains cell control wells (cells only), virus control wells (cells plus virus), triplicate drug toxicity wells per compound (cells plus drug only), and triplicate experimental wells (drug plus cells plus virus).

### Efficacy and Toxicity XTT

Following incubation at 37° C. in a 5% CO2 incubator for six days, the test plates were stained with the tetrazolium dye XTT (2,3-bis(2-methoxy-4-nitro-5-sulfophenyl)-5-[(phenylamino)carbonyl]-2H-tetrazolium hydroxide). The mitochondria enzymes of metabolically active cells metabolized XTT-tetrazolium to a soluble formazan product, allowing rapid quantitative analysis of the inhibition of virus-induced cell killing by antiviral test substances. XTT solution was prepared daily as a 1 mg/mL stock in RPMI1640. Phenazine methosulfate (PMS) solution was prepared at 0.15 mg/mL in PBS and stored in the dark at −20° C. XTT/PMS stock was prepared immediately before use by adding 40 μL of PMS per ml of XTT solution. Fifty microliters of XTT/PMS was added to each well of the plate, and the plate was reincubated for 4 hours at 37° C. Plates were sealed with adhesive plate sealers and shaken gently or inverted several times to mix the soluble formazan product. The plate was read spectrophotometrically at 450/650 nm with a Molecular Devices Vmax plate reader.

### MCF7 Growth Inhibition Assays

### Assay Conditions

The growth inhibition properties of inhibitors on MCF7 wild-type cell lines were evaluated using the Cell Titre Glo 2.0 cell viability reagent. The assay utilized 384-well black clear bottom plates with 20 μl of MCF7 wild-type cell suspension mixed with reference/test compounds and 20 μl of Cell Titre Glo 2.0 reagent. Cells were seeded at a density of 1000 cells/well in DMEM High Glucose culture media+10% Fetal Bovine Serum (FBS). Staurosporine and Camptothecin served as positive controls for confirming cytotoxicity at a concentration of 30 μM, and vehicle control of 1% DMSO was used to standardize conditions across all test points. The assay and culture media both contained 1% DMSO. Assays were conducted in duplicate for each concentration of the test compounds. The incubation period for the assay was set at 120 hours, after which growth inhibition was assessed. Using a multi-mode reader, the assay readout was based on luminescence measured in Relative Light Units (RLUs). The viability luminescence signal was monitored continually from the same wells over an extended period, allowing for detailed analysis of cell viability. Measurements were taken using an integration time ranging from 0.25 to 1 second across plates designated for various time points.

### Assay Protocol

Cells were maintained and seeded in 384 healthy plates, maintaining 1000 cells/well. Seeded plates were incubated at 37° C., 5% CO₂ for 16-18 hours to ensure optimal cell adherence and growth. Post-incubation, reference, and test compounds were added carefully to the wells for further processing. Plates were incubated again at 37° C., 5% CO₂ for 120 hours (5 days) to allow compound effects. This was followed by the addition of CTG reagent as per the kit's instruction protocol for 30 minutes and RLU data to be recorded. Post plate read data to be analyzed using GraphPad Prism for software IC₅₀ value generation.

### Example 4

### Generative Chemical Space

GALILEO generated a combinatorial chemical space of 53 trillion probable NCE antiviral compounds, by adding a combinatorial set of R group substituents to pharmacophoric scaffolds, inspired from pipendoxifene's chemical structure. The combinatorial chemical space was collapsed by reducing R substituent chemical complexity, resulting in an inference library of 1 billion compounds (see Example 2). The inference library containing 1 billion molecules retained NCE diversity features unique to the combinatorial chemical space, making the downstream computational tasks more manageable.

The inference library was processed using two ChemPrint models, trained to identify NCEs with a Thumb-1 binding probability higher than that of pipendoxifene (0.896); and to remove unspecific estrogen receptor binders, pipendoxifene's originally developed MoA (FIG. 3). ChemPrint is a convolutional neural network that extracts features from small molecules embedded as geometrical graphs. Thus, the NCEs were embedded in the inference library as geometrical graphs, and once embedded, the inference library dataset was loaded to ChemPrint. In the first case, the ChemPrint model trained to predict NCE/Thumb-1 interactions (see Example 2) was used. The results from this analysis revealed that from the 1-billion inference library input, four million NCEs had desired Thumb-1 binding probability. To assess the fidelity of ChemPrint at different positive probability thresholds, select promising compounds were retained. This list was narrowed down to 500 structurally diverse NCEs, after removing compounds with challenging synthetic pathways and halogen-reactive moieties.

Pipendoxifene (the pharmacophoric scaffold precursor of the combinatorial chemical space) is a known modulator of the estrogen receptor, raising the possibility that the NCEs may also modulate the estrogen receptor. Following the same approach used to develop ChemPrint for identifying NCEs with the desired property of broad-spectrum Thumb-1 interaction, the model was trained to exclude compounds with undesired properties, specifically estrogen receptor interaction (see Example 2). An estrogen receptor binding probability <0.5 was used to refine the 500-NCE list to a final shortlist of 12 NCEs (Compounds 1-12 provided in Table 1) (Table 4).

TABLE 4
Broad-Spectrum and Estrogen Receptor Binding ChemPrint model
predictions of 12 compounds nominated for synthesis.

| | Broad Spectrum | Estrogen Receptor |
|---|---|---|
| Compound ID | Probability | Binding Probability |
| Compound 1 | 0.9390 | 0.4664 |
| Compound 2 | 0.9345 | 0.4624 |
| Compound 3 | 0.9009 | 0.2707 |
| Compound 4 | 0.8727 | 0.2568 |
| Compound 5 | 0.9474 | 0.1872 |
| Compound 6 | 0.9453 | 0.1712 |
| Compound 7 | 0.6641 | 0.2584 |
| Compound 8 | 0.5614 | 0.2430 |
| Compound 9 | 0.9408 | 0.1713 |
| Compound 10 | 0.8636 | 0.2349 |
| Compound 11 | 0.9375 | 0.1560 |
| Compound 12 | 0.8280 | 0.2194 |

### Example 5

### Chemical Space Analysis

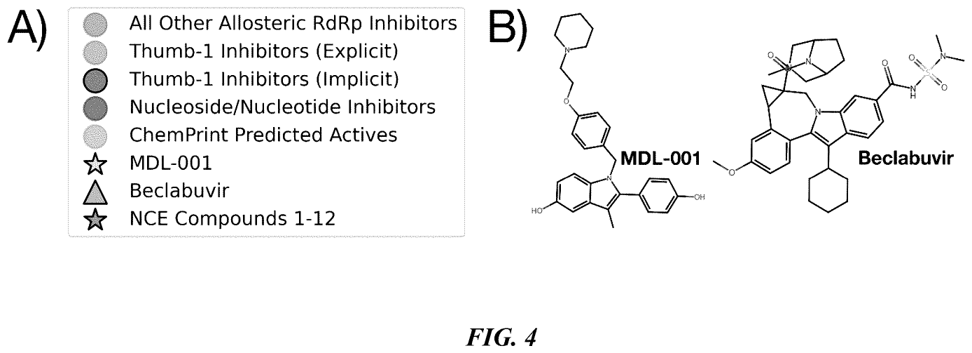

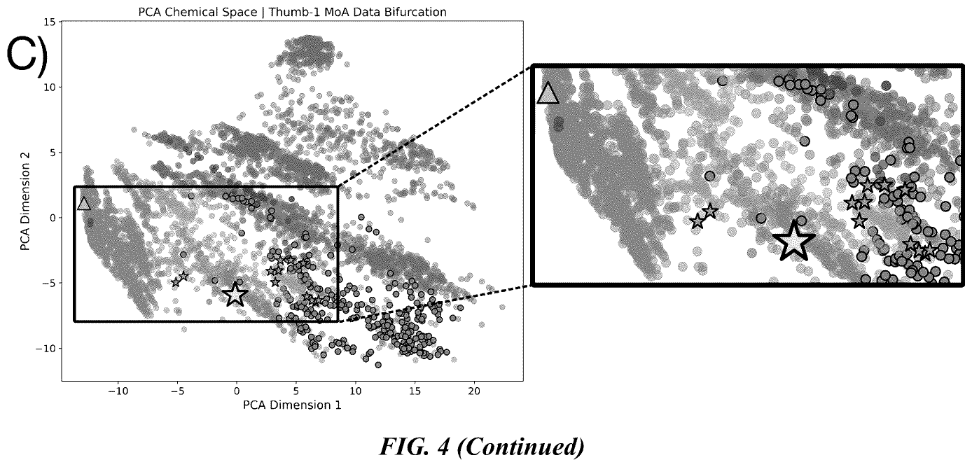

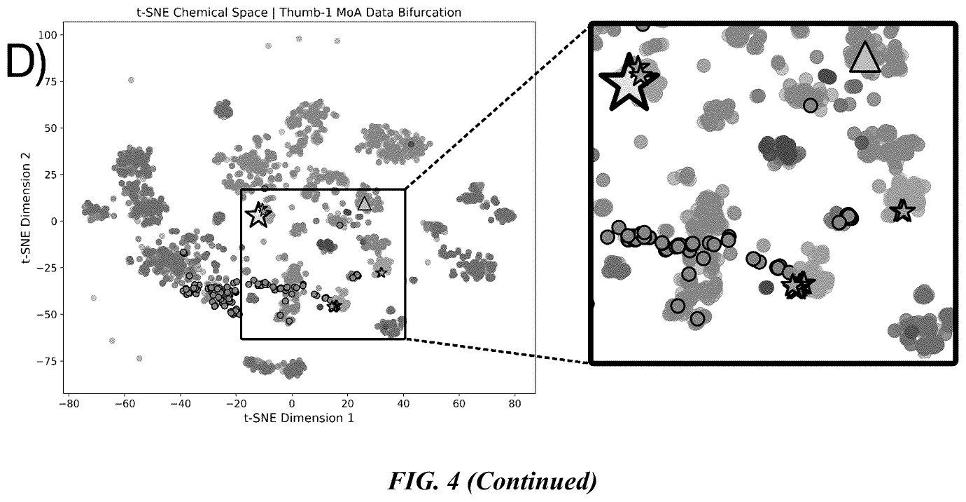

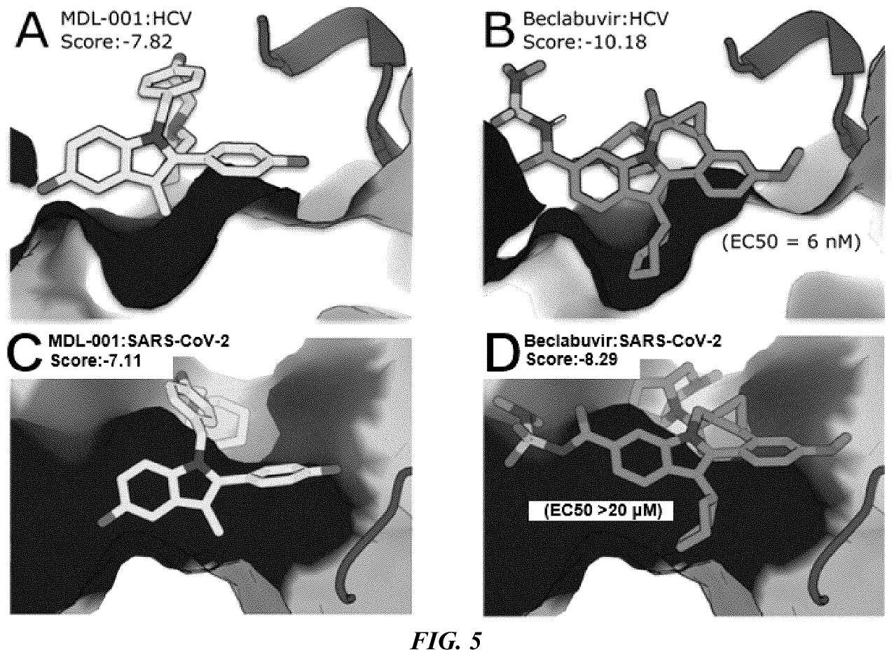

To evaluate the mechanisms of action (MoA), novelty, diversity, and similarity of the nominated NCEs, their chemical space were analyzed. Chemical space mapping was performed using principal component analysis (PCA) and t-distributed stochastic neighborhood embedding (t-SNE) to visualize relationships and Tanimoto coefficients to quantify them. The analysis included pipendoxifene, Beclabuvir, the 15 NCEs provided in Table 1, a subset of ChemPrint-predicted broad-spectrum Thumb-1 compounds, and curated antiviral datasets with known viral RdRp MoAs (labels explained in Example 2) (FIGS. 4A-B). Both PCA and t-SNE used only embedded chemical structures as input features, without incorporating MoA labels.

PCA (FIG. 4C) revealed a significant pattern of chemical distribution associated with documented MoAs. Pipendoxifene and the ChemPrint-predicted compounds, including 12 NCEs (Compounds 1-12 provided in Table 1), occupy a chemical space between ‘explicit’ inhibitors, known for targeting HCV's RdRp Thumb-1 pocket, and ‘implicit’ inhibitors, which target broader viral families without direct Thumb-1 evidence but align with its pharmacophoric profile or core scaffolds. Beclabuvir, in contrast, occupies a distinct corner of chemical space only occupied by ‘explicit’ HCV Thumb-1 inhibitors.

Some findings included: First, the PCA and t-SNE projections separate pipendoxifene from compounds targeting other sites, indicating a distinct mechanism. Second, pipendoxifene is positioned near “explicit” Thumb-1 inhibitors in the PCA, specifically those inhibiting HCV RdRp, and close to “implicit” Thumb-1 inhibitors in non-HCV viruses. Third, in the t-SNE projection, pipendoxifene occupies a unique position between HCV-only Thumb-1 inhibitors and non-HCV Thumb-1 inhibitors, suggesting its broad-spectrum potential. Fourth, beclabuvir, the only approved HCV Thumb-1 inhibitor, is chemically dissimilar to pipendoxifene, as evidenced by its distant positioning in both projections and Tanimoto similarity to pipendoxifene of 0.15, further emphasizing the novel nature of pipendoxifene as a broad-spectrum Thumb-1 inhibitor.

t-SNE (FIG. 4D), which better preserves local relationships, revealed clustering consistent with labeled MoAs. Three distinct local clusters containing the ChemPrint-predicted compounds, including 12 NCEs (Compounds 1-12 provided in Table 1), occupy the space near ‘explicit’ and ‘implicit’ inhibitors. One of these three clusters includes pipendoxifene, while none include Beclabuvir.

Tanimoto coefficients provided quantitative evidence of chemical similarity between 12 NCEs (Compounds 1-12 provided in Table 1) vs. pipendoxifene and Beclabuvir (FIG. 6). Values range from 0 to 1, with a Tanimoto coefficient of ≥0.5 (using ECFP4) indicating chemical similarity between two small molecules. 15 NCEs (provided in Table 1) received Tanimoto coefficients ranging from 0.28-0.47 compared to pipendoxifene, with an average similarity of 0.38, indicating chemical dissimilarity. When compared to Beclabuvir, the Tanimoto coefficients were even lower, ranging from 0.10-0.20, with an average similarity of 0.13. All NCE compounds reported scores ≤0.47 compared to pipendoxifene and 0.20 compared to Beclabuvir, demonstrating significant chemical novelty (Table 5).

TABLE 5
Tanimoto similarity scores of 15 NCEs

| Compound | Tanimoto to | Tanimoto to |
|---|---|---|
| Name | Pipendoxifene using | Beclabuvir using |
| (MCAA) | ECFP4 2048 bits | ECFP4 2048 bits |
| MCAA-01 | 0.48 | 0.20 |
| MCAA-02 | 0.47 | 0.20 |
| MCAA-03 | 0.36 | 0.20 |
| MCAA-04 | 0.72 | 0.17 |
| MCAA-05 | 0.33 | 0.23 |
| MCAA-06 | 0.46 | 0.12 |
| MCAA-07 | 0.31 | 0.14 |
| MCAA-08 | 0.45 | 0.10 |
| MCAA-09 | 0.30 | 0.12 |
| MCAA-10 | 0.45 | 0.10 |
| MCAA-11 | 0.30 | 0.12 |
| MCAA-12 | 0.42 | 0.10 |
| MCAA-13 | 0.42 | 0.10 |
| MCAA-14 | 0.28 | 0.12 |
| MCAA-15 | 0.28 | 0.12 |

While ChemPrint scored pipendoxifene with a high probability of successfully inhibiting the Thumb-1 site of RdRp, comparative docking (Autodock4) indicated that pipendoxifene had higher (worse) docking scores in both HCV and SARS-CoV-2 Thumb-1 sites than did beclabuvir. Specifically, pipendoxifene showed consistent docking scores of −7.82 kcal/mol and −7.11 kcal/mol for HCV and SARS-CoV-2, respectively. Pipendoxifene docked to the HCV NS5B Thumb-1 pocket showed key residue engagements such as Arg503, which is known to form hydrogen bonds with established HCV Thumb-1 inhibitors. Comparatively, beclabuvir had docking scores of −10.18 kcal/mol and −8.29 kcal/mol, consistent with previous work that predicted beclabuvir's potential to bind and inhibit SARS-CoV-2 RdRp in docking and molecular dynamics studies.

### Example 6

### In Vitro Potency Evaluation

The 15 NCEs (provided in Table 1) selected for experimental validation were successfully synthesized at Piramal Pharma Solutions, with all compounds achieving a purity greater than 95%. This ensured their suitability for downstream validation of bioactivity.

To validate NCE broad-spectrum antiviral activity experimentally, pseudovirus assays specific to HCV 1b and human Coronavirus 229E (CoV229E) (FIG. 7 and Table 6) were used. Sofosbuvir was used as the positive control in the HCV assay, with an EC50 of 0.06 μM, and Remdesivir was used as the positive control in the CoV229E assay, with an EC50 of 0.14 μM. In these assays, all 15 NCEs (provided in Table 1) showed antiviral activity against either HCV or CoV229E, with EC₅₀ values ranging from 0.29-10 μM, though CoV229E was more sensitive than HCV. Specifically, 13 of 15 NCEs inhibited Cov229E. On the other hand, 10 of 15 NCEs inhibited HCV.

TABLE 6
In vitro results of pipendoxifene, fifteen compounds
of Table 1 (“NCE”), and two positive control
compounds against HCV 1b and CoV229E

| | HCV 1b - | HCV 1b - | CoV229E - | CoV229E - |
|---|---|---|---|---|
| Compound ID | EC₅₀ (μM) | TC₅₀ (μM) | EC₅₀ (μM) | TC₅₀ (μM) |
| Pipendoxifene | 3.28 | >10 | 0.11-0.52 | >1 |
| MCAA-01 | 3.61 | >10 | 1.88 | >5 |
| MCAA-02 | >10 | >10 | 2.78 | >5 |
| MCAA-03 | 8.43 | >10 | 24% @ | 2.47 |
| | | | 5 μM | |
| MCAA-04 | >10 | >10 | 25% @ | 2.7 |
| | | | 1.58 μM | |
| MCAA-05 | >10 | >10 | >5 | >5 |
| MCAA-06 | >10 | >10 | 0.29 | 1.53 |
| MCAA-07 | 30% @ | >10 | 11% @ | >5 |
| | 10 μM | | 5 μM | |
| MCAA-08 | 9.59 | >10 | 33% @ | >5 |
| | | | 5 μM | |
| MCAA-09 | >10 | >10 | 13% @ | >5 |
| | | | 5 μM | |
| MCAA-10 | 7.2 | >10 | 2.85 | >5 |
| MCAA-11 | 4.19 | 5.82 | 2.74 | >5 |
| MCAA-12 | 2.78 | >10 | 2.94 | >5 |
| MCAA-13 | 7.05 | >10 | 2.96 | >5 |
| MCAA-14 | 34% @ | >10 | 31% @ | >5 |
| | 10 μM | | 5 μM | |
| MCAA-15 | 38% @ | >10 | >5 | >5 |
| | 10 μM | | | |
| Sofosbuvir | 0.06 | >1 | — | — |
| Remdesivir | — | — | 0.14 | >1 |

Next, the NCEs were tested for selective estrogen regulation modulation activity by using a growth inhibition assay. The assay is performed on MCF7 cells, which are derived from a hormone-dependent breast cancer that stops growing upon estrogen receptor inhibition. Fulvestrant was used as the positive control, demonstrating an IC50 of 0.89 nM. Staurosporine and Camptothecin served as positive controls for cytotoxicity and 1% DMSO as the vehicle control. The results revealed that, in contrast to pipendoxifene, the NCEs lacked MCF7 growth inhibition properties, indicating the successful removal of estrogen receptor binding (FIG. 7 and Table 7).

TABLE 7
In vitro results of pipendoxifene, NCEs, and a positive
control compounds in MCF7 growth inhibition assay

| Compound Name (MCAA) | MCF-7 IC50 (μM) |
|---|---|
| MCAA-01 | >10 |
| MCAA-02 | >10 |
| MCAA-03 | >10 |
| MCAA-04 | 2.24 |
| MCAA-05 | >10 |
| MCAA-06 | 3.77 |
| MCAA-07 | >10 |
| MCAA-08 | >10 |
| MCAA-09 | 1.21 |
| MCAA-10 | 0.53 |
| MCAA-11 | 1.66 |
| MCAA-12 | 1.15 |
| MCAA-13 | 2.47 |
| MCAA-14 | 4.38 |
| MCAA-15 | 0.57 |
| Fulvestrant | 0.00089 |
| Pipendoxifene | 0.00064 |

### Example 7

### Additional Discussion

A study was set out to develop and validate GALILEO as an AI-driven, end-to-end drug discovery pipeline capable of: (1) leveraging a single known representative of a novel chemical class as training data to discover novel compounds within the same class (one-shot learning); (2) generating ultra-large in silico chemical screening libraries; (3) discovering multiparameter-optimized NCEs; and (4) achieving high in vitro hit rates with minimal screening.

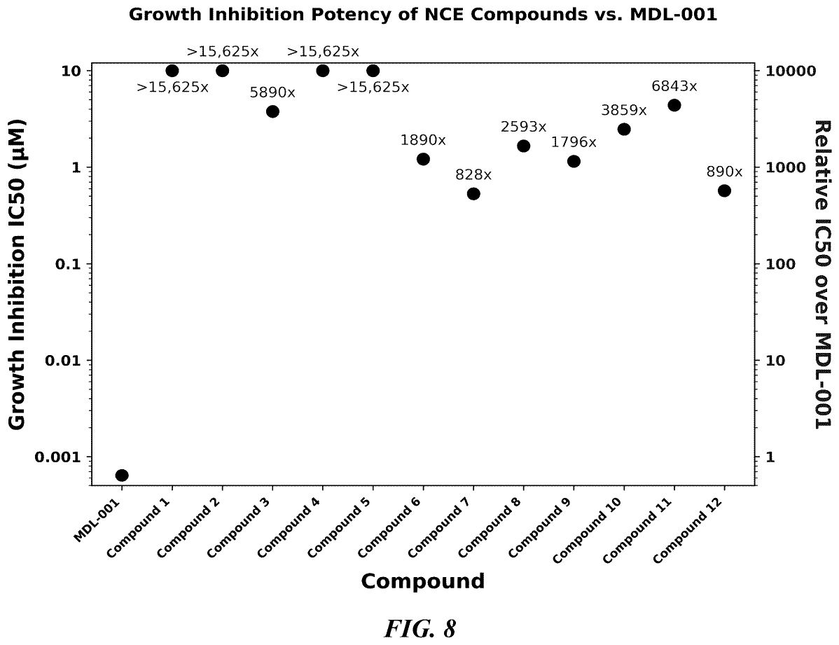

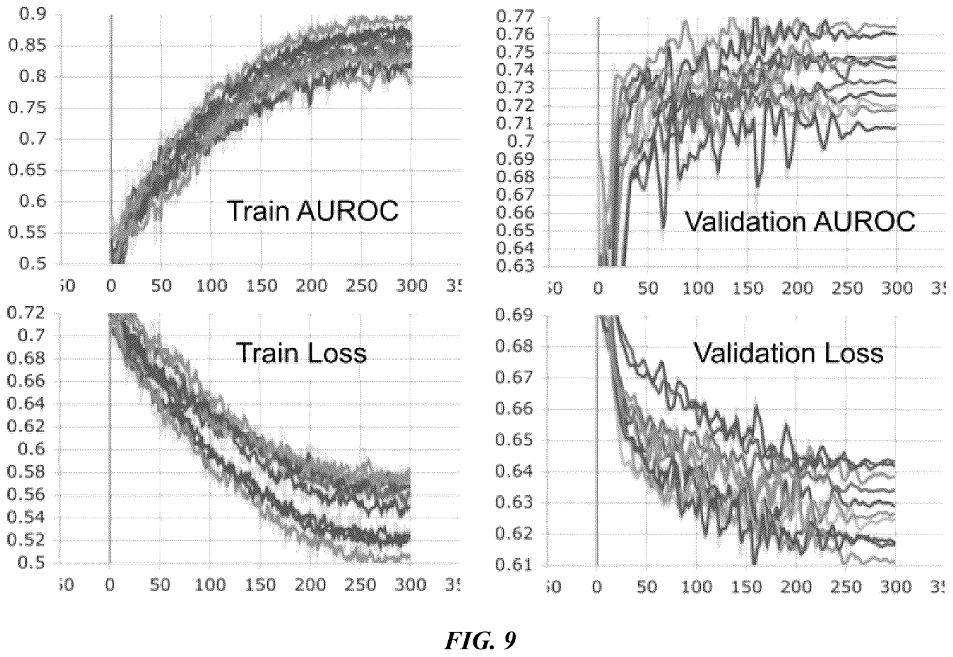

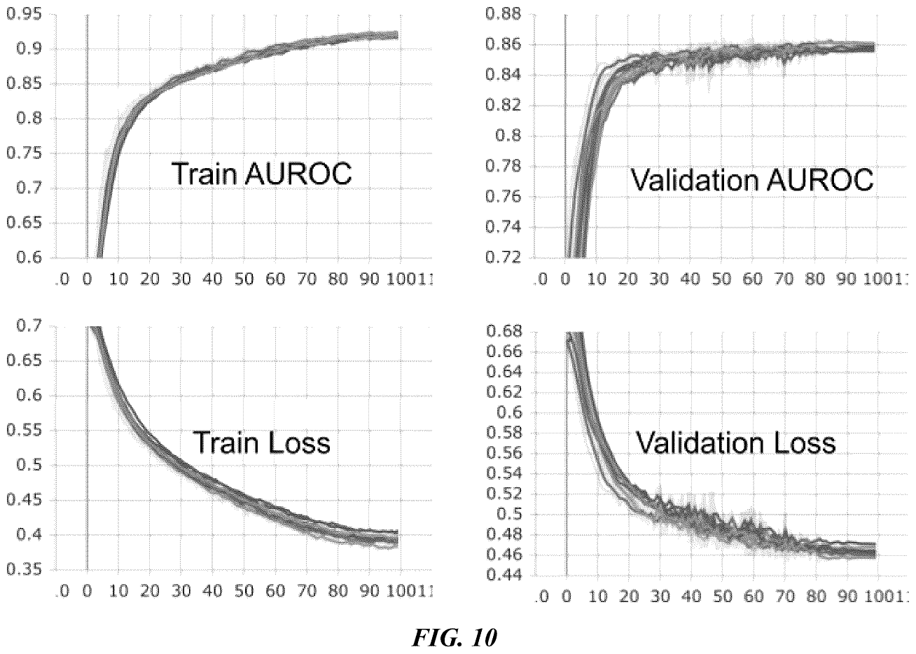

The results demonstrated that GALILEO successfully met these objectives, functioning as a generative, one-shot, multimodal AI drug discovery platform. Specifically, the method used herein: (1) leveraged pipendoxifene, a broad-spectrum antiviral Thumb-1 inhibitor, as training data in a one-shot learning approach to discover 15 novel inhibitors within this same class using ChemPrint (FIGS. 3-4); (2) generated a chemically diverse and relevant screening space of over 50 trillion compounds (FIG. 3); (3) discovered 15 multiparameter-optimized NCEs, based on broad-spectrum antiviral activity, elimination of off-target binding, and chemical diversity (Tables 2-3, FIGS. 3-4 and 6); and (4) achieved a 100% in vitro hit rate (Tables 4-5 and FIGS. 7-8).

Building on the established success of pipendoxifene, a broad-spectrum antiviral therapeutic with demonstrated efficacy and safety, GALILEO developed a next-generation library of broad-spectrum antiviral NCEs with one-shot learning via ChemPrint. Using GALILEO's generative tools, chemical space containing trillions of molecules derived from pipendoxifene's pharmacophoric scaffold was explored. Through a multimodal approach that simultaneously optimized antiviral activity, target specificity, and chemical diversity, ChemPrint's one-shot predictions identified 15 NCEs (provided in Table 1) with predicted broad-spectrum antiviral activity and enhanced target specificity relative to pipendoxifene. Tanimoto similarity analysis demonstrated the chemical novelty of the NCE library, with low similarity to pipendoxifene (average Tanimoto coefficient: 0.38) and Beclabuvir (average Tanimoto coefficient: 0.13), as well as to a curated library of known antiviral drugs. Chemical space mapping further revealed that these NCEs occupy a distinct region, highlighting their uniqueness within the context of existing antiviral therapeutics and ChemPrint's one-shot ability to identify biologically relevant compounds in unexplored areas of chemical space. The 15 identified NCEs provided in Table 1 were synthesized and showed in vitro activity against HCV and/or human Coronavirus 229E, achieving a 100% hit rate. In vitro studies confirmed that the NCEs exhibited specificity improvements of 800-fold to greater than 15,000-fold relative to pipendoxifene's original target. These results highlight GALILEO's capacity to generate optimized, potent, and biologically relevant, drug candidates while eliminating off-target activity with exceptional efficiency.

The 15 NCEs (provided in Table 1) share four attributes: a) high Thumb-1 binding probability; b) broad-spectrum antiviral activity; c) low estrogen receptor binding probability and d) negligible inhibition of estrogen receptor-mediated growth. These NCEs hold great therapeutic value, which must be investigated in detail. Favorable therapeutic properties (oral availability, desirable pharmacokinetics, and clinical safety) are expected for these NCEs, because their chemical structure is based on pipendoxifene's pharmacophoric scaffold. Future studies will assess their pharmacological properties in preclinical settings and their broad-spectrum activity in a large battery of viruses.

This study demonstrates GALILEO as an AI-driven, end-to-end drug discovery platform capable of efficiently generating and optimizing novel therapeutics for novel targets. By creating a trillion-compound NCE space and applying a one-shot, multimodal selection approach, 15 potent antiviral candidates with high specificity and a 100% in vitro hit rate were identified. These results demonstrate that GALILEO accelerates drug discovery by using one-shot learning with ChemPrint to identify multiparameter-optimized therapeutics in previously unexplored chemical space from extraordinarily sparse training data, reducing the time and resources required for drug development.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “of” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for preventing, delaying the onset of, or treating an infection or a disease caused by a ribonucleic acid (RNA) virus, the method comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease, wherein pipendoxifene has a structure of: 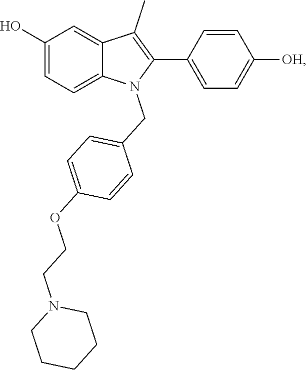 [SMILES: Cc1c(-c2ccc(O)cc2)n(Cc2ccc(OCCN3CCCCC3)cc2)c2ccc(O)cc12]
and wherein the RNA virus is not SARS-CoV-2.

2. The method of claim 1, wherein the RNA virus is a single-stranded RNA virus.

3. The method of claim 1, wherein the RNA virus is a virus in Orthomyxovirae, Flaviviridae, Coronaviridae, or Caliciviridae.

4. The method of claim 1, wherein the RNA virus is an influenza A virus, an influenza B virus, a hepatitis C virus, or a norovirus.

5. The method of claim 1, wherein the RNA virus is not a coronavirus.

6. The method of claim 1, wherein the subject has been or is suspected to have been exposed to, or is at risk of exposure to, the RNA virus.

7. The method of claim 1, wherein the subject is suffering from or at risk of the infection or the disease.

8. The method of claim 1, wherein the infection or the disease is in a lung or liver of the subject.

9. The method of claim 1, wherein the subject is a mammal.

10. The method of claim 1, wherein the composition is in the form of powder, pill, tablet, microtablet, pellet, micropellet, capsule, capsule containing microtablets, liquid, aerosols, or nanoparticles.

11. The method of claim 1, wherein the administering comprises intravenous administration, nasal administration, pulmonary administration, oral administration, parenteral administration, or nebulization.

12. The method of claim 1, wherein administrating the composition reduces or inhibits the viral replication of the RNA virus in the subject as compared to absence of the administering.

13. The method of claim 1, wherein administrating the composition results in a reduction of the viral titer of the RNA virus in the subject as compared to absence of the administering.

14. A method of inhibiting viral activity comprising contacting a cell or tissue with a composition that comprises pipendoxifene having a structure of: 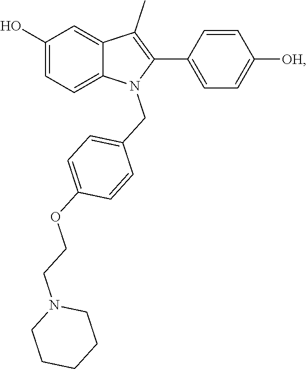 [SMILES: Cc1c(-c2ccc(O)cc2)n(Cc2ccc(OCCN3CCCCC3)cc2)c2ccc(O)cc12]
or a pharmaceutically acceptable salt, ester, solvate, stereoisomer, or tautomer thereof, thereby reducing or inhibiting a viral activity of a ribonucleic acid (RNA) virus encoding an RNA-dependent RNA polymerase (RdRp) in the cell or tissue, wherein the RNA virus is not SARS-CoV-2.

15. The method of claim 14, wherein the RNA virus is a single-stranded RNA virus.

16. The method of claim 14, wherein the RNA virus is a virus in Orthomyxovirae, Flaviviridae, Coronaviridae, or Caliciviridae.

17. The method of claim 14, wherein the RNA virus is an influenza A virus, an influenza B virus, a hepatitis C virus, or a norovirus.

18. The method of claim 14, wherein the RNA virus is not a coronavirus.

19. The method of claim 14, wherein the cell or tissue has been or is suspected to have been exposed to, or is at risk of exposure to, the RNA virus.

20. The method of claim 14, wherein the viral activity is viral replication.

21. The method of claim 14, wherein the contacting occurs in vivo.

22. The method of claim 14, wherein the cell or tissue comprises a lung or liver.
